(19) United States
(12) Patent Application Publication
(10) Pub. No.: US 2024/0368643 A1
(43) Pub. Date: Nov. 7, 2024

(54) METHODS OF PURIFYING CANNABINOIDS

(71) Applicant: Amyris, Inc., Emeryville, CA (US)

(72) Inventors: Benjamin YAP, Emeryville, CA (US); Binita BHATTACHARJEE, Emeryville, CA (US); Dominic VALDES, Emeryville, CA (US); Rudy SITHIRATH, Emeryville, CA (US); Jenna LLOYD-RANDOLFI, Emeryville, CA (US); Tate TONG, Emeryville, CA (US)

(21) Appl. No.: 18/566,902

(22) PCT Filed: Jun. 3, 2022
(86) PCT No.: PCT/US2022/032219

§ 371 Date: Dec. 4, 2023

Related U.S. Application Data
(60) Provisional application No. 63/196,741, filed on Jun. 4, 2021.

(51) Int. Cl. C12P 7/42; C07C 37/68; C07C 51/42; C07D 311/80; C12N 9/54; C12N 15/52; C12P 7/22

(52) U.S. Cl. CPC C12P 7/42; C07C 37/68; C07C 51/42; C07D 311/80; C12N 9/54; C12N 15/52; C12P 7/22; C12Y 304/21062

(57) ABSTRACT

The compositions and methods of the disclosure can be used to purify a cannabinoid in a host cell, such as a yeast cell, genetically modified to express the enzymes of a cannabinoid biosynthetic pathway. Using the compositions and methods of the disclosure, a fermentation composition may be contacted with an enzymatic composition including a serine protease to purify a cannabinoid.

### SEQUENCE LISTING

The instant application contains a Sequence Listing which has been submitted electronically in ASCII format and is hereby incorporated by reference in its entirety. Said ASCII copy, created on Jun. 2, 2022, is named 51494-012WO2_Sequence_Listing_6_2_22_ST25 and is 334,489 bytes in size.

### BACKGROUND OF THE INVENTION

Cannabinoids are chemical compounds such as cannabigerols (CBG), cannabichromens (CBC), cannabidiol (CBD), tetrahydrocannabinol (THC), cannabinol (CBN), cannabinodiol (CBDL), cannabicyclol (CBL), cannabielsoin (CBE), cannabitriol (CBT), and tetrahydrocannabinolic acid (THCa), as well as acid forms thereof, which are produced by the cannabis plant. Cannabinoids may be used to improve various aspects of human health. However, producing cannabinoids in preparative amounts and in high yield has been challenging. There remains a need for methods of purifying cannabinoids with high efficiency and high purity.

### SUMMARY OF THE INVENTION

The present disclosure provides methods for purifying a cannabinoid from a fermentation composition. For example, using the compositions and methods described herein, a cannabinoid may be purified from a fermentation composition produced by culturing host cells genetically modified to express one or more enzymes of a cannabinoid biosynthetic pathway in a culture medium by contacting the fermentation composition with an enzymatic composition that includes a serine protease. The enzymatic composition may be mixed for a time and at a temperature sufficient to allow for demulsification of the fermentation composition before undergoing decarboxylation. Following the decarboxylation, the cannabinoid may be recovered.

In one aspect, the disclosure features a method of purifying a cannabinoid from a fermentation composition including culturing a population of host cells that are genetically modified to express one or more enzymes of a cannabinoid biosynthetic pathway in a culture medium and under conditions suitable for the host cells to produce the cannabinoid, thereby producing a fermentation composition; contacting the fermentation composition with an enzymatic composition including a serine protease; and recovering one or more cannabinoids from the fermentation composition and/or the enzymatic composition.

In another aspect, the disclosure features a method of purifying a cannabinoid from a fermentation composition including providing a fermentation composition that has been produced by culturing a population of host cells that are genetically modified to express one or more enzymes of a cannabinoid biosynthetic pathway in a culture medium and under conditions suitable for the host cells to produce the cannabinoid; contacting the fermentation composition with an enzymatic composition including a serine protease; and recovering one or more cannabinoids from the fermentation composition and/or the enzymatic composition.

In some embodiments, following the culturing of the population of host cells, the fermentation composition is separated into a supernatant and a pellet by solid-liquid centrifugation. In some embodiments, the fermentation composition is contacted with the enzymatic composition after the fermentation is adjusted to a pH of about 7. In some embodiments, the final concentration of the enzymatic composition is from about 0.5% (w/v) to about 1% (w/v) (e.g., 0.6% (w/v), 0.7% (w/v), 0.8% (w/v), 0.9% (w/v), or 1% (w/v)) after contacting the fermentation composition with the enzymatic composition. In some embodiments, the fermentation composition is contacted with the enzymatic composition at a concentration of 1% (w/v) final volume.

In some embodiments, the fermentation composition is mixed with the enzymatic composition for between 0.5 hours and 2 hours (e.g., between 30 minutes and 120 minutes, between 35 minutes and 105 minutes, between 40 minutes and 90 minutes, between 45 minutes and 75 minutes, and between 50 minutes and 60 minutes). In some embodiments, the fermentation composition is mixed with the enzymatic composition for about 60 minutes. In some embodiments, the fermentation composition is maintained at a temperature of 55° C.

In some embodiments, the enzymatic composition includes between 0.003% and 20% serine protease by weight (e.g., between 0.003% and 15%, between 0.005% and 10%, between 0.007% and 7%, and between 0.01% and 5% serine protease by weight). In some embodiments, the enzymatic composition includes between 0.01% and 10% serine protease by weight (e.g., between 0.01% and 10%, between 0.02% and 9%, between 0.03% and 8%, between 0.04% and 7%, between 0.05% and 6%, between 0.06% and 5%, between 0.07% and 4%, between 0.08% and 3%, between 0.08% and 2%, between 0.09% and 1%, and between 0.1% and 1% serine protease by weight). In some embodiments, the enzymatic composition includes between 0.01% and 5% serine protease by weight (e.g., between 0.01% and 5%, between 0.05% and 4%, between 0.1% and 3%, between 0.5% and 2% serine protease by weight).

In some embodiments, the serine protease is a subtilisin. In some embodiments, the subtilisin is from Bacillus licheniformis. In some embodiments, the subtilisin is subtilisin Carlsberg. In some embodiments, the subtilisin has an amino acid sequence that is at least 85% (e.g., at least 85%, 86%, 87%, 88%, 89%, 90%, 91%, 92%, 93%, 94%, 95%, 96%, 97%, 98%, or 99%) identical to the amino acid sequence of SEQ ID NO: 1. In some embodiments, the subtilisin has an amino acid sequence that is at least 90% (e.g., at least 91%, 92%, 93%, 94%, 95%, 96%, 97%, 98%, or 99%) identical to the amino acid sequence of SEQ ID NO: 1. In some embodiments, the subtilisin has an amino acid sequence that is at least 95% (e.g., at least 96%, 97%, 98%, or 99%) identical to the amino acid sequence of SEQ ID NO: 1. In some embodiments, the subtilisin has the amino acid sequence of SEQ ID NO: 1.

In some embodiments, the serine protease is deactivated by exposure to (i) 300 ppm hypochlorite at a temperature of 85° F. for less than one minute; (ii) 3.5 ppm hypochlorite at a temperature of 100° F. for 2 min; or (iii) a pH below 4 for 30 min at a temperature of 140° F. In some embodiments, the serine protease is deactivated by heating to a temperature of 175° F. for 10 min. In some embodiments, the serine protease is deactivated by exposure to liquid/liquid centrifugation at 70° C.

In some embodiments, the enzymatic composition includes an alkylaryl sulfonate salt. In some embodiments, the alkylaryl sulfonate includes a linear alkylaryl sulfonate salt. In some embodiments, the enzymatic composition includes a phosphate salt. In some embodiments, the enzymatic composition includes a carbonate salt. In some embodiments, the salt is a sodium salt.

In some embodiments, the enzymatic composition has a pH of between 8.5 and 11 (e.g., between pH 8.7 and pH 10.5, between pH 9.0 and pH 10, or between pH 9.2 and pH 9.7) in a 1% (w/v) solution. In some embodiments, the enzymatic composition has a pH of about 9.5 in a 1% (w/v) solution.

In some embodiments, the enzymatic composition contains Tergazyme®, a composition that includes a homogeneous blend of sodium linear alkylaryl sulfonate, phosphates, carbonates, and subtilisin Carlsberg.

In some embodiments, the fermentation composition undergoes liquid-liquid centrifugation after being contacted with the enzymatic composition. In some embodiments, the fermentation composition is passed through an evaporator after being contacted with the enzymatic composition. In some embodiments, the fermentation composition is passed through an evaporator more than once (e.g., twice). In some embodiments, the walls of the evaporator are heated to a temperature of about 180° C. In some embodiments, the walls of the evaporator are heated to a temperature of about 250° C. In some embodiments, the condenser of the evaporator is heated to a temperature of 80° C. In some embodiments, the walls of the evaporator are heated to a temperature of about 180° C. and the condenser of the evaporator is heated to a temperature of 80° C. the first time the fermentation composition is passed through the evaporator, and the walls of the evaporator are heated to a temperature of about 250° C. and the condenser of the evaporator is heated to a temperature of 80° C. the second time the fermentation composition is passed through the evaporator. In some embodiments, the evaporate is a short-path evaporator (e.g., a wiped-film evaporator). In some embodiments, the fermentation composition is heated to a temperature of 180° C. or more for less than 5 minutes (e.g., 1 minute, 2 minutes, 3 minutes, and 4 minutes). In some embodiments, the fermentation composition is heated to a temperature of 180° C. or more for less than 1 minute (e.g., less than 55 seconds, 50 seconds, 45 seconds, 40 seconds, 35 seconds, 30 seconds, 25 seconds, 20 seconds, 15 seconds, 10 seconds, and 5 seconds).

In some embodiments, the cannabinoid is recovered using crystallization after the fermentation solution is passed through the evaporator.

In some embodiments, the recovered cannabinoid has between 50% and 100% purity (e.g., between 55% and 95%, between 60% and 90%, between 65% and 85%, and between 70% and 80% purity). In some embodiments, the recovered cannabinoid has between 70% and 100% purity (e.g., between 75%, and 95%, and between 80% and 90% purity). In some embodiments, the molar yield of the cannabinoid is between 60% and 100% (e.g., between 65% and 95%, between 70% and 90%, and between 75% and 85%). In some embodiments, the molar yield is between 90% and 100% (e.g., 91%, 92%, 93%, 94%, 95%, 96%, 97%, 98%, or 99%).

In some embodiments, the host cells include one or more heterologous nucleic acids that each, independently, encode an acyl activating enzyme (AAE), and/or a tetraketide synthase (TKS), and/or a cannabigerolic acid synthase (CBGaS), and/or a geranyl pyrophosphate (GPP) synthase. In some embodiments, the host cells include heterologous nucleic acids that independently encode an AAE, a TKS, a CBGaS, and a GPP synthase.

In some embodiments, the host cell includes a heterologous nucleic acid that encodes an AAE having an amino acid sequence that is at least 90% (e.g., at least 91%, 92%, 93%, 94%, 95%, 96%, 97%, 98%, or 99%) identical to the amino acid sequence of any one of SEQ ID NO: 2-25. In some embodiments, the AAE has an amino acid sequence that is at least 95% (e.g., at least 96%, 97%, 98%, or 99%) identical to the amino acid sequence of any one of SEQ ID NO: 2-25. In some embodiments, the AAE has the amino acid sequence of any one of SEQ ID NO: 2-25.

In some embodiments, the AAE has an amino acid sequence that is at least 90% (e.g., at least 91%, 92%, 93%, 94%, 95%, 96%, 97%, 98%, or 99%) identical to the amino acid sequence of any one of SEQ ID NO: 2-14. In some embodiments, the AAE has an amino acid sequence that is at least 95% (e.g., at least 96%, 97%, 98%, or 99%) identical to the amino acid sequence of any one of SEQ ID NO: 2-14. In some embodiments, the AAE has the amino acid sequence of any one of SEQ ID NO: 2-14.

In some embodiments, the AAE has an amino acid sequence that is at least 90% (e.g., at least 91%, 92%, 93%, 94%, 95%, 96%, 97%, 98%, or 99%) identical to the amino acid sequence of any one of SEQ ID NO: 2-6. In some embodiments, the AAE has an amino acid sequence that is at least 95% (e.g., at least 96%, 97%, 98%, or 99%) identical to the amino acid sequence of any one of SEQ ID NO: 2-6. In some embodiments, the AAE has the amino acid sequence of any one of SEQ ID NO: 2-6.

In some embodiments, the host cell includes a heterologous nucleic acid that encodes a TKS having an amino acid sequence that is at least 90% (e.g., at least 91%, 92%, 93%, 94%, 95%, 96%, 97%, 98%, or 99%) identical to the amino acid sequence of any one SEQ ID NO: 26-60. In some embodiments, the TKS has an amino acid sequence that is at least 95% (e.g., at least 96%, 97%, 98%, or 99%) identical to the amino acid sequence of any one of SEQ ID NO: 26-60. In some embodiments, the TKS has the amino acid sequence of any one of SEQ ID NO: 26-60.

In some embodiments, the TKS has an amino acid sequence that is at least 90% (e.g., at least 91%, 92%, 93%, 94%, 95%, 96%, 97%, 98%, or 99%) identical to the amino acid sequence of any one SEQ ID NO: 26-29. In some embodiments, the TKS has an amino acid sequence that is at least 95% (e.g., at least 96%, 97%, 98%, or 99%) identical to the amino acid sequence of any one of SEQ ID NO: 26-29. In some embodiments, the TKS has the amino acid sequence of any one of SEQ ID NO: 26-29.

In some embodiments, the TKS has an amino acid sequence that is at least 90% (e.g., at least 91%, 92%, 93%, 94%, 95%, 96%, 97%, 98%, or 99%) identical to the amino acid sequence of SEQ ID NO: 26. In some embodiments, the TKS has an amino acid sequence that is at least 95% (e.g., at least 96%, 97%, 98%, or 99%) identical to the amino acid sequence of SEQ ID NO: 26. In some embodiments, the TKS has the amino acid sequence of SEQ ID NO: 26.

In some embodiments, the host cell includes a heterologous nucleic acid that encodes a CBGaS having an amino acid sequence that is at least 90% (e.g., at least 91%, 92%, 93%, 94%, 95%, 96%, 97%, 98%, or 99%) identical to the amino acid sequence of any one of SEQ ID NO: 61-65. In some embodiments, the CBGaS has an amino acid sequence that is at least 95% (e.g., at least 96%, 97%, 98%, or 99%) identical to the amino acid sequence of any one of SEQ ID NO: 61-65. In some embodiments, the CBGaS has the amino acid sequence of any one of SEQ ID NO: 61-65.

In some embodiments, the host cell includes a heterologous nucleic acid that encodes a GPP synthase having an amino acid sequence that is at least 90% (e.g., at least 91%, 92%, 93%, 94%, 95%, 96%, 97%, 98%, or 99%) identical to the amino acid sequence of any one of SEQ ID NO: 66-71. In some embodiments, the GPP synthase has an amino acid sequence that is at least 95% (e.g., at least 96%, 97%, 98%, or 99%) identical to the amino acid sequence of any one of SEQ ID NO: 66-71. In some embodiments, the GPP synthase has the amino acid sequence of any one of SEQ ID NO: 66-71.

In some embodiments, the GPP synthase has an amino acid sequence that is at least 90% (e.g., at least 91%, 92%, 93%, 94%, 95%, 96%, 97%, 98%, or 99%) identical to the amino acid sequence of SEQ ID NO: 66. In some embodiments, the GPP synthase has an amino acid sequence that is at least 95% (e.g., at least 96%, 97%, 98%, or 99%) identical to the amino acid sequence of SEQ ID NO: 66. In some embodiments, the GPP synthase has the amino acid sequence of SEQ ID NO: 66.

In some embodiments, the host cell includes heterologous nucleic acids that independently encode an AAE having the amino acid sequence of any one of SEQ ID NO: 2-25, a TKS having the amino acid sequence of any one of SEQ ID NO: 26-60, a CBGaS having the amino acid sequences of any one of SEQ ID NO: 61-65, and a GPP synthase having the amino acid sequence of any one of SEQ ID NO: 66-71.

In some embodiments, the host cell further includes one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase. In some embodiments, the host cell includes heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

In some embodiments, the host cell further includes a heterologous nucleic acid that encodes an olivetolic acid cyclase (OAC). In some embodiments, the OAC has an amino acid sequence that is at least 90% (e.g., at least 91%, 92%, 93%, 94%, 95%, 96%, 97%, 98%, or 99%) identical to the amino acid sequence of SEQ ID NO: 72. In some embodiments, the OAC has an amino acid sequence that is at least 95% (e.g., at least 96%, 97%, 98%, or 99%) identical to the amino acid sequence of SEQ ID NO: 72. In some embodiments, the OAC has the amino acid sequence of SEQ ID NO: 72.

In some embodiments, the host cell further includes one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 73. In some embodiments, the acetyl-CoA synthase has an amino acid sequence that is at least 95% (e.g., at least 96%, 97%, 98%, or 99%) identical to the amino acid sequence of SEQ ID NO: 73. In some embodiments, the acetyl-CoA synthase has the amino acid sequence of SEQ ID NO: 73.

In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 74. In some embodiments, the acetyl-CoA synthase has an amino acid sequence that is at least 95% (e.g., at least 96%, 97%, 98%, or 99%) identical to the amino acid sequence of SEQ ID NO: 74. In some embodiments, the acetyl-CoA synthase has the amino acid sequence of SEQ ID NO: 74.

In some embodiments, the aldehyde dehydrogenase has an amino acid sequence that is at least 90% (e.g., at least 91%, 92%, 93%, 94%, 95%, 96%, 97%, 98%, or 99%) identical to the amino acid sequence of SEQ ID NO: 75. In some embodiments, the aldehyde dehydrogenase has an amino acid sequence that is at least 95% (e.g., at least 96%, 97%, 98%, or 99%) identical to the amino acid sequence of SEQ ID NO: 75. In some embodiments, the aldehyde dehydrogenase synthase has the amino acid sequence of SEQ ID NO: 75.

In some embodiments, the pyruvate decarboxylase has an amino acid sequence that is at least 90% (e.g., at least 91%, 92%, 93%, 94%, 95%, 96%, 97%, 98%, or 99%) identical to the amino acid sequence of SEQ ID NO: 76. In some embodiments, the pyruvate decarboxylase has an amino acid sequence that is at least 95% (e.g., at least 96%, 97%, 98%, or 99%) identical to the amino acid sequence of SEQ ID NO: 76. In some embodiments, the pyruvate decarboxylase has the amino acid sequence of SEQ ID NO: 76.

In some embodiments, the host cell contains a heterologous nucleic acid encoding an aceto-CoA carboxylase (ACC). In some embodiments, the heterologous nucleic acid encodes a ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78.

In some embodiments, the host cell contains a heterologous nucleic acid encoding an ACC and an acetoacetyl-CoA synthase (AACS) instead of a heterologous nucleic acid encoding an acetyl-CoA thiolase. In some embodiments, the heterologous nucleic acid encodes an ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78. In some embodiments, the heterologous nucleic acid encodes an AACS having an amino acid sequence that is at least 90% identical to the amino acid sequence of SEQ ID NO: 77 (e.g., an amino acid sequence that is 90%, 91%, 92%, 93%, 94%, 95%, 96%, 97%, 98%, 99%, or 100% identical to SEQ ID NO: 77). In some embodiments, the AACS has an amino acid sequence that is at least 95% identical to the amino acid sequence of SEQ ID NO: 77 (e.g., an amino acid sequence that is 95%, 96%, 97%, 98%, 99%, or 100% identical to SEQ ID NO: 77). In some embodiments, the AACS has the amino acid sequence of SEQ ID NO: 77.

In some embodiments, expression of the one or more heterologous nucleic acids are regulated by an exogenous agent. In some embodiments, the exogenous agent includes a regulator of gene expression. In some embodiments, the exogenous agent decreases production of the cannabinoid. In some embodiments, the exogenous agent is maltose. In some embodiments, the exogenous agent increases production of the cannabinoid. In some embodiments, the exogenous agent is galactose. In some embodiments, the exogenous agent is galactose and expression of one or more heterologous nucleic acids encoding the AAE, TKS, and CBGaS enzymes is under the control of a GAL promoter. In some embodiments, expression of one or more heterologous nucleic acids encoding the AAE, TKS, and CBGaS enzymes is under the control of a galactose-responsive promoter, a maltose-responsive promoter, or a combination of both.

In some embodiments, the method includes culturing the host cell with the precursor required to make the cannabinoid. In some embodiments, the precursor required to make the cannabinoid is hexanoate. In some embodiments, the cannabinoid is cannabidiolic acid (CBDA), cannabidiol (CBD), cannabigerolic acid (CBGA), cannabigerol (CBG), tetrahydrocannabinol (THC), or tetrahydrocannabinolic acid (THCa). In some embodiments, the host cell is a yeast cell or yeast strain. In some embodiments, the yeast cell is S. cerevisiae.

In another aspect, the disclosure provides a method of decarboxylating a cannabinoid including contacting an enzymatic composition including a serine protease with a fermentation composition, wherein the fermentation composition includes a population of host cells that are genetically modified to express one or more enzymes of a cannabinoid biosynthetic pathway; and has been cultured in a culture medium and under conditions suitable for the host cells to produce the cannabinoid.

In some embodiments, the fermentation composition is separated into a supernatant and a pellet by solid-liquid centrifugation. In some embodiments, following the culturing of the population of host cells, the fermentation composition is separated into a supernatant and a pellet by solid-liquid centrifugation. In some embodiments, the fermentation composition is contacted with the enzymatic composition after the fermentation is adjusted to a pH of about 7. In some embodiments, the final concentration of the enzymatic composition is from about 0.5% (w/v) to about 3% (w/v) (e.g., 0.6% (w/v), 0.7% (w/v), 0.8% (w/v), 0.9% (w/v), 1% (w/v), 1.5% (w/V), 2% (w/v), 2.5% (w/v) and 3% (w/v)) after contacting the fermentation composition with the enzymatic composition. In some embodiments, the fermentation composition is contacted with the enzymatic composition at a concentration of 1% (w/v) final volume.

In some embodiments, the fermentation composition is mixed with the enzymatic composition for between 0.5 hours and 2 hours (e.g., between 30 minutes and 120 minutes, between 35 minutes and 105 minutes, between 40 minutes and 90 minutes, between 45 minutes and 75 minutes, and between 50 minutes and 60 minutes). In some embodiments, the fermentation composition is mixed with the enzymatic composition for about 60 minutes. In some embodiments, the fermentation composition is maintained at a temperature of 55° C.

In some embodiments, the enzymatic composition includes between 0.003% and 20% serine protease by weight (e.g., between 0.003% and 15%, between 0.005% and 10%, between 0.007% and 7%, and between 0.01% and 5% serine protease by weight). In some embodiments, the enzymatic composition includes between 0.01% and 10% serine protease by weight (e.g., between 0.01% and 10%, between 0.02% and 9%, between 0.03% and 8%, between 0.04% and 7%, between 0.05% and 6%, between 0.06% and 5%, between 0.07% and 4%, between 0.08% and 3%, between 0.08% and 2%, between 0.09% and 1%, and between 0.1% and 1% serine protease by weight). In some embodiments, the enzymatic composition includes between 0.01% and 5% by serine protease by weight (e.g., between 0.01% and 5%, between 0.05% and 4%, between 0.1% and 3%, between 0.5% and 2% serine protease by weight).

In some embodiments, the serine protease is a subtilisin. In some embodiments, the subtilisin is from Bacillus licheniformis. In some embodiments, the subtilisin is subtilisin Carlsberg. In some embodiments, the subtilisin has an amino acid sequence that is at least 85% (e.g., at least 85%, 86%, 87%, 88%, 89%, 90%, 91%, 92%, 93%, 94%, 95%, 96%, 97%, 98%, or 99%) identical to the amino acid sequence of SEQ ID NO: 1. In some embodiments, the subtilisin has an amino acid sequence that is at least 90% (e.g., at least 91%, 92%, 93%, 94%, 95%, 96%, 97%, 98%, or 99%) identical to the amino acid sequence of SEQ ID NO: 1. In some embodiments, the subtilisin has an amino acid sequence that is at least 95% e.g., at least 95%, 96%, 97%, 98%, or 99%) identical to the amino acid sequence of SEQ ID NO: 1. In some embodiments, the subtilisin has the amino acid sequence of SEQ ID NO: 1.

In some embodiments, the enzymatic composition includes an alkylaryl sulfonate salt. In some embodiments, the alkylaryl sulfonate includes a linear alkylaryl sulfonate salt. In some embodiments, the enzymatic composition includes a phosphate salt. In some embodiments, the enzymatic composition includes a carbonate salt. In some embodiments, the salt is a sodium salt.

In some embodiments, the enzymatic composition has a pH of between 8.5 and 11 (e.g., between pH 8.7 and pH 10.5, between pH 9.0 and pH 10, and between pH 9.2 and pH 9.7) in a 1% (w/v) solution. In some embodiments, the enzymatic composition has a pH of about 9.5 in a 1% (w/v) solution. In some embodiments, the fermentation composition undergoes liquid-liquid centrifugation after being contacted with the enzymatic composition. In some embodiments, the fermentation composition is passed through an evaporator after being contacted with the enzymatic composition. In some embodiments, the fermentation composition is passed through the evaporator more than once. In some embodiments, the fermentation composition is passed through the evaporator twice. In some embodiments, the walls of the evaporator are heated to a temperature of about 180° C. In some embodiments, the walls of the evaporator are heated to a temperature of about 250° C. In some embodiments, the condenser of the evaporator is heated to a temperature of 80° C. In some embodiments, the walls of the evaporator are heated to a temperature of about 180° C. and the condenser of the evaporator is heated to a temperature of 80° C. the first time the fermentation composition is passed through the evaporator, and the walls of the evaporator are heated to a temperature of about 250° C. and the condenser of the evaporator is heated to a temperature of 80° C. the second time the fermentation composition is passed through the evaporator. In some embodiments, the evaporate is a short-path evaporator (e.g., a wiped-film evaporator). In some embodiments, the fermentation composition is heated to a temperature of 180° C. or more for less than 5 minutes (e.g., 4 minutes, 3 minutes, 2 minutes, and 1 minute). In some embodiments, the fermentation composition is heated to a temperature of 180° C. or more for less than 1 minute (e.g., less than 55 seconds, 50 seconds, 45 seconds, 40 seconds, 35 seconds, 30 seconds, 25 seconds, 20 seconds, 15 seconds, 10 seconds, and 5 seconds).

In some embodiments, the host cells include one or more heterologous nucleic acids that each, independently, encode an AAE, and/or a TKS, and/or a CBGaS, and/or a GPP synthase. In some embodiments, the host cells include heterologous nucleic acids that independently encode an AAE, a TKS, a CBGaS, and a GPP synthase.

In some embodiments, the AAE has an amino acid sequence that is at least 90% (e.g., at least 91%, 92%, 93%, 94%, 95%, 96%, 97%, 98%, or 99%) identical to the amino acid sequence of any one of SEQ ID NO: 2-25. In some embodiments, the AAE has an amino acid sequence that is at least 95% (e.g., at least 96%, 97%, 98%, or 99%) identical to the amino acid sequence of any one of SEQ ID NO: 2-25. In some embodiments, the AAE has the amino acid sequence of any one of SEQ ID NO: 2-25. In some embodiments, the AAE has an amino acid sequence that is at least 90% (e.g., at least 91%, 92%, 93%, 94%, 95%, 96%, 97%, 98%, or 99%) identical to the amino acid sequence of any one of SEQ ID NO: 2-14. In some embodiments, the AAE has an amino acid sequence that is at least 95% (e.g., at least 96%, 97%, 98%, or 99%) identical to the amino acid sequence of any one of SEQ ID NO: 2-14. In some embodiments, the AAE has the amino acid sequence of any one of SEQ ID NO: 2-14. In some embodiments, the AAE has an amino acid sequence that is at least 90% (e.g., at least 91%, 92%, 93%, 94%, 95%, 96%, 97%, 98%, or 99%) identical to the amino acid sequence of any one of SEQ ID NO: 2-6. In some embodiments, the AAE has an amino acid sequence that is at least 95% (e.g., at least 96%, 97%, 98%, or 99%) identical to the amino acid sequence of any one of SEQ ID NO: 2-6. In some embodiments, the AAE has the amino acid sequence of any one of SEQ ID NO: 2-6.

In some embodiments, the TKS has an amino acid sequence that is at least 90% (e.g., at least 91%, 92%, 93%, 94%, 95%, 96%, 97%, 98%, or 99%) identical to the amino acid sequence of any one of SEQ ID NO: 26-60. In some embodiments, the TKS has an amino acid sequence that is at least 95% (e.g., at least 96%, 97%, 98%, or 99%) identical to the amino acid sequence of any one of SEQ ID NO: 26-60. In some embodiments, the TKS has the amino acid sequence of any one of SEQ ID NO: 26-60. In some embodiments, the TKS has an amino acid sequence that is at least 90% (e.g., at least 91%, 92%, 93%, 94%, 95%, 96%, 97%, 98%, or 99%) identical to the amino acid sequence of any one of SEQ ID NO: 26-29. In some embodiments, the TKS has an amino acid sequence that is at least 95% (e.g., at least 96%, 97%, 98%, or 99%) identical to the amino acid sequence of any one of SEQ ID NO: 26-29. In some embodiments, the TKS has the amino acid sequence of any one of SEQ ID NO: 26-29. In some embodiments, the TKS has an amino acid sequence that is at least 90% (e.g., at least 91%, 92%, 93%, 94%, 95%, 96%, 97%, 98%, or 99%) identical to the amino acid sequence of SEQ ID NO: 26. In some embodiments, the TKS has an amino acid sequence that is at least 95% (e.g., at least 96%, 97%, 98%, or 99%) identical to the amino acid sequence of SEQ ID NO: 26. In some embodiments, the TKS has the amino acid sequence of SEQ ID NO: 26.

In some embodiments, the CBGaS has an amino acid sequence that is at least 90% (e.g., at least 91%, 92%, 93%, 94%, 95%, 96%, 97%, 98%, or 99%) identical to the amino acid sequence of any one of SEQ ID NO: 61-65. In some embodiments, the CBGaS has an amino acid sequence that is at least 95% (e.g., at least 96%, 97%, 98%, or 99%) identical to the amino acid sequence of any one of SEQ ID NO: 61-65. In some embodiments, the CBGaS has the amino acid sequence of any one of SEQ ID NO: 61-65.

In some embodiments, the GPP synthase has an amino acid sequence that is at least 90% (e.g., at least 91%, 92%, 93%, 94%, 95%, 96%, 97%, 98%, or 99%) identical to the amino acid sequence of any one of SEQ ID NO: 66-71. In some embodiments, the GPP synthase has an amino acid sequence that is at least 95% (e.g., at least 96%, 97%, 98%, or 99%) identical to the amino acid sequence of any one of SEQ ID NO: 66-71. In some embodiments, the GPP synthase has the amino acid sequence of any one of SEQ ID NO: 66-71. In some embodiments, the GPP synthase has an amino acid sequence that is at least 90% (e.g., at least 91%, 92%, 93%, 94%, 95%, 96%, 97%, 98%, or 99%) identical to the amino acid sequence of SEQ ID NO: 66. In some embodiments, the GPP synthase has an amino acid sequence that is at least 95% (e.g., at least 96%, 97%, 98%, or 99%) identical to the amino acid sequence of SEQ ID NO: 66. In some embodiments, the GPP synthase has the amino acid sequence of SEQ ID NO: 66.

In some embodiments, the host cell includes heterologous nucleic acids that independently encode an AAE having the amino acid sequence of any one of SEQ ID NO: 2-25, a TKS having the amino acid sequence of any one of SEQ ID NO: 26-60, a CBGaS having the amino acid sequences of any one of SEQ ID NO: 61-65, and a GPP synthase having the amino acid sequence of any one of SEQ ID NO: 66-71.

In some embodiments, the host cell further includes one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase. In some embodiments, the host cell includes heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

In some embodiments, the host cell further includes a heterologous nucleic acid that encodes an OAC. In some embodiments, the OAC has an amino acid sequence that is at least 90% (e.g., at least 91%, 92%, 93%, 94%, 95%, 96%, 97%, 98%, or 99%) identical to the amino acid sequence of SEQ ID NO: 72. In some embodiments, the OAC has an amino acid sequence that is at least 95% (e.g., at least 96%, 97%, 98%, or 99%) identical to the amino acid sequence of SEQ ID NO: 72. In some embodiments, the OAC has the amino acid sequence of SEQ ID NO: 72.

In some embodiments, the host cell further includes one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 73. In some embodiments, the acetyl-CoA synthase has an amino acid sequence that is at least 95% (e.g., at least 96%, 97%, 98%, or 99%) identical to the amino acid sequence of SEQ ID NO: 73. In some embodiments, the acetyl-CoA synthase has the amino acid sequence of SEQ ID NO: 73.

In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 74. In some embodiments, the acetyl-CoA synthase has an amino acid sequence that is at least 95% (e.g., at least 96%, 97%, 98%, or 99%) identical to the amino acid sequence of SEQ ID NO: 74. In some embodiments, the acetyl-CoA synthase has the amino acid sequence of SEQ ID NO: 74.

In some embodiments, the aldehyde dehydrogenase has an amino acid sequence that is at least 90% (e.g., at least 91%, 92%, 93%, 94%, 95%, 96%, 97%, 98%, or 99%) identical to the amino acid sequence of SEQ ID NO: 75. In some embodiments, the aldehyde dehydrogenase has an amino acid sequence that is at least 95% (e.g., at least 96%, 97%, 98%, or 99%) identical to the amino acid sequence of SEQ ID NO: 75. In some embodiments, the aldehyde dehydrogenase synthase has the amino acid sequence of SEQ ID NO: 75.

In some embodiments, the pyruvate decarboxylase has an amino acid sequence that is at least 90% (e.g., at least 91%, 92%, 93%, 94%, 95%, 96%, 97%, 98%, or 99%) identical to the amino acid sequence of SEQ ID NO: 76. In some embodiments, the pyruvate decarboxylase has an amino acid sequence that is at least 95% (e.g., at least 96%, 97%, 98%, or 99%) identical to the amino acid sequence of SEQ ID NO: 76. In some embodiments, the pyruvate decarboxylase has the amino acid sequence of SEQ ID NO: 76.

In some embodiments, the host cell contains a heterologous nucleic acid encoding an ACC. In some embodiments, the heterologous nucleic acid encodes a ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78.

In some embodiments, the host cell contains a heterologous nucleic acid encoding an ACC and an AACS instead of a heterologous nucleic acid encoding an acetyl-CoA thiolase. In some embodiments, the heterologous nucleic acid encodes an ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78. In some embodiments, the heterologous nucleic acid encodes an AACS having an amino acid sequence that is at least 90% identical to the amino acid sequence of SEQ ID NO: 77 (e.g., an amino acid sequence that is 90%, 91%, 92%, 93%, 94%, 95%, 96%, 97%, 98%, 99%, or 100% identical to SEQ ID NO: 77). In some embodiments, the AACS has an amino acid sequence that is at least 95% identical to the amino acid sequence of SEQ ID NO: 77 (e.g., an amino acid sequence that is 95%, 96%, 97%, 98%, 99%, or 100% identical to SEQ ID NO: 77). In some embodiments, the AACS has the amino acid sequence of SEQ ID NO: 77.

In some embodiments, expression of the one or more heterologous nucleic acids are regulated by an exogenous agent. In some embodiments, the exogenous agent includes a regulator of gene expression. In some embodiments, the exogenous agent decreases production of the cannabinoid. In some embodiments, the exogenous agent is maltose. In some embodiments, the exogenous agent increases production of the cannabinoid. In some embodiments, the exogenous agent is galactose. In some embodiments, the exogenous agent is galactose and expression of one or more heterologous nucleic acids encoding the AAE, TKS, and CBGaS enzymes is under the control of a GAL promoter. In some embodiments, expression of one or more heterologous nucleic acids encoding the AAE, TKS, and CBGaS enzymes is under the control of a galactose-responsive promoter, a maltose-responsive promoter, or a combination of both.

In some embodiments, the method includes culturing the host cell with the precursor required to make the cannabinoid. In some embodiments, the precursor required to make the cannabinoid is hexanoate. In some embodiments, the cannabinoid is CBDA, CBD, CBGA, CBG, THC, THCa. In some embodiments, the host cell is a yeast cell or yeast strain. In some embodiments, the yeast cell is S. cerevisiae.

In another aspect, the disclosure provides a mixture including a fermentation composition produced by culturing a population of host cells that are genetically modified to express one or more enzymes of a cannabinoid biosynthetic pathway in a culture medium and under conditions suitable for the host cells to produce the cannabinoid; and an enzymatic composition including a serine protease. In some embodiments, the serine protease is a wherein the serine protease is a subtilisin from Bacillus licheniformis. In some embodiments, the enzymatic composition includes sodium linear alkylaryl sulfonates, phosphates, and carbonates. In some embodiments, the host cells include one or more heterologous nucleic acids that each, independently, encode an AAE, and/or TKS, and/or CBGaS, and/or GPP synthase. In some embodiments, the host cell further includes one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

### Definitions

As used herein the singular forms “a,” “an,” and, “the” include plural reference unless the context clearly dictates otherwise.

The term “about” when modifying a numerical value or range herein includes normal variation encountered in the field, and includes plus or minus 1-10% (e.g., 1%, 2%, 3%, 4%, 5%, 6%, 7%, 8%, 9% or 10%) of the numerical value or end points of the numerical range. Thus, a value of 10 includes all numerical values from 9 to 11. All numerical ranges described herein include the endpoints of the range unless otherwise noted, and all numerical values in-between the end points, to the first significant digit.

As used herein, the term “cannabinoid” refers to a chemical substance that binds or interacts with a cannabinoid receptor (for example, a human cannabinoid receptor) and includes, without limitation, chemical compounds such endocannabinoids, phytocannabinoids, and synthetic cannabinoids. Synthetic compounds are chemicals made to mimic phytocannabinoids which are naturally found in the cannabis plant (e.g., Cannabis sativa), including but not limited to cannabigerols (CBG), cannabichromenes (CBC), cannabidiol (CBD), tetrahydrocannabinol (THC), cannabinol (CBN), cannabinodiol (CBDL), cannabicyclol (CBL), cannabielsoin (CBE), and cannabitriol (CBT).

As used herein, the term “capable of producing” refers to a host cell which is genetically modified to include the enzymes necessary for the production of a given compound in accordance with a biochemical pathway that produces the compound. For example, a cell (e.g., a yeast cell) that is “capable of producing” a cannabinoid is one that contains the enzymes necessary for production of the cannabinoid according to the cannabinoid biosynthetic pathway.

As used herein, the term “conservatively modified variants” refers to nucleic acid or amino acid sequences that are substantially identical to a reference. With respect to particular nucleic acid sequences, conservatively modified variants refer to those nucleic acids which encode identical or essentially identical amino acid sequences, or where the nucleic acid does not encode an amino acid sequence to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, in a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Examples of amino acid groups defined in this manner can include: a “charged/polar group” including Glu (Glutamic acid or E), Asp (Aspartic acid or D), Asn (Asparagine or N), Gln (Glutamine or Q), Lys (Lysine or K), Arg (Arginine or R) and His (Histidine or H); an “aromatic or cyclic group” including Pro (Proline or P), Phe (Phenylalanine or F), Tyr (Tyrosine or Y) and Trp (Tryptophan or W); and an “aliphatic group” including Gly (Glycine or G), Ala (Alanine or A), Val (Valine or V), Leu (Leucine or L), Ile (Isoleucine or I), Met (Methionine or M), Ser (Serine or S), Thr (Threonine or T) and Cys (Cysteine or C). Within each group, subgroups can also be identified. For example, at pH 7, the group of charged/polar amino acids can be sub-divided into sub-groups including: the “positively-charged sub-group” comprising Lys, Arg and His; the “negatively-charged sub-group” comprising Glu and Asp; and the “polar sub-group” comprising Asn and Gln. In another example, the aromatic or cyclic group can be sub-divided into sub-groups including: the “nitrogen ring sub-group” comprising Pro, His and Trp; and the “phenyl sub-group” comprising Phe and Tyr. In another further example, the aliphatic group can be sub-divided into sub-groups including: the “large aliphatic non-polar sub-group” comprising Val, Leu and Ile; the “aliphatic slightly-polar sub-group” comprising Met, Ser, Thr and Cys; and the “small-residue sub-group” comprising Gly and Ala. Examples of conservative mutations include amino acid substitutions of amino acids within the sub-groups above, such as, but not limited to: Lys for Arg or vice versa, such that a positive charge can be maintained; Glu for Asp or vice versa, such that a negative charge can be maintained; Ser for Thr or vice versa, such that a free —OH can be maintained; and Gln for Asn or vice versa, such that a free —NH₂ can be maintained. The following six groups each contain amino acids that further provide illustrative conservative substitutions for one another: 1) Ala, Ser, Thr; 2) Asp, Glu; 3) Asn, Gln; 4) Arg, Lys; 5) Ile, Leu, Met, Val; and 6) Phe, Try, and Trp (see, e.g., Creighton, Proteins (1984)).

As used herein, the term “endogenous” refers to a substance or process that can occur naturally in a host cell. In contrast, the term “exogenous” refers a substance or compound that originated outside an organism or cell. The exogenous substance or compound can retain its normal function or activity when introduced into an organism or host cell described herein.

As used herein, the term “enzymatic composition” refers to a composition including at least one enzyme (e.g., a serine protease).

As used herein, the term “expression cassette” or “expression construct” refers to a nucleic acid construct that, when introduced into a host cell, results in transcription and/or translation of an RNA or polypeptide, respectively. In the case of expression of transgenes, one of skill will recognize that the inserted polynucleotide sequence need not be identical but may be only substantially identical to a sequence of the gene from which it was derived. As is explained herein, these substantially identical variants are specifically covered by reference to a specific nucleic acid sequence. One example of an expression cassette is a polynucleotide construct that contains a polynucleotide sequence encoding a polypeptide for use in the invention operably linked to a promoter, e.g., its native promoter, where the expression cassette is introduced into a heterologous microorganism. In some embodiments, an expression cassette contains a polynucleotide sequence encoding a polypeptide of the invention where the polynucleotide that is targeted to a position in the genome of a microorganism such that expression of the polynucleotide sequence is driven by a promoter that is present in the microorganism.

As used herein, the term “fermentation composition” refers to a composition which contains genetically modified host cells and products, or metabolites produced by the genetically modified host cells. An example of a fermentation composition is a whole cell broth, which may be the entire contents of a vessel, including cells, aqueous phase, and compounds produced from the genetically modified host cells.

As used herein, the term “gene” refers to the segment of DNA involved in producing or encoding a polypeptide chain. It may include regions preceding and following the coding region (leader and trailer) as well as intervening sequences (introns) between individual coding segments (exons). Alternatively, the term “gene” can refer to the segment of DNA involved in producing or encoding a non-translated RNA, such as an rRNA, tRNA, gRNA, or micro RNA.

A “genetic pathway” or “biosynthetic pathway” as used herein refers to a set of at least two different coding sequences, where the coding sequences encode enzymes that catalyze different parts of a synthetic pathway to form a desired product (e.g., a cannabinoid). In a genetic pathway, a first encoded enzyme uses a substrate to make a first product which in turn is used as a substrate for a second encoded enzyme to make a second product. In some embodiments, the genetic pathway includes 3 or more members (e.g., 3, 4, 5, 6, 7, 8, 9, etc.), wherein the product of one encoded enzyme is the substrate for the next enzyme in the synthetic pathway.

As used herein, the term “genetic switch” refers to one or more genetic elements that allow controlled expression of enzymes, e.g., enzymes that catalyze the reactions of cannabinoid biosynthesis pathways. For example, a genetic switch can include one or more promoters operably linked to one or more genes encoding a biosynthetic enzyme, or one or more promoters operably linked to a transcriptional regulator which regulates expression one or more biosynthetic enzymes.

As used herein, the term “genetically modified” denotes a host cell that contains a heterologous nucleotide sequence. The genetically modified host cells described herein typically do not exist in nature.

As used herein, the term “heterologous” refers to what is not normally found in nature. The term “heterologous compound” refers to the production of a compound by a cell that does not normally produce the compound, or to the production of a compound at a level not normally produced by the cell. For example, a cannabinoid can be a heterologous compound.

The term “heterologous compound” refers to the production of a compound by a cell that does not normally produce the compound, or to the production of a compound at a level at which it is not normally produced by the cell.

As used herein, the phrase “heterologous enzyme” refers to an enzyme that is not normally found in a given cell in nature. The term encompasses an enzyme that is: (a) exogenous to a given cell (i.e., encoded by a nucleotide sequence that is not naturally present in the host cell or not naturally present in a given context in the host cell); and (b) naturally found in the host cell (e.g., the enzyme is encoded by a nucleotide sequence that is endogenous to the cell) but that is produced in an unnatural amount (e.g., greater or lesser than that naturally found) in the host cell.

A “heterologous genetic pathway” or a “heterologous biosynthetic pathway” as used herein refer to a genetic pathway that does not normally or naturally exist in an organism or cell.

The term “host cell” as used in the context of this invention refers to a microorganism, such as yeast, and includes an individual cell or cell culture contains a heterologous vector or heterologous polynucleotide as described herein. Host cells include progeny of a single host cell, and the progeny may not necessarily be completely identical (in morphology or in total DNA complement) to the original parent cell due to natural, accidental, or deliberate mutation and/or change. A host cell includes cells into which a recombinant vector or a heterologous polynucleotide of the invention has been introduced, including by transformation, transfection, and the like.

As used herein, the term “introducing” in the context of introducing a nucleic acid or protein into a host cell refers to any process that results in the presence of a heterologous nucleic acid or polypeptide inside the host cell. For example, the term encompasses introducing a nucleic acid molecule (e.g., a plasmid or a linear nucleic acid) that encodes the nucleic acid of interest (e.g., an RNA molecule) or polypeptide of interest and results in the transcription of the RNA molecule and translation of the polypeptide. The term also encompasses integrating the nucleic acid encoding the RNA molecule or polypeptide into the genome of a progenitor cell. The nucleic acid is then passed through subsequent generations to the host cell, so that, for example, a nucleic acid encoding an RNA-guided endonuclease is “pre-integrated” into the host cell genome. In some cases, introducing refers to translocation of a nucleic acid or polypeptide from outside the host cell to inside the host cell. Various methods of introducing nucleic acids, polypeptides and other biomolecules into host cells are contemplated, including but not limited to, electroporation, contact with nanowires or nanotubes, spheroplasting, PEG 1000-mediated transformation, biolistics, lithium acetate transformation, lithium chloride transformation, and the like.

As used herein, the term “medium” refers to culture medium and/or fermentation medium.

The terms “modified,” “recombinant,” and “engineered,” when used to modify a host cell described herein, refer to host cells or organisms that do not exist in nature, or express compounds, nucleic acids or proteins at levels that are not expressed by naturally occurring cells or organisms.

As used herein, the phrase “operably linked” refers to a functional linkage between nucleic acid sequences such that the linked promoter and/or regulatory region functionally controls expression of the coding sequence.

“Percent (%) sequence identity” with respect to a reference polynucleotide or polypeptide sequence is defined as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are within the capabilities of one of skill in the art, for example, using publicly available computer software such as BLAST, BLAST-2, or Megalign software. Those skilled in the art can determine appropriate parameters for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:

100 multiplied by(the fractionX/Y)

where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid

The terms “polynucleotide” and “nucleic acid” are used interchangeably and refer to a single or double-stranded polymer of deoxyribonucleotide or ribonucleotide bases read from the 5′ to the 3′ end. A nucleic acid as used in the present invention will generally contain phosphodiester bonds, although in some cases, nucleic acid analogs may be used that may have alternate backbones, comprising, e.g., phosphoramidate, phosphorothioate, phosphorodithioate, or O-methylphosphoroamidite linkages (see Eckstein, Oligonucleotides and Analogues: A Practical Approach, Oxford University Press); positive backbones; non-ionic backbones, and non-ribose backbones. Nucleic acids or polynucleotides may also include modified nucleotides that permit correct read-through by a polymerase. “Polynucleotide sequence” or “nucleic acid sequence” includes both the sense and antisense strands of a nucleic acid as either individual single strands or in a duplex. As will be appreciated by those in the art, the depiction of a single strand also defines the sequence of the complementary strand; thus, the sequences described herein also provide the complement of the sequence. Unless otherwise indicated, a particular nucleic acid sequence also implicitly encompasses variants thereof (e.g., degenerate codon substitutions) and complementary sequences, as well as the sequence explicitly indicated. The nucleic acid may be DNA, both genomic and cDNA, RNA or a hybrid, where the nucleic acid may contain combinations of deoxyribo- and ribo-nucleotides, and combinations of bases, including uracil, adenine, thymine, cytosine, guanine, inosine, xanthine hypoxanthine, isocytosine, isoguanine, etc. Nucleic acid sequences are presented in the 5′ to 3′ direction unless otherwise specified.

As used herein, the terms “polypeptide,” “peptide,” and “protein” are used interchangeably to refer to a polymer of amino acid residues. The terms encompass amino acid chains of any length, including full-length proteins, wherein the amino acid residues are linked by covalent peptide bonds.

As used herein, the term “production” generally refers to an amount of compound produced by a genetically modified host cell provided herein. In some embodiments, production is expressed as a yield of the compound by the host cell. In other embodiments, production is expressed as a productivity of the host cell in producing the compound.

As used herein, the term “productivity” refers to production of a compound by a host cell, expressed as the amount of non-catabolic compound produced (by weight) per amount of fermentation broth in which the host cell is cultured (by volume) over time (per hour).

As used herein, the term “promoter” refers to a synthetic or naturally-derived nucleic acid that is capable of activating, increasing, or enhancing expression of a DNA coding sequence, or inactivating, decreasing, or inhibiting expression of a DNA coding sequence. A promoter may contain one or more specific transcriptional regulatory sequences to further enhance or repress expression and/or to alter the spatial expression and/or temporal expression of the coding sequence. A promoter may be positioned 5′ (upstream) of the coding sequence under its control. A promoter may also initiate transcription in the downstream (3′) direction, the upstream (5′) direction, or be designed to initiate transcription in both the downstream (3′) and upstream (5′) directions. The distance between the promoter and a coding sequence to be expressed may be approximately the same as the distance between that promoter and the native nucleic acid sequence it controls. As is known in the art, variation in this distance may be accommodated without loss of promoter function. The term also includes a regulated promoter, which generally allows transcription of the nucleic acid sequence while in a permissive environment (e.g., microaerobic fermentation conditions, or the presence of maltose), but ceases transcription of the nucleic acid sequence while in a non-permissive environment (e.g., aerobic fermentation conditions, or in the absence of maltose). Promoters used herein can be constitutive, inducible, or repressible.

As used herein, the term “subtilisin” refers to extracellular serine endopeptidase isolated from the Bacillus genus. Examples of subtilisin enzymes include but are not limited to subtilisin Carlsberg from B. licheniformis, which is also known as subtilisin A, subtilopeptidase A, and alcalase Novo; subtilisin from B. amyloliquefaciens, which is also known as subtilisin BPN′, Nagarse, subtilisin B. sublilopeptidase B, subtilopeptidase C and bacterial proteinase Novo; subtilisin 147 or esperase from B. lentus; B. alcalophilus PB92; subtilisin 309 or savinase expressed in B. lentus, and subtilisin 168 also known as subtilisin E from B. subtilis strain 168.

The term “yield” refers to production of a compound by a host cell, expressed as the amount of compound produced per amount of carbon source consumed by the host cell, by weight.

### BRIEF DESCRIPTION OF THE DRAWINGS

FIGS. 1A-1C are a series of graphs showing the percent molar conversion of cannabigerolic acid (CBGA) to cannabigerol (CBG) (FIG. 1A), the percent molar yield of CBG (FIG. 1B), and the temperature of the bioreactor over time (FIG. 1C) in a high oleic sunflower oil overlay recovered without the Tergazyme® as a demulsification aid where the initial concentration of CBGA is either 32.5% or 2.3%. Details of each experimental method are provided in Example 1, below.

FIG. 2 is a schematic showing an exemplary cannabinoid purification process that may be used in conjunction with the compositions and methods of the disclosure.

FIG. 3 is a schematic showing an exemplary demulsification process that may be used in conjunction with the compositions and methods of the disclosure.

FIG. 4 is a schematic showing an exemplary evaporation process that may be used to concentrate a cannabinoid, for example, as part of a cannabinoid purification process described herein.

FIG. 5A is an image showing a 35% oil-in-water mixture that was treated with 1% Tergazyme® at a temperature of 55° C. for 60 minutes, as described in Example 1, below.

FIG. 5B is an image showing the whole cell broth and oil overlay before (right) and after (left) treatment with 1% Tergazyme® at a temperature of 55° C. for 60 minutes, as described in Example 1, below.

### DETAILED DESCRIPTION OF THE INVENTION

The present disclosure provides methods for purifying a cannabinoid from a fermentation composition. For example, using the compositions and methods described herein, a cannabinoid may be purified from a fermentation composition produced by culturing host cells genetically modified to express one or more enzyme of a cannabinoid biosynthetic pathway in a culture medium. The cannabinoid may be purified, for example, by contacting the fermentation composition with an enzymatic composition that includes a serine protease and subsequently isolating the cannabinoid from the fermentation composition and/or the enzymatic composition. The present disclosure also provides methods for decarboxylating a cannabinoid by contacting a fermentation composition including a population of host cells that are genetically modified to express one or more enzymes of a cannabinoid biosynthetic pathway with an enzymatic composition including a serine protease.

The enzymatic composition may be mixed for a time and at a temperature sufficient to allow for demulsification of the fermentation composition before the cannabinoid undergoes decarboxylation, and the cannabinoid may be recovered. The sections that follow describe exemplary methods for purifying a cannabinoid in further detail, as well as exemplary enzymes of a cannabinoid biosynthetic pathway that may be used in conjunction with the compositions and methods of the disclosure.

### Methods of Purifying a Cannabinoid

In an aspect, the disclosure provides a method for purifying a cannabinoid from a fermentation composition. The method may include culturing a population of host cells that are genetically modified to express one or more enzymes of a cannabinoid biosynthetic pathway in a culture medium and under conditions suitable for the host cells to produce the cannabinoid, thereby producing a fermentation composition; contacting the fermentation composition with an enzymatic composition comprising a serine protease; and recovering one or more cannabinoids from the fermentation composition and/or the enzymatic composition.

In another aspect, the disclosure provides a method of purifying a cannabinoid from a fermentation composition that has been produced by culturing a population of host cells that are genetically modified to express one or more enzymes of a cannabinoid biosynthetic pathway in a culture medium and under conditions suitable for the host cells to produce the cannabinoid; contacting the fermentation composition with an enzymatic composition comprising a serine protease, and recovering one or more cannabinoids from the fermentation composition and/or the enzymatic composition.

In some embodiments, the fermentation composition is separated into a supernatant and a pellet by solid-liquid centrifugation following the culturing of the population of host cells. In some embodiments, the fermentation is adjusted to a pH of about 7 before being contacted with the enzymatic composition. The enzymatic composition may have a final concentration of from about 0.5% (w/v) to about 3% (w/v) (e.g., 0.6% (w/v), 0.7% (w/V), 0.8% (w/v), 0.9% (w/V), 1% (w/v), 1.5% (w/v), 2% (w/v), and 2.5% (w/v) after contacting the fermentation composition with the enzymatic composition; for example, the fermentation composition may be contacted with the enzymatic composition at a concentration of 1% (w/v) final volume.

In some embodiments, the fermentation composition is mixed with the enzymatic composition for between 0.5 hours and 2 hours (e.g., between 30 minutes and 120 minutes, between 35 minutes and 105 minutes, between 40 minutes and 90 minutes, between 45 minutes and 75 minutes, and between 50 minutes and 60 minutes). For example, the fermentation composition may be mixed with the enzymatic composition for about 60 minutes.

In some embodiments, the fermentation composition undergoes liquid-liquid centrifugation after being contacted with the enzymatic composition. In some embodiments, the fermentation composition is passed through an evaporator after being contacted with the enzymatic composition. The fermentation composition may be passed through an evaporator more than once. For example, the fermentation composition may be passed through an evaporator twice. In some embodiments, the walls of the evaporator are heated to about 180° C. In some embodiments, the walls of the evaporator are heated to about 250° C. In certain embodiments, the condenser of the evaporator is heated to about 80° C. For example, the walls of the evaporator may be heated to about 180° C. and the condenser of the evaporator may be heated to about 80° C. the first time the fermentation composition is passed through the evaporator, and the walls of the evaporator may be heated to about 250° C. and the condenser of the evaporator may be heated to about 80° C. the second time the fermentation composition is passed through the evaporator. In some embodiments, the evaporate is a short-path evaporator (e.g., a wiped-film evaporator). In some embodiments, the fermentation composition is heated to about 180° C. or more for less than 5 minutes (e.g., less than 4 minutes 3 minutes, 2 minutes, and 1 minute); for example, fermentation composition may be heated to about 180° C. or more for less than 1 minute (e.g., less than 55 seconds, 50 seconds, 45 seconds, 40 seconds, 35 seconds, 30 seconds, 25 seconds, 20 seconds, 15 seconds, 10 seconds, and 5 seconds).

In some embodiments, the cannabinoid is recovered using crystallization after the fermentation solution is passed through the evaporator

The recovered cannabinoid may have a purity of between 50% and 100% (e.g., between 55% and 95%, between 60% and 90%, between 65% and 85%, and between 70% and 80% purity). For example, the recovered cannabinoid may have between 70% and 100% purity (e.g., between 75%, and 95%, and between 80% and 90% purity). The molar yield of the cannabinoid may be between 60% and 100% (e.g., between 65% and 95%, between 70% and 90%, and between 75% and 85%). For example, the molar yield may be between 90% and 100% (e.g., 91%, 92%, 93%, 94%, 95%, 96%, 97%, 98%, or 99%).

### Enzymatic Composition

In an aspect, the fermentation composition is contacted with an enzymatic composition including a serine protease. In some embodiments, the enzymatic composition includes between 0.003% and 20% serine protease by weight (e.g., between 0.003% and 15%, between 0.005% and 10%, between 0.007% and 7%, and between 0.01% and 5% serine protease by weight). In some embodiments, the enzymatic composition includes between 0.01% and 10% serine protease by weight (e.g., between 0.01% and 10%, between 0.02% and 9%, between 0.03% and 8%, between 0.04% and 7%, between 0.05% and 6%, between 0.06% and 5%, between 0.07% and 4%, between 0.08% and 3%, between 0.08% and 2%, between 0.09% and 1%, and between 0.1% and 1% serine protease by weight). In some embodiments, the enzymatic composition comprises between 0.01% and 5% by serine protease by weight (e.g., between 0.01% and 5%, between 0.05% and 4%, between 0.1% and 3%, between 0.5% and 2% serine protease by weight).

In some embodiments, the serine protease is a subtilisin. In some embodiments, the subtilisin is from Bacillus licheniformis. In some embodiments, the subtilisin is subtilisin Carlsberg. In some embodiments, the subtilisin has an amino acid sequence that is at least 90% (e.g., at least 91%, 92%, 93%, 94%, 95%, 96%, 97%, 98%, or 99%) identical to the amino acid sequence of SEQ ID NO: 1. In some embodiments, the subtilisin has an amino acid sequence that is at least 95% (e.g., at least 95%, 96%, 97%, 98%, or 99%) identical to the amino acid sequence of SEQ ID NO: 1. In some embodiments, the subtilisin has the amino acid sequence of SEQ ID NO: 1.

In some embodiments, the enzymatic composition includes an alkylaryl sulfonate salt. In some embodiments, the alkylaryl sulfonate includes a linear alkylaryl sulfonate salt. In some embodiments, the enzymatic composition includes a phosphate salt. In some embodiments, the enzymatic composition includes a carbonate salt. In some embodiments, the salt is a sodium salt.

In some embodiments, the enzymatic composition has a pH of between 8.5 and 11 (e.g., between pH 8.7 and pH 10.5, between pH 9.0 and pH 10, and between pH 9.2 and pH 9.7) in a 1% (w/v) solution. In some embodiments, the enzymatic composition has a pH of about 9.5 in a 1% (w/v) solution. In some embodiments, the enzymatic composition is Tergazyme®.

### Cannabinoid Pathway

In an aspect, the host cell includes one or more nucleic acids encoding one or more enzymes of a heterologous genetic pathway that produces a cannabinoid or a precursor of a cannabinoid. The cannabinoid biosynthetic pathway may begin with hexanoic acid as the substrate for an acyl activating enzyme (AAE) to produce hexanoyl-CoA, which is used as the substrate of a tetraketide synthase (TKS) to produce tetraketide-CoA, which is used by an olivetolic acid cyclase (OAC) to produce olivetolic acid, which is then used to produce a cannabigerolic acid by a geranyl pyrophosphate (GPP) synthase and a cannabigerolic acid synthase (CBGaS). In some embodiments, the cannabinoid precursor that is produced is a substrate in the cannabinoid pathway (e.g., hexanoate or olivetolic acid). In some embodiments, the precursor is a substrate for an AAE, a TKS, an OAC, a CBGaS, or a GPP synthase. In some embodiments, the precursor, substrate, or intermediate in the cannabinoid pathway is hexanoate, olivetol, or olivetolic acid. In some embodiments, the precursor is hexanoate. In some embodiments, the host cell does not contain the precursor, substrate or intermediate in an amount sufficient to produce the cannabinoid or a precursor of the cannabinoid. In some embodiments, the host cell does not contain hexanoate at a level or in an amount sufficient to produce the cannabinoid in an amount over 10 mg/L. In some embodiments, the heterologous genetic pathway encodes at least one enzyme selected from the group consisting of an AAE, a TKS, an OAC, a CBGaS, or a GPP synthase. In some embodiments, the genetically modified host cell includes an AAE, TKS, OAC, CBGaS, and a GPP synthase. The cannabinoid pathway is described in Keasling et al., U.S. Pat. No. 10,563,211, the disclosure of which is incorporated herein by reference.

### Acyl Activating Enzymes

Some embodiments concern a host cell that includes a heterologous AAE such that the host cell is capable of producing a cannabinoid. The AAE may be from Cannabis sativa or may be an enzyme from another plant or fungal source which has been shown to have AAE activity in the cannabinoid biosynthetic pathway, resulting in the production of the cannabinoid precursor olivetolic acid. In some embodiments, the host cell contains a heterologous nucleic acid that encodes an AAE having an amino acid sequence that is at least 90% identical to the amino acid sequence of any one of SEQ ID NO: 2-25 (e.g., an amino acid sequence that is 90%, 91%, 92%, 93%, 94%, 95%, 96%, 97%, 98%, 99%, or 100% identical to any one of SEQ ID NO: 2-25). For example, the AAE may have an amino acid sequence that is at least 90% identical to the amino acid sequence of any one of SEQ ID NO: 2-25. In some embodiments, the AAE has an amino acid sequence that is at least 95% identical to the amino acid sequence of any one of SEQ ID NO: 2-25 (e.g., an amino acid sequence that is 95%, 96%, 97%, 98%, 99%, or 100% identical to any one of SEQ ID NO: 2-25). In some embodiments, the AAE has the amino acid sequence of any one of SEQ ID NO: 2-25. In some embodiments, the host cell contains a heterologous nucleic acid that encodes an AAE having an amino acid sequence that is at least 90% identical to the amino acid sequence of any one of SEQ ID NO: 2-14 (e.g., an amino acid sequence that is 90%, 91%, 92%, 93%, 94%, 95%, 96%, 97%, 98%, 99%, or 100% identical to any one of SEQ ID NO: 2-14). In some embodiments, the AAE has an amino acid sequence that is at least 95% identical to the amino acid sequence of any one of SEQ ID NO: 2-14 (e.g., an amino acid sequence that is 95%, 96%, 97%, 98%, 99%, or 100% identical to any one of SEQ ID NO: 2-14). In some embodiments, the AAE has the amino acid sequence of any one of SEQ ID NO: 2-14. In some embodiments, the host cell contains a heterologous nucleic acid that encodes an AAE having an amino acid sequence that is at least 90% identical to the amino acid sequence of any one of SEQ ID NO: 2-6 (e.g., an amino acid sequence that is 90%, 91%, 92%, 93%, 94%, 95%, 96%, 97%, 98%, 99%, or 100% identical to any one of SEQ ID NO: 2-6). In some embodiments, the AAE has an amino acid sequence that is at least 95% identical to the amino acid sequence of any one of SEQ ID NO: 2-6 (e.g., an amino acid sequence that is 95%, 96%, 97%, 98%, 99%, or 100% identical to any one of SEQ ID NO: 2-6). In some embodiments, the AAE has the amino acid sequence of any one of SEQ ID NO: 2-6.

### Tetraketide Synthase Enzymes

Some embodiments concern a host cell that includes a heterologous TKS such that the host cell is capable of producing a cannabinoid. A TKS uses the hexanoyl-CoA precursor to generate tetraketide-CoA. The TKS may be from Cannabis sativa or may be an enzyme from another plant or fungal source which has been shown to have TKS activity in the cannabinoid biosynthetic pathway, resulting in the production of the cannabinoid precursor olivetolic acid. In some embodiments, the host cell contains a heterologous nucleic acid that encodes a TKS having an amino acid sequence that is at least 90% identical to the amino acid sequence of any one of SEQ ID NO: 26-60 (e.g., an amino acid sequence that is 90%, 91%, 92%, 93%, 94%, 95%, 96%, 97%, 98%, 99%, or 100% identical to any one of SEQ ID NO: 26-60). In some embodiments, the TKS has an amino acid sequence that is at least 95% identical to the amino acid sequence of any one of SEQ ID NO: 26-60 (e.g., an amino acid sequence that is 95%, 96%, 97%, 98%, 99%, or 100% identical to any one of SEQ ID NO: 26-60). In some embodiments, the TKS has the amino acid sequence of any one of SEQ ID NO: 26-60. In some embodiments, the host cell contains a heterologous nucleic acid that encodes a TKS having an amino acid sequence that is at least 90% identical to the amino acid sequence of any one of SEQ ID NO: 26-29 (e.g., an amino acid sequence that is 90%, 91%, 92%, 93%, 94%, 95%, 96%, 97%, 98%, 99%, or 100% identical to any one of SEQ ID NO: 26-29). In some embodiments, the TKS has an amino acid sequence that is at least 95% identical to the amino acid sequence of any one of SEQ ID NO: 26-29 (e.g., an amino acid sequence that is 95%, 96%, 97%, 98%, 99%, or 100% identical to any one of SEQ ID NO: 26-29). In some embodiments, the TKS has the amino acid sequence of any one of SEQ ID NO: 26-29. In some embodiments, the host cell contains a heterologous nucleic acid that encodes a TKS having an amino acid sequence that is at least 90% identical to the amino acid sequence of SEQ ID NO: 26 (e.g., an amino acid sequence that is 90%, 91%, 92%, 93%, 94%, 95%, 96%, 97%, 98%, 99%, or 100% identical to SEQ ID NO: 26). In some embodiments, the TKS has an amino acid sequence that is at least 95% identical to the amino acid sequence of SEQ ID NO: 26 (e.g., an amino acid sequence that is 95%, 96%, 97%, 98%, 99%, or 100% identical to SEQ ID NO: 26). In some embodiments, the TKS has the amino acid sequence of SEQ ID NO: 26.

### Cannabigerolic Acid Synthases

Some embodiments concern a host cell that includes a heterologous CBGaS such that the host cell is capable of producing a cannabinoid. A CBGaS uses the olivetolic acid precursor and GPP precursor to generate cannabigerolic acid. The CBGaS may be from Cannabis sativa or may be an enzyme from another plant or fungal source which has been shown to have CBGaS activity in the cannabinoid biosynthetic pathway, resulting in the production of the cannabinoid cannabigerolic acid. In some embodiments, the host cell contains a heterologous nucleic acid that encodes a CBGaS having an amino acid sequence that is at least 90% identical to the amino acid sequence of any one of SEQ ID NO: 61-65 (e.g., an amino acid sequence that is 90%, 91%, 92%, 93%, 94%, 95%, 96%, 97%, 98%, 99%, or 100% identical to any one of SEQ ID NO: 61-65). In some embodiments, the CBGaS has an amino acid sequence that is at least 95% identical to the amino acid sequence of any one of SEQ ID NO: 61-65 (e.g., an amino acid sequence that is 95%, 96%, 97%, 98%, 99%, or 100% identical to any one of SEQ ID NO: 61-65). In some embodiments, the CBGaS has the amino acid sequence of any one of SEQ ID NO: 61-65.

### Geranyl Pyrophosphate Synthase

Some embodiments concern a host cell that includes a heterologous GPP synthase such that the host cell is capable of producing a cannabinoid. A GPP synthase uses the product of the isoprenoid biosynthesis pathway precursor to generate cannabigerolic acid together with a prenyltransferase enzyme. The GPP synthase may be from Cannabis sativa or may be an enzyme from another plant or bacterial source which has been shown to have GPP synthase activity in the cannabinoid biosynthetic pathway, resulting in the production of the cannabinoid cannabigerolic acid. In some embodiments, the host cell contains a heterologous nucleic acid that encodes a GPP synthase having an amino acid sequence that is at least 90% identical to the amino acid sequence of any one of SEQ ID NO: 66-71 (e.g., an amino acid sequence that is 90%, 91%, 92%, 93%, 94%, 95%, 96%, 97%, 98%, 99%, or 100% identical to any one of SEQ ID NO: 66-71). In some embodiments, the GPP synthase has an amino acid sequence that is at least 95% identical to the amino acid sequence of any one of SEQ ID NO: 66-71 (e.g., an amino acid sequence that is 95%, 96%, 97%, 98%, 99%, or 100% identical to any one of SEQ ID NO: 66-71). In some embodiments, GPP synthase has the amino acid sequence of any one of SEQ ID NO: 66-71. In some embodiments, the host cell contains a heterologous nucleic acid that encodes a GPP synthase having an amino acid sequence that is at least 90% identical to the amino acid sequence of SEQ ID NO: 66 (e.g., an amino acid sequence that is 90%, 91%, 92%, 93%, 94%, 95%, 96%, 97%, 98%, 99%, or 100% identical to SEQ ID NO: 66). In some embodiments, the GPP synthase has an amino acid sequence that is at least 95% identical to the amino acid sequence of SEQ ID NO: 66 (e.g., an amino acid sequence that is 95%, 96%, 97%, 98%, 99%, or 100% identical to SEQ ID NO: 66). In some embodiments, the GPP synthase has the amino acid sequence of SEQ ID NO: 66.

### Additional Enzymes

The host cell may further express other heterologous enzymes in addition to the AAE, TKS, CBGaS, and/or GPP synthase. For example, in some embodiments, the host cell may include a heterologous nucleic acid that encodes at least one enzyme from the mevalonate biosynthetic pathway. Enzymes which make up the mevalonate biosynthetic pathway may include but are not limited to an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase. In some embodiments, the host cell includes a heterologous nucleic acid that encodes the acetyl-CoA thiolase, the HMG-COA synthase, the HMG-COA reductase, the mevalonate kinase, the phosphomevalonate kinase, the mevalonate pyrophosphate decarboxylase, and the IPP:DMAPP isomerase of the mevalonate biosynthesis pathway.

In some embodiments, the host cell may include an olivetolic acid cyclase (OAC) as part of the cannabinoid biosynthetic pathway. The OAC may have an amino acid sequence that is at least 90% (e.g., at least 91%, 92%, 93%, 94%, 95%, 96%, 97%, 98%, or 99%) identical to SEQ ID NO: 72. The OAC may have an amino acid sequence that is at least 95% (e.g., at least 96%, 97%, 98%, or 99%) identical to SEQ ID NO: 72. In some embodiments, the OAC has an amino acid sequence of SEQ ID NO: 72.

In some embodiments, the host cell further includes one or more heterologous nucleic acids that each, independently, encode an acetyl-CoA synthase, and/or an aldehyde dehydrogenase, and/or a pyruvate decarboxylase. In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 73. In some embodiments, the acetyl-CoA synthase has an amino acid sequence that is at least 95% (e.g., at least 96%, 97%, 98%, or 99%) identical to the amino acid sequence of SEQ ID NO: 73. In some embodiments, the acetyl-CoA synthase has the amino acid sequence of SEQ ID NO: 73.

In some embodiments, the acetyl-CoA synthase has an amino acid sequence that is at least 90% (e.g., at least 91%, 92%, 93%, 94%, 95%, 96%, 97%, 98%, or 99%) identical to the amino acid sequence of SEQ ID NO: 74. In some embodiments, the acetyl-CoA synthase has an amino acid sequence that is at least 95% (e.g., at least 96%, 97%, 98%, or 99%) identical to the amino acid sequence of SEQ ID NO: 74. In some embodiments, the acetyl-CoA synthase has the amino acid sequence of SEQ ID NO: 74.

In some embodiments, the aldehyde dehydrogenase has an amino acid sequence that is at least 90% (e.g., at least 91%, 92%, 93%, 94%, 95%, 96%, 97%, 98%, or 99%) identical to the amino acid sequence of SEQ ID NO: 75. In some embodiments, the aldehyde dehydrogenase has an amino acid sequence that is at least 95% (e.g., at least 96%, 97%, 98%, or 99%) identical to the amino acid sequence of SEQ ID NO: 75. In some embodiments, the aldehyde dehydrogenase synthase has the amino acid sequence of SEQ ID NO: 75.

In some embodiments, the pyruvate decarboxylase has an amino acid sequence that is at least 90% (e.g., at least 91%, 92%, 93%, 94%, 95%, 96%, 97%, 98%, or 99%) identical to the amino acid sequence of SEQ ID NO: 76. In some embodiments, the pyruvate decarboxylase has an amino acid sequence that is at least 95% (e.g., at least 96%, 97%, 98%, or 99%) identical to the amino acid sequence of SEQ ID NO: 76. In some embodiments, the pyruvate decarboxylase has the amino acid sequence of SEQ ID NO: 76.

In some embodiments, the host cell contains a heterologous nucleic acid encoding an aceto-CoA carboxylase (ACC). In some embodiments, the heterologous nucleic acid encodes a ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78.

In some embodiments, the host cell contains a heterologous nucleic acid encoding an ACC and an acetoacetyl-CoA synthase (AACS) instead of a heterologous nucleic acid encoding an acetyl-CoA thiolase. In some embodiments, the heterologous nucleic acid encodes an ACC having an amino acid sequence that is at least 90% identical to the amino acid sequence of SEQ ID NO: 78 (e.g., an amino acid sequence that is 90%, 91%, 92%, 93%, 94%, 95%, 96%, 97%, 98%, 99%, or 100% identical to SEQ ID NO: 78). In some embodiments, the ACC has an amino acid sequence that is at least 95% identical to the amino acid sequence of SEQ ID NO: 78 (e.g., an amino acid sequence that is 95%, 96%, 97%, 98%, 99%, or 100% identical to SEQ ID NO: 78). In some embodiments, the ACC has the amino acid sequence of SEQ ID NO: 78. In some embodiments, the heterologous nucleic acid encodes an AACS having an amino acid sequence that is at least 90% identical to the amino acid sequence of SEQ ID NO: 77 (e.g., an amino acid sequence that is 90%, 91%, 92%, 93%, 94%, 95%, 96%, 97%, 98%, 99%, or 100% identical to SEQ ID NO: 77). In some embodiments, the AACS has an amino acid sequence that is at least 95% identical to the amino acid sequence of SEQ ID NO: 77 (e.g., an amino acid sequence that is 95%, 96%, 97%, 98%, 99%, or 100% identical to SEQ ID NO: 77). In some embodiments, the AACS has the amino acid sequence of SEQ ID NO: 77.

Due to the inherent degeneracy of the genetic code, other polynucleotides which encode substantially the same or functionally equivalent polypeptides can also be used to clone and express the polynucleotides encoding the protein components of the heterologous genetic pathway described herein.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, in a process sometimes called “codon optimization” or “controlling for species codon bias.”

Optimized coding sequences containing codons preferred by a particular prokaryotic or eukaryotic host (Murray et al., 1989, Nucl Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al., 1996, Nucl Acids Res. 24:216-8).

Those of skill in the art will recognize that, due to the degenerate nature of the genetic code, a variety of DNA molecules differing in their nucleotide sequences can be used to encode a given enzyme of the disclosure. Any one of the polypeptide sequences disclosed herein may be encoded by DNA molecules of any sequence that encode the amino acid sequences of the polypeptides and proteins of the enzymes utilized in the methods of the disclosure. In a similar fashion, a polypeptide can typically tolerate one or more amino acid substitutions, deletions, and insertions in its amino acid sequence without loss or significant loss of a desired activity. The disclosure includes such polypeptides with different amino acid sequences than the specific proteins described herein so long as the modified or variant polypeptides have the enzymatic anabolic or catabolic activity of the reference polypeptide. Furthermore, the amino acid sequences encoded by the DNA sequences shown herein merely illustrate embodiments of the disclosure.

In addition, homologs of enzymes that may be used in conjunction with the compositions and methods provided herein are encompassed by the disclosure. In some embodiments, two proteins (or a region of the proteins) are substantially homologous when the amino acid sequences have at least about 30%, 40%, 50% 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (e.g., Pearson W. R., 1994, Methods in Mol Biol 25:365-89).

The following six groups each contain amino acids that are conservative substitutions for one another: 1) Serine(S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. A typical algorithm used to compare a molecule sequence to a database containing a large number of sequences from different organisms is the computer program BLAST. When searching a database containing sequences from a large number of different organisms, it is typical to compare amino acid sequences.

Furthermore, any of the genes encoding the foregoing enzymes (or any others mentioned herein (or any of the regulatory elements that control or modulate expression thereof)) may be optimized by genetic/protein engineering techniques, such as directed evolution or rational mutagenesis, which are known to those of ordinary skill in the art. Such action allows those of ordinary skill in the art to optimize the enzymes for expression and activity in a host cell, for example, a yeast.

In addition, genes encoding these enzymes can be identified from other fungal and bacterial species and can be expressed in the host cell. A variety of organisms could serve as sources for these enzymes, including, but not limited to, Saccharomyces spp., including S. cerevisiae and S. uvarum, Kluyveromyces spp., including K. thermotolerans, K. lactis, and K. marxianus, Pichia spp., Hansenula spp., including H. polymorphs, Candida spp., Trichosporon spp., Yamadazyma spp., including Y. spp. stipitis, Torulaspora pretoriensis, Issatchenkia orientalis, Schizosaccharomyces spp., including S. pombe, Cryptococcus spp., Aspergillus spp., Neurospora spp., or Ustilago spp. Sources of genes from anaerobic fungi include, but are not limited to, Piromyces spp., Orpinomyces spp., or Neocallimastix spp. Sources of prokaryotic enzymes that are useful include, but are not limited to, Escherichia coli, Zymomonas mobilis, Staphylococcus aureus, Bacillus spp., Clostridium spp., Corynebacterium spp., Pseudomonas spp., Lactococcus spp., Enterobacter spp., and Salmonella spp.

Techniques known to those skilled in the art may be suitable to identify additional homologous genes and homologous enzymes. Generally, analogous genes and/or analogous enzymes can be identified by functional analysis and will have functional similarities. Techniques known to those skilled in the art may be suitable to identify analogous genes and analogous enzymes. For example, to identify homologous or analogous ADA genes, proteins, or enzymes, techniques may include, but are not limited to, cloning a gene by PCR using primers based on a published sequence of an ADA gene/enzyme or by degenerate PCR using degenerate primers designed to amplify a conserved region among ADA genes. Further, one skilled in the art can use techniques to identify homologous or analogous genes, proteins, or enzymes with functional homology or similarity. Techniques include examining a cell or cell culture for the catalytic activity of an enzyme through in vitro enzyme assays for said activity (e.g. as described herein or in Kiritani, K., Branched-Chain Amino Acids Methods Enzymology, 1970), then isolating the enzyme with said activity through purification, determining the protein sequence of the enzyme through techniques such as Edman degradation, design of PCR primers to the likely nucleic acid sequence, amplification of said DNA sequence through PCR, and cloning of said nucleic acid sequence. To identify homologous or similar genes and/or homologous or similar enzymes, analogous genes and/or analogous enzymes or proteins, techniques also include comparison of data concerning a candidate gene or enzyme with databases such as BRENDA, KEGG, JGI Phyzome v12.1, BLAST, NCBI RefSeq, UniProt KB, or MetaCYC Protein annotations in the UniProt Knowledgebase may also be used to identify enzymes which have a similar function in addition to the National Center for Biotechnology Information RefSeq database. The candidate gene or enzyme may be identified within the above-mentioned databases in accordance with the teachings herein.

### Modified Host Cells

In one aspect, provided herein are host cells comprising at least one enzyme of the cannabinoid biosynthetic pathway (e.g., AAE, TKS, CBGaS, and GPP synthase). In some embodiments, the cannabinoid biosynthetic pathway contains a genetic regulatory element, such as a nucleic acid sequence, which is regulated by an exogenous agent. In some embodiments, the exogenous agent acts to regulate expression of the heterologous genetic pathway. Thus, in some embodiments, the exogenous agent can be a regulator of gene expression.

In some embodiments, the exogenous agent can be used as a carbon source by the host cell. For example, the same exogenous agent can both regulate production of a cannabinoid and provide a carbon source for growth of the host cell. In some embodiments, the exogenous agent is galactose. In some embodiments, the exogenous agent is maltose.

In some embodiments, the genetic regulatory element is a nucleic acid sequence, such as a promoter.

In some embodiments, the genetic regulatory element is a galactose-responsive promoter. In some embodiments, galactose positively regulates expression of the cannabinoid biosynthetic pathway, thereby increasing production of the cannabinoid. In some embodiments, the galactose-responsive promoter is a GAL1 promoter. In some embodiments, the galactose-responsive promoter is a GAL10 promoter. In some embodiments, the galactose-responsive promoter is a GAL2, GAL3, or GAL7 promoter. In some embodiments, heterologous genetic pathway contains the galactose-responsive regulatory elements described in Westfall et al. (PNAS (2012) vol. 109: E111-118). In some embodiments, the host cell lacks the gal1 gene and is unable to metabolize galactose, but galactose can still induce galactose-regulated genes.

### Table 1: Exemplary GAL Promoter Sequences

Promoter Sequence
pGAL1 SEQ ID NO: 79
pGAL10 SEQ ID NO: 80
pGAL2 SEQ ID NO: 81
pGAL3 SEQ ID NO: 82
pGAL7 SEQ ID NO: 83
pGAL4 SEQ ID NO: 84

In some embodiments, the galactose regulation system used to control expression of AAE, and/or, TKS, and/or CBGaS, and/or GPP synthase is re-configured such that it is no longer induced by the presence of galactose. Instead, the genes (e.g., AAE, TKS, CBGaS, or GPP synthase) will be expressed unless repressors, which may be maltose in some strains, are present in the medium.

In some embodiments, the genetic regulatory element is a maltose-responsive promoter. In some embodiments, maltose negatively regulates expression of the cannabinoid biosynthetic pathway, thereby decreasing production of the cannabinoid. In some embodiments, the maltose-responsive promoter is selected from the group consisting of pMAL1, pMAL2, pMAL11, pMAL12, pMAL31 and pMAL32. The maltose genetic regulatory element can be designed to both activate expression of some genes and repress expression of others, depending on whether maltose is present or absent in the medium. Maltose regulation of gene expression and maltose-responsive promoters are described in U.S. Patent Publication 2016/0177341, which is hereby incorporated by reference. Genetic regulation of maltose metabolism is described in Novak et al., “Maltose Transport and Metabolism in S. cerevisiae,” Food Technol. Biotechnol. 42 (3) 213-218 (2004).

### Table 2: Exemplary MAL Promoter Sequences

Promoter Sequence
pMAL1 SEQ ID NO: 85
pMAL2 SEQ ID NO: 86
pMAL11 SEQ ID NO: 87
pMAL12 SEQ ID NO: 88
pMAL31 SEQ ID NO: 89
pMAL32 SEQ ID NO: 90

In some embodiments, the heterologous genetic pathway is regulated by a combination of the maltose and galactose regulons.

In some embodiments, the recombinant host cell does not contain, or expresses a very low level of (for example, an undetectable amount), a precursor (e.g., hexanoic acid) required to make the cannabinoid. In some embodiments, the precursor (e.g., hexanoic acid) is a substrate of an enzyme in the cannabinoid biosynthetic pathway.

### Yeast Strains

In some embodiments, yeasts useful in the present methods include yeasts that have been deposited with microorganism depositories (e.g. IFO, ATCC, etc.) and belong to the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, chizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In some embodiments, the strain is Saccharomyces cerevisiae, Pichia pastoris, Schizosaccharomyces pombe, Dekkera bruxellensis, Kluyveromyces lactis (previously called Saccharomyces lactis), Kluveromyces marxianus, Arxula adeninivorans, or Hansenula polymorphs (now known as Pichia angusta). In some embodiments, the host microbe is a strain of the genus Candida, such as Candida lipolytica, Candida guilliermondii, Candida krusei, Candida pseudotropicalis, or Candida utilis.

In a particular embodiment, the strain is Saccharomyces cerevisiae. In some embodiments, the host is a strain of Saccharomyces cerevisiae selected from the group consisting of Baker's yeast, CEN.PK, CEN.PK2, CBS 7959, CBS 7960, CBS 7961, CBS 7962, CBS 7963, CBS 7964, IZ-1904, TA, BG-1, CR-1, SA-1, M-26, Y-904, PE-2, PE-5, VR-1, BR-1, BR-2, ME-2, VR-2, MA-3, MA-4, CAT-1, CB-1, NR-1, BT-1, and AL-1. In some embodiments, the strain of Saccharomyces cerevisiae is CEN.PK.

In some embodiments, the strain is a microbe that is suitable for industrial fermentation. In particular embodiments, the microbe is conditioned to subsist under high solvent concentration, high temperature, expanded substrate utilization, nutrient limitation, osmotic stress due to sugar and salts, acidity, sulfite and bacterial contamination, or combinations thereof, which are recognized stress conditions of the industrial fermentation environment.

### Mixtures

In another aspect, provided are mixtures including a fermentation composition produced by host cells that are genetically modified to express one or more enzymes of a cannabinoid biosynthetic pathway in a culture medium and under conditions suitable for the host cells to produce the cannabinoid and an enzymatic composition including a serine protease. In some embodiments, the serine protease is a subtilisin from Bacillus licheniformis. In some embodiments, the enzymatic composition includes sodium linear alkylaryl sulfonates, phosphates, and carbonates. In some embodiments, the host cells include one or more heterologous nucleic acids that each, independently, encode an AAE, and/or a TKS, and/or a CBGaS, and/or GPP synthase. In some embodiments, the host cell further includes one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

### Methods of Making the Host Cells

In another aspect, provided are methods of making the modified host cells described herein. In some embodiments, the methods include transforming a host cell with the heterologous nucleic acid constructs described herein which encode the proteins expressed by a heterologous genetic pathway described herein. Methods for transforming host cells are described in “Laboratory Methods in Enzymology: DNA”, Edited by Jon Lorsch, Volume 529, (2013); and U.S. Pat. No. 9,200,270 to Hsieh, Chung-Ming, et al., and references cited therein.

### Methods for Producing a Cannabinoid

In another aspect, methods are provided for producing a cannabinoid are described herein. In some embodiments, the method decreases expression of the cannabinoid. In some embodiments, the method includes culturing a host cell comprising at least one enzyme of the cannabinoid biosynthetic pathway described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in less than 0.001 mg/L of cannabinoid or a precursor thereof.

In some embodiments, the method is for decreasing expression of a cannabinoid or precursor thereof. In some embodiments, the method includes culturing a host cell comprising an AAE, and/or a TKS, and/or a CBGaS, and/or a GPP synthase described herein in a medium comprising an exogenous agent, wherein the exogenous agent decreases the expression of the cannabinoid. In some embodiments, the exogenous agent is maltose. In some embodiments, the exogenous agent is maltose. In some embodiments, the method results in the production of less than 0.001 mg/L of a cannabinoid or a precursor thereof.

In some embodiments, the method increases the expression of a cannabinoid. In some embodiments, the method includes culturing a host cell comprising an AAE, and/or a TKS, and/or a CBGaS, and/or a GPP synthase described herein in a medium comprising the exogenous agent, wherein the exogenous agent increases expression of the cannabinoid. In some embodiments, the exogenous agent is galactose. In some embodiments, the method further includes culturing the host cell with the precursor or substrate required to make the cannabinoid.

In some embodiments, the method increases the expression of a cannabinoid product or precursor thereof. In some embodiments, the method includes culturing a host cell comprising a heterologous cannabinoid pathway described herein in a medium comprising an exogenous agent, wherein the exogenous agent increases the expression of the cannabinoid or a precursor thereof. In some embodiments, the exogenous agent is galactose. In some embodiments, the method further includes culturing the host cell with a precursor or substrate required to make the cannabinoid or precursor thereof. In some embodiments, the precursor required to make the cannabinoid or precursor thereof is hexanoate. In some embodiments, the combination of the exogenous agent and the precursor or substrate required to make the cannabinoid or precursor thereof produces a higher yield of cannabinoid than the exogenous agent alone.

In some embodiments, the cannabinoid or a precursor thereof is cannabidiolic acid (CBDA), cannabidiol (CBD), cannabigerolic acid (CBGA), or cannabigerol (CBG).

### Culture and Fermentation Methods

Materials and methods for the maintenance and growth of microbial cultures are well known to those skilled in the art of microbiology or fermentation science (see, for example, Bailey et al., Biochemical Engineering Fundamentals, second edition, McGraw Hill, New York, 1986). Consideration must be given to appropriate culture medium, pH, temperature, and requirements for aerobic, microaerobic, or anaerobic conditions, depending on the specific requirements of the host cell, the fermentation, and the process.

The methods of producing cannabinoids provided herein may be performed in a suitable culture medium in a suitable container, including but not limited to a cell culture plate, a flask, or a fermentor. Further, the methods can be performed at any scale of fermentation known in the art to support industrial production of microbial products. Any suitable fermentor may be used including a stirred tank fermentor, an airlift fermentor, a bubble fermentor, or any combination thereof. In particular embodiments utilizing Saccharomyces cerevisiae as the host cell, strains can be grown in a fermentor as described in detail by Kosaric, et al, in Ullmann's Encyclopedia of Industrial Chemistry, Sixth Edition, Volume 12, pages 398-473, Wiley-VCH Verlag Gmbh & Co. KDaA, Weinheim, Germany.

In some embodiments, the culture medium is any culture medium in which a genetically modified microorganism capable of producing a heterologous product can subsist, i.e., maintain growth and viability. In some embodiments, the culture medium is an aqueous medium comprising assimilable carbon, nitrogen, and phosphate sources. Such a medium can also include appropriate salts, minerals, metals, and other nutrients. In some embodiments, the carbon source and each of the essential cell nutrients are added incrementally or continuously to the fermentation medium, and each required nutrient is maintained at essentially the minimum level needed for efficient assimilation by growing cells, for example, in accordance with a predetermined cell growth curve based on the metabolic or respiratory function of the cells which convert the carbon source to a biomass.

Suitable conditions and suitable medium for culturing microorganisms are well known in the art. In some embodiments, the suitable medium is supplemented with one or more additional agents, such as, for example, an inducer (e.g., when one or more nucleotide sequences encoding a gene product are under the control of an inducible promoter), a repressor (e.g., when one or more nucleotide sequences encoding a gene product are under the control of a repressible promoter), or a selection agent (e.g., an antibiotic to select for microorganisms comprising the genetic modifications).

In some embodiments, the carbon source is a monosaccharide (simple sugar), a disaccharide, a polysaccharide, a non-fermentable carbon source, or one or more combinations thereof. Non-limiting examples of suitable monosaccharides include glucose, galactose, mannose, fructose, ribose, and combinations thereof. Non-limiting examples of suitable disaccharides include sucrose, lactose, maltose, trehalose, cellobiose, and combinations thereof. Non-limiting examples of suitable polysaccharides include starch, glycogen, cellulose, chitin, and combinations thereof. Non-limiting examples of suitable non-fermentable carbon sources include acetate and glycerol.

The concentration of a carbon source, such as glucose or sucrose, in the culture medium should promote cell growth, but not be so high as to repress growth of the microorganism used. Typically, cultures are run with a carbon source, such as glucose or sucrose, being added at levels to achieve the desired level of growth and biomass. Production of cannabinoids may also occur in these culture conditions, but at undetectable levels (with detection limits being about <0.1 g/l). In other embodiments, the concentration of a carbon source, such as glucose or sucrose, in the culture medium is greater than about 1 g/L, preferably greater than about 2 g/L, and more preferably greater than about 5 g/L. In addition, the concentration of a carbon source, such as glucose or sucrose, in the culture medium is typically less than about 100 g/L, preferably less than about 50 g/L, and more preferably less than about 20 g/L. It should be noted that references to culture component concentrations can refer to both initial and/or ongoing component concentrations. In some cases, it may be desirable to allow the culture medium to become depleted of a carbon source during culture.

Sources of assimilable nitrogen that can be used in a suitable culture medium include, but are not limited to, simple nitrogen sources, organic nitrogen sources and complex nitrogen sources. Such nitrogen sources include anhydrous ammonia, ammonium salts and substances of animal, vegetable, and/or microbial origin. Suitable nitrogen sources include, but are not limited to, protein hydrolysates, microbial biomass hydrolysates, peptone, yeast extract, ammonium sulfate, urea, and amino acids. Typically, the concentration of the nitrogen sources, in the culture medium is greater than about 0.1 g/L, preferably greater than about 0.25 g/L, and more preferably greater than about 1.0 g/L. Beyond certain concentrations, however, the addition of a nitrogen source to the culture medium is not advantageous for the growth of the microorganisms. As a result, the concentration of the nitrogen sources, in the culture medium is less than about 20 g/L, preferably less than about 10 g/L and more preferably less than about 5 g/L. Further, in some instances it may be desirable to allow the culture medium to become depleted of the nitrogen sources during culture.

The effective culture medium can contain other compounds such as inorganic salts, vitamins, trace metals, or growth promoters. Such other compounds can also be present in carbon, nitrogen, or mineral sources in the effective medium or can be added specifically to the medium.

The culture medium can also contain a suitable phosphate source. Such phosphate sources include both inorganic and organic phosphate sources. Preferred phosphate sources include, but are not limited to, phosphate salts such as mono or dibasic sodium and potassium phosphates, ammonium phosphate, and mixtures thereof. Typically, the concentration of phosphate in the culture medium is greater than about 1.0 g/L, preferably greater than about 2.0 g/L, and more preferably greater than about 5.0 g/L. Beyond certain concentrations, however, the addition of phosphate to the culture medium is not advantageous for the growth of the microorganisms. Accordingly, the concentration of phosphate in the culture medium is typically less than about 20 g/L, preferably less than about 15 g/L, and more preferably less than about 10 g/L.

A suitable culture medium can also include a source of magnesium, preferably in the form of a physiologically acceptable salt, such as magnesium sulfate heptahydrate, although other magnesium sources in concentrations that contribute similar amounts of magnesium can be used. Typically, the concentration of magnesium in the culture medium is greater than about 0.5 g/L, preferably greater than about 1.0 g/L, and more preferably greater than about 2.0 g/L. Beyond certain concentrations, however, the addition of magnesium to the culture medium is not advantageous for the growth of the microorganisms. Accordingly, the concentration of magnesium in the culture medium is typically less than about 10 g/L, preferably less than about 5 g/L, and more preferably less than about 3 g/L. Further, in some instances, it may be desirable to allow the culture medium to become depleted of a magnesium source during culture.

In some embodiments, the culture medium can also include a biologically acceptable chelating agent, such as the dihydrate of trisodium citrate. In such instance, the concentration of a chelating agent in the culture medium is greater than about 0.2 g/L, preferably greater than about 0.5 g/L, and more preferably greater than about 1 g/L. Beyond certain concentrations, however, the addition of a chelating agent to the culture medium is not advantageous for the growth of the microorganisms. Accordingly, the concentration of a chelating agent in the culture medium is typically less than about 10 g/L, preferably less than about 5 g/L, and more preferably less than about 2 g/L.

The culture medium can also initially include a biologically acceptable acid or base to maintain the desired pH of the culture medium. Biologically acceptable acids include, but are not limited to, hydrochloric acid, sulfuric acid, nitric acid, phosphoric acid, and mixtures thereof. Biologically acceptable bases include, but are not limited to, ammonium hydroxide, sodium hydroxide, potassium hydroxide, and mixtures thereof. In some embodiments, the base used is ammonium hydroxide.

The culture medium can also include a biologically acceptable calcium source, including, but not limited to, calcium chloride. Typically, the concentration of the calcium source, such as calcium chloride, dihydrate, in the culture medium is within the range of from about 5 mg/L to about 2000 mg/L, preferably within the range of from about 20 mg/L to about 1000 mg/L, and more preferably in the range of from about 50 mg/L to about 500 mg/L.

The culture medium can also include sodium chloride. Typically, the concentration of sodium chloride in the culture medium is within the range of from about 0.1 g/L to about 5 g/L, preferably within the range of from about 1 g/L to about 4 g/L, and more preferably in the range of from about 2 g/L to about 4 g/L.

In some embodiments, the culture medium can also include trace metals. Such trace metals can be added to the culture medium as a stock solution that, for convenience, can be prepared separately from the rest of the culture medium. Typically, the amount of such a trace metals solution added to the culture medium is greater than about 1 mL/L, preferably greater than about 5 mL/L, and more preferably greater than about 10 mL/L. Beyond certain concentrations, however, the addition of a trace metals to the culture medium is not advantageous for the growth of the microorganisms. Accordingly, the amount of such a trace metals solution added to the culture medium is typically less than about 100 mL/L, preferably less than about 50 mL/L, and more preferably less than about 30 mL/L. It should be noted that, in addition to adding trace metals in a stock solution, the individual components can be added separately, each within ranges corresponding independently to the amounts of the components dictated by the above ranges of the trace metals solution.

The culture medium can include other vitamins, such as pantothenate, biotin, calcium, pantothenate, inositol, pyridoxine-HCl, and thiamine-HCl. Such vitamins can be added to the culture medium as a stock solution that, for convenience, can be prepared separately from the rest of the culture medium. Beyond certain concentrations, however, the addition of vitamins to the culture medium is not advantageous for the growth of the microorganisms.

The culture medium may be supplemented with hexanoic acid or hexanoate as a precursor for the cannabinoid biosynthetic pathway. The hexanoic acid may have a concentration of less than 3 mM hexanoic acid (e.g., from 1 nM to 2.9 mM hexanoic acid, from 10 nM to 2.9 mM hexanoic acid, from 100 nM to 2.9 mM hexanoic acid, or from 1 μM to 2.9 mM hexanoic acid) hexanoic acid.

The fermentation methods described herein can be performed in conventional culture modes, which include, but are not limited to, batch, fed-batch, cell recycle, continuous and semi-continuous. In some embodiments, the fermentation is carried out in fed-batch mode. In such a case, some of the components of the medium are depleted during culture, including pantothenate during the production stage of the fermentation. In some embodiments, the culture may be supplemented with relatively high concentrations of such components at the outset, for example, of the production stage, so that growth and/or production is supported for a period of time before additions are required. The preferred ranges of these components are maintained throughout the culture by making additions as levels are depleted by culture. Levels of components in the culture medium can be monitored by, for example, sampling the culture medium periodically and assaying for concentrations. Alternatively, once a standard culture procedure is developed, additions can be made at timed intervals corresponding to known levels at particular times throughout the culture. As will be recognized by those in the art, the rate of consumption of nutrient increases during culture as the cell density of the medium increases. Moreover, to avoid introduction of foreign microorganisms into the culture medium, addition is performed using aseptic addition methods, as are known in the art. In addition, a small amount of anti-foaming agent may be added during the culture.

The temperature of the culture medium can be any temperature suitable for growth of the genetically modified cells and/or production of compounds of interest. For example, prior to inoculation of the culture medium with an inoculum, the culture medium can be brought to and maintained at a temperature in the range of from about 20° C. to about 45° C., preferably to a temperature in the range of from about 25° C. to about 40° C. and more preferably in the range of from about 28° C. to about 32° C.

The pH of the culture medium can be controlled by the addition of acid or base to the culture medium. In such cases when ammonia is used to control pH, it also conveniently serves as a nitrogen source in the culture medium. Preferably, the pH is maintained from about 3.0 to about 8.0, more preferably from about 3.5 to about 7.0, and most preferably from about 4.0 to about 6.5.

In some embodiments, the carbon source concentration, such as the glucose concentration, of the culture medium is monitored during culture. Glucose or sucrose concentration of the culture medium can be monitored using known techniques, such as, for example, use of the glucose oxidase enzyme test or high pressure liquid chromatography, which can be used to monitor glucose concentration in the supernatant, e.g., a cell-free component of the culture medium. As stated previously, the carbon source concentration should be kept below the level at which cell growth inhibition occurs. Although such concentration may vary from organism to organism, for glucose as a carbon source, cell growth inhibition occurs at glucose concentrations greater than at about 60 g/L and can be determined readily by trial. Accordingly, when glucose is used as a carbon source the glucose is preferably fed to the fermentor and maintained below detection limits. Alternatively, the glucose concentration in the culture medium is maintained in the range of from about 1 g/L to about 100 g/L, more preferably in the range of from about 2 g/L to about 50 g/L, and yet more preferably in the range of from about 5 g/L to about 20 g/L. Although the carbon source concentration can be maintained within desired levels by addition of, for example, a substantially pure glucose solution, it is acceptable, and may be preferred, to maintain the carbon source concentration of the culture medium by addition of aliquots of the original culture medium. The use of aliquots of the original culture medium may be desirable because the concentrations of other nutrients in the medium (e.g. the nitrogen and phosphate sources) can be maintained simultaneously. Likewise, the trace metals concentrations can be maintained in the culture medium by addition of aliquots of the trace metals solution.

### EXAMPLES

The following examples are put forth to provide those of ordinary skill in the art with a description of how the compositions and methods described herein may be used, made, and evaluated, and are intended to be purely exemplary of the invention and are not intended to limit the scope of what the inventors regard as their invention.

### Example 1: Methods for Purifying and Decarboxylating Cannabinoids

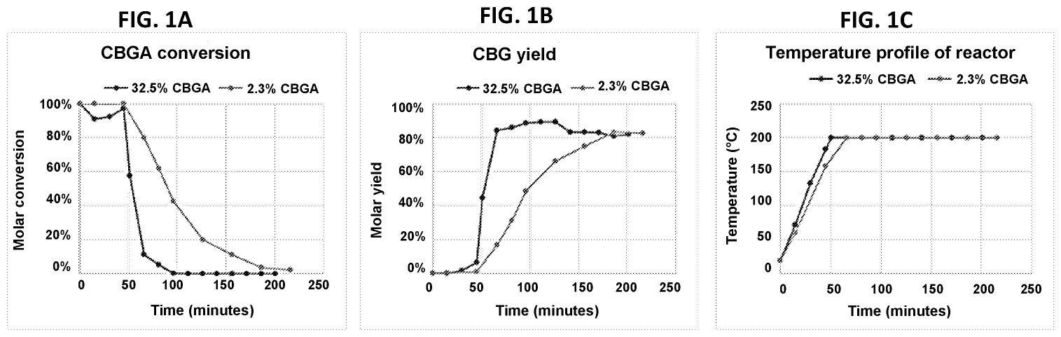

Decarboxylation is the reaction that converts acidic cannabinoids that are fermented or naturally occurring in plants to their neutral form. For example, decarboxylation converts cannabidiolic acid (CBDA) to cannabidiol (CBD). This process typically requires heat to drive the reaction. Reaction conditions for plant-derived cannabinoids have been reported to range from 100-180° C. for 0.5-10 hours (see U.S. Patent Application 2016/0214920, U.S. Pat. Nos. 9,376,367, 7,700,368, and 10,189,762). Prior to the use of a enzymatic composition including a serine protease, such as Tergazyme®, as a demulsification aid, decarboxylation of the fermented acidic cannabinoids in the oil overlay, specifically CBGA with initial concentrations ranging from 2-33 wt %, required 1-2 hours at 200° C. to achieve full conversion (see FIGS. 1A, 1B, and 1C). Even though a complete stoichiometric conversion to cannabigerol (CBG) is theoretically possible, molar yields of CBG>85% have not been demonstrated. The residence time of 1-2 hours at 200° C. for this reaction is further detrimental to the oil overlay, leading to thermal degradations that further complicates the purification process downstream. As product yield losses increase with the addition of purification steps, so does the overall cost of producing high purity cannabinoids by fermentation.

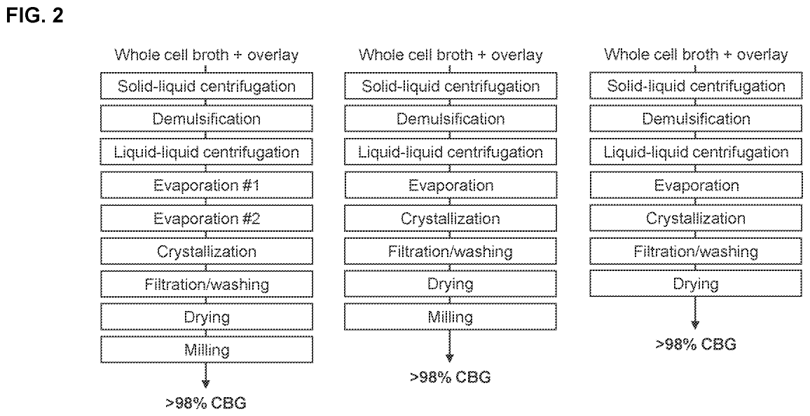

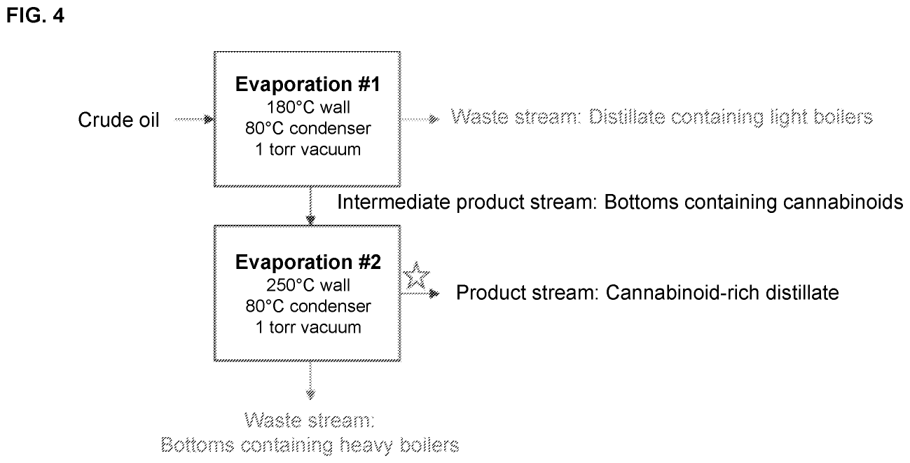

In the present method, the cannabinoid was purified by subjecting the whole cell broth and oil overlay to solid-liquid centrifugation, followed by a demulsification step using Tergazyme®, a liquid-liquid centrifugation step, evaporation using a short-path evaporator (e.g., a wiped-film evaporator), and a crystallization step (FIG. 2). The rapid decarboxylation of CBGA in the order of seconds (<1 minute) at a temperature of 180-250° C. was observed during the two evaporation steps carried out using a 2″ wiped-film evaporator of the oil overlay that was recovered from the fermentation composition, employing Tergazyme® as a demulsification aid (FIG. 4). A purity of 70 wt % CBG was demonstrated after 2 passes through the wiped-film evaporator, and, surprisingly, a complete stoichiometric conversion of CBGA to CBG, meaning 100% molar yield of CBG, was observed (see Method 3, Table 3). Additionally, negligible degradation of the vegetable oil was observed due to the low residence time at these high temperatures, further simplifying the purification process downstream.

TABLE 3
Decarboxylation conditions and distillate purity through
the iterations of the cannabinoid purification process.

| | Method 1 | Method 2 | Method 3 |
|---|---|---|---|
| Decarboxylation | 120-150 | 50-60 | <1 minute |
| time at 200° C. | minutes | minutes | |
| for complete | | | |
| CBGA conversion | | | |
| CBG yield from | 80-90% | 80-90% | 95-100% |
| decarboxylation | | | |
| Distillate purity | 30-35 wt % | 30-50 wt % | 70 wt % |
| | CBG | CBGA | CBG |
| | | 5-15 wt % | |
| | | CBG | |
| Overall product | 15-20% | 20-40% | 40-60% |
| recovery yield | demonstrated | estimated | estimated |

This process is especially advantageous for CBD purification; while CBG has been demonstrated to be thermally stable at a temperature of 200° C. for up to 3 hours, CBD has been shown to thermally degrade to tetrahydrocannabinol (THC) within 15 minutes at a temperature of 160-180° C. Aside from tetrahydrocannabinolic acid (THCA) production during fermentation, decarboxylation is expected to be the step with the highest risk of THC formation. The use of Tergazyme® upstream as a demulsification aid has significant processing advantageous; not only does it increase the overall product recovery yield; it further simplifies the purification process of fermentation-derived cannabinoids.

Complete conversion of CBGA is observed for a fermentation composition treated with Tergazyme® during the evaporation process, in comparison to the partial decarboxylation of ˜20% for the fermentation composition that was not treated with Tergazyme® as shown in Table 4. Treatment with Tergazyme® eliminates the need for a downstream decarboxylation step, and as such as such avoids further degradation of the residual vegetable oil overlay. As mentioned earlier, this processing method would be extremely useful as mitigation strategy for THC formation in the purification of CBD. Recovery yield through two evaporation passes was also improved from ˜70-75%, for fermentation compositions not treated with Tergazyme®, to ˜85-90%, for fermentation compositions treated with Tergazyme® (Table 5). Minimizing the number of process steps is critical to maintain an overall high recovery yield.

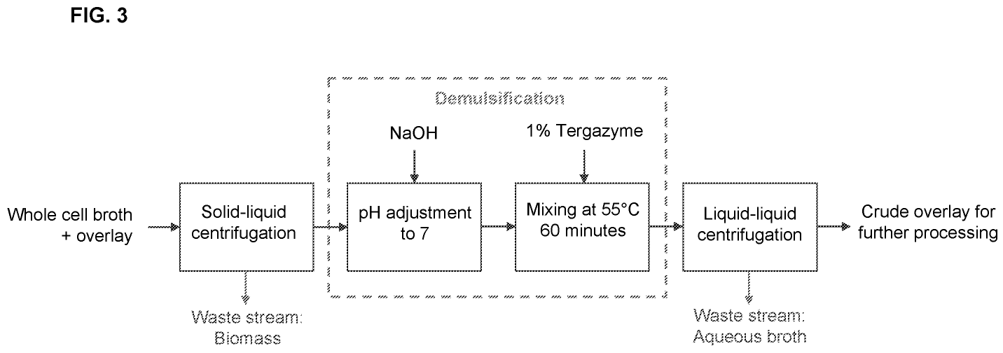

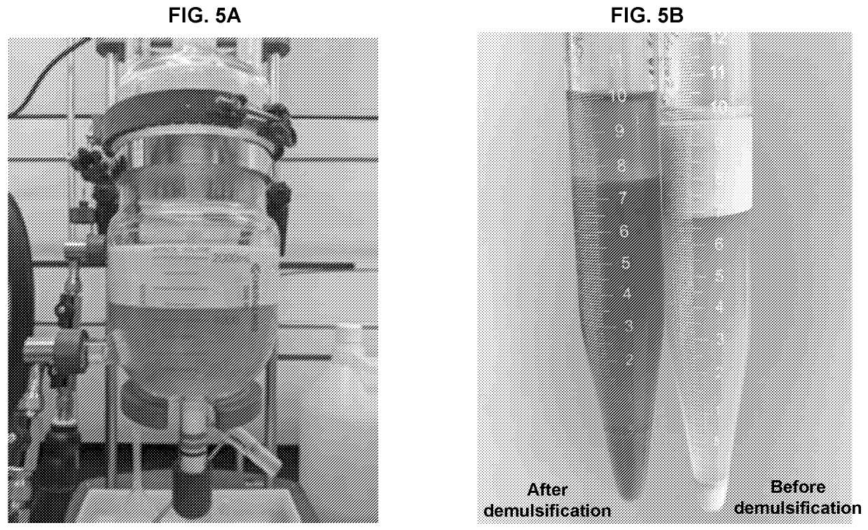

The objective of this work was to confirm that the 1% Tergazyme® treatment (see FIG. 3) led to the rapid decarboxylation of CBGA, either by catalyzing the reaction or removing a component (or components) from the overlay that was previously inhibiting the reaction. Overlay recovered without demulsification was reacted with 1% Tergazyme® at elevated temperatures, mimicking the demulsification process that was used to recover an overlay that was treated with 1% Tergazyme®. A stable emulsion layer was observed at the end of the reaction (see FIGS. 5A and 5B). ˜82% of the overlay was recovered by batch centrifugation, with the remaining lost to the emulsion layer. The change in color of the aqueous layer indicate that some products from this reaction is now water-soluble. Including the Tergazyme® treatment clearly had an impact on the feed material, and the process performance is similar to that of fermentation composition including Tergazyme® shown in Table 4. Complete conversion of CBGA was observed and a distillate stream with 65 wt % CBG was produced with high recovery yields (Table 5).

TABLE 4
Summary of evaporation data comparing overlay recovered with
and without Tergazyme ® as demulsification aid

| | | Without | With Tergazyme ®; Demulsification | | |
|---|---|---|---|---|---|
| | | Tergazyme ®; No | as shown in FIG. 3 | | |
| Key metrics | Calculation | demulsification | n = 1 | n = 2 | n = 3 |
| Mass balance | Total sample mass: | 99% | 97% | 97% | 97% |
| | OUT/IN | | | | |
| Molar balance | Mols of CBGA and | 100% | 98% | 108% * | 97% |
| | CBG: OUT/IN | | | | |
| CBGA | Mols of CBGA: | 19% | 98% | 100% | 100% |
| conversion | OUT/IN | | | | |
| CBG | Mols of CBG | 102% | 97% | 109% * | 97% |
| selectivity | generated/Moles of | | | | |
| | CBGA consumed | | | | |
| Yield by gain | Mols of | 72% | 85% | 97% * | 86% |
| | cannabinoids in the | | | | |
| | Final distillate/Feed | | | | |
| Yield by loss | 1-(Mols of | 72% | 87% | 89% | 89% |
| | cannabinoids in the | | | | |
| | Waste streams/Feed) | | | | |
| Distillate | — | 49.7 wt % CBGA | 70.2 wt | 70.1 wt | 70.9 wt |
| purity | | 13.3 wt % CBG | % CBG | % CBG | % CBG |

TABLE 5
Summary of evaporation data of the same feed, before
and after treatment with 1% Tergazyme ®

| | | Before treatment | |
|---|---|---|---|
| | | with Tergazyme ®; | After treatment |
| Key metrics | Calculation | No demulsification | with Tergazyme ® |
| Mass balance | Total sample mass: | 99% | 96% |
| | OUT/IN | | |
| Molar balance | Mols of CBGA and | 100% | 103% |
| | CBG: OUT/IN | | |
| CBGA | Mols of CBGA: | 19% | 99% |
| conversion | OUT/IN | | |
| CBG | Mols of CBG | 102% | 103% |
| selectivity | generated/Moles of | | |
| | CBGA consumed | | |
| Yield by gain | Mols of | 72% | 97% |
| | cannabinoids in the | | |
| | Final distillate/Feed | | |
| Yield by loss | 1-(Mols of | 72% | 95% |
| | cannabinoids in the | | |
| | Waste streams/Feed) | | |
| Distillate | — | 49.7 wt % CBGA | 65.1 wt % |
| purity | | 13.3 wt % CBG | |

The compositional data of the distillate stream generated during the second evaporation step (FIG. 4) for fermentation compositions with or without Tergazyme® treatment is summarized in Table 6. This data set was obtained from multiple assays spanning HPLC, GC-MS and GC-FID. While it is important to have an accurate titer measurement, it is equally important to know the identity and quantity of impurities in a process stream. The high levels of monoglycerides in the distillate from the fermentation composition treated with Tergazyme® indicate that there is potential room for optimization in the evaporation process, considering the boiling point for most monoglycerides is ˜100° C. higher than CBG.

TABLE 6
Composition data of distillate generated from with
or without treatment with Tergazyme ®

| | No Tergazyme ®; | With Tergazyme ®; |
|---|---|---|
| Component | No demulsification | Demulsification |
| CBGA | 49.7 | — |
| CBG | 13.3 | 70.4 |
| Free fatty acids | 3.2 | 3.6 |
| Monoglycerides | 7.4 | 18.3 |
| Diglycerides | 2.0 | 4.5 |
| Triglycerides | 4.0 | — |
| Olivetol | 0.08 | 1.7 |
| Olivetolic acid | 0.12 | — |
| PDAL | 1.6 | — |
| E,E-farnesol | 0.04 | — |
| Mass balance | 81.4 | 98.5 |
| Unknowns | 18.6 | 1.5 |

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

SEQUENCE APPENDIX

SEQ ID NO: 1 Subtilisin Carlsberg from Bacillus licheniformis (Tergazyme ®)
MMRKKSFWLGMLTAFMLVFTMAFSDSASAAQPAKNVEKDYIVGFKSGVKTASVKKDIIKESGGKVDK
QFRIINAAKAKLDKEALKEVKNDPDVAYVEEDHVAHALAQTVPYGIPLIKADKVQAQGFKGANVKVAVL
DTGIQASHPDLNVVGGASFVAGEAYNTDGNGHGTHVAGTVAALDNTTGVLGVAPSVSLYAVKVLNS
SGSGSYSGIVSGIEWATTNGMDVINMSLGGASGSTAMKQAVDNAYAKGVVVVAAAGNSGSSGNTNT
IGYPAKYDSVIAVGAVDSNSNRASFSSVGAELEVMAPGAGVYSTYPTNTYATLNGTSMASPHVAGAA
ALILSKHPNLSASQVRNRLSSTATYLGSSFYYGKGLINVEAAAQ
SEQ ID NO: 2-AAE candidate isolated from Pseudonocardia sp. N23
Amino acid sequence
MTAAQAPDPAGVPLVERTVPRMLARSAALDPDRPFVVTRERTWSHTDAHRIVATLAAAFTDRGIGQG
SRVAVMMPTSPRHVWLLLALAHLRAVPVALNPDASGEVLRYFVADSECVLGVVDQERAAAFATAAG
PDGPPAIVLPPGADDLGELGSAGPGPLDPGAASFSDTFVVLYTSGSTGMPKATAVTHAQVITCGAVF
TDRLGLGPADRLYTCLPLFHINATAYSLSGALVSGASLALGPHFSATTFWDDVADLGATEVNAMGSM
VRILQSRPPRPAERAHRVRTMFVAPLPPDAVELSERFGLDFATCYAQTEWLPSSMTRPGEGYGRPG
ATGPVLPWTEVRIVGDDDRPLPAGQTGEIILRPRDPYTTFQGYLGKPQETVDAWRNLWFHTGDLGDI
GPDGWLHYRGRRKDVIRRRGENIPATVVEDLLAGHPDIAEVAAVSVPAHISEEEIFAFVVPGAGAALT
TADVEAHAHAVLPRYMVPSYLALVPDLPRTATNKIAKVELTERARAAVEGTGDPADAPTRTSAADRV
VVPAAE
SEQ ID NO: 3-AAE candidate isolated from Pseudomonas putida
Amino acid sequence
MMVPTLEHELAPNEANHVPLSPLSFLKRAAQVYPQRDAVIYGARRYSYRQLHERSRALASALERVGV
QPGERVAILAPNIPEMLEAHYGVPGAGAVLVCINIRLEGRSIAFILRHCAAKVLICDREFGAVANQALAM
LDAPPLLVGIDDDQAERADLAHDLDYEAFLAQGDPARPLSAPQNEWQSIAINYTSGTTGDPKGVVLH
HRGAYLNACAGALIFQLGPRSVYLWTLPMFHCNGWSHTWAVTLSGGTHVCLRKVQPDAINAAIAEHA
VTHLSAAPVVMSMLIHAEHASAPPVPVSVITGGAAPPSAVIAAMEARGFNITHAYGMTESYGPSTLCL
WQPGVDELPLEARAQFMSRQGVAHPLLEEATVLDTDTGRPVPADGLTLGELVVRGNTVMKGYLHNP
EATRAALANGWLHTGDLAVLHLDGYVEIKDRAKDIIISGGENISSLEIEEVLYQHPEVVEAAVVARPDS
RWGETPHAFVTLRADALASGDDLVRWCRERLAHFKAPRHVSLVDLPKTATGKIQKFVLREWARQQE
AQIADAEH
SEQ ID NO: 4-AAE candidate isolated from Streptomyces sp. ADI96-02
Amino acid sequence
MLSTMQDVPLTVTRILQHGMTIHGKSQVTTWTGEPEPHRRTFAEIGARATRLAHALRDELGIDGDQR
VATLMWNNAEHVEAYLAVPSMGAVLHTLNLRLPAEQLIWIVNHADDKVVIVNGSLLPLLVPLLPHLPTV
EHVVVSGPGDRSALAGVAPRVHEYEELIADRPTTYDWPELDERQAAAMCYTSGTTGDPKGVVYSHR
SVYLHSMQVNMTESMGLTDKDTTLVVVPQFHVNAWGLPHATFMAGVNMLMPDRFLQPAPLADMIE
RERPTHAAAVPTIWQGLLAEVTAHPRDLTSMASVTIGGAACPPSLMEAYDKLGVRLCHAWGMTETS
PLGTMANPPAGLSAEEEWPYRVTQGRFPAGVEARLVGPAGDHLPWDGRSAGELEVRGAWIAGAYY
GGADGEHLRPEDKFSADGWLKTGDVGVISADGFLTLTDRAKDVIKSGGEWISSVELENALMAHPDVA
EAAVVAVPDEKWGERPLATVVLKEGAEVGYEALKVFLADSGIAKWQLPERWTVIPAVPKTSVGKFDK
KVIRKQYADGELDITQL
SEQ ID NO: 5-AAE candidate isolated from Erythrobacter citreus LAMA 915
Amino acid sequence
MSRAECRDRLTAPGERFEIETIDIRGVPTRVWKHAPTNMRQVAMAARTHGDRLFAIYEDERVTYEAW
FRAVARMAAELRERGVAKGDRVALAMRNLPEWPVAFFAATTIGAICVPLNAWWTGPELAFGLANSG
AKLLVCDAERWERIAPHRGELPDLEHALVSRSDAPLEGAEQLEDLLGTPKDYAALPSAALPQVDIDPE
DEATIFYTSGTTGQPKGALGTHRNLCTNIMSSAYNGAIAFLRRGEEPPAPVQKVGLTVIPLFHVTACSA
GLMGYVVAGHTMVFMHKWDPVKAFQLIEREKVNLTGGVPTIAWQLLEHPERANYDLSSLEAVAYGG
APAAPELVRKIHEEFGALPANGWGMTETMATVTGHSSEDYLNRPDSCGPPVAVADLKIVGDDGVTEL
PVGEVGELWARGPMVVKGYWNRPEATAETFVDGWVRTGDLARLDEEGWCYIVDRAKDMIIRGGENI
YSSEVENVLYDHPAVTDAALVAIAHPTLGEEPAAVVHLAPGMSATEDELREWVAARLAKFKVPVRIAF
VQDTLPRNANGKILKKDLGAFFA
SEQ ID NO: 6-AAE candidate isolated from Saccharomyces cerevisiae
Amino acid sequence
MVAQYTVPVGKAANEHETAPRRNYQCREKPLVRPPNTKCSTVYEFVLECFQKNKNSNAMGWRDVK
EIHEESKSVMKKVDGKETSVEKKWMYYELSHYHYNSFDQLTDIMHEIGRGLVKIGLKPNDDDKLHLYA
ATSHKWMKMFLGAQSQGIPVVTAYDTLGEKGLIHSLVQTGSKAIFTDNSLLPSLIKPVQAAQDVKYIIH
FDSISSEDRRQSGKIYQSAHDAINRIKEVRPDIKTFSFDDILKLGKESCNEIDVHPPGKDDLCCIMYTSG
STGEPKGVVLKHSNVVAGVGGASLNVLKFVGNTDRVICFLPLAHIFELVFELLSFYWGACIGYATVKTL
TSSSVRNCQGDLQEFKPTIMVGVAAVWETVRKGILNQIDNLPFLTKKIFWTAYNTKLNMQRLHIPGGG
ALGNLVFKKIRTATGGQLRYLLNGGSPISRDAQEFITNLICPMLIGYGLTETCASTTILDPANFELGVAG
DLTGCVTVKLVDVEELGYFAKNNQGEVWITGANVTPEYYKNEEETSQALTSDGWFKTGDIGEWEAN
GHLKIIDRKKNLVKTMNGEYIALEKLESVYRSNEYVANICVYADQSKTKPVGIIVPNHAPLTKLAKKLGI
MEQKDSSINIENYLEDAKLIKAVYSDLLKTGKDQGLVGIELLAGIVFFDGEWTPQNGFVTSAQKLKRKD
ILNAVKDKVDAVYSSS
SEQ ID NO: 7-AAE candidate isolated from Citreicella sp. SE45
Amino acid sequence
MSLSTEETARRRTLAEGAGYDALREGFRWPGAARVNMAEQVCDSWAAREPGRPAILDMRAGGAPE
VVSYGALQALSRRVEAWFRGQGVARGDRVGVLLSQSPLCAAAHIAAWRMGAISVPLFKLFKHDALE
SRLGDSGARVVVSDDEGAAMLAPFGLSVVTEAGLPQDGATEPAADTGPEDPAIIIYTSGTTGKPKGAL
HGHRVLTGHLPGVEMSHDLLGQPGDVLWTPADWAWIGGLFDVLMPGLYLGVPVVAARMPRFEISEC
LRICQQASVRNVFFPPTAFRMLKSEGAELPGLRSVASGGEPLGAEMLAWGRKAFGVEINEFYGQTE
CNMVASSCGALFRARPGCIGKPAPGFHIAVIDEDGNETDGEGDVAIRRGAGSMLLEYWQKPQETAD
KFRGDWLVTGDRGTWEDGYLRFVGREDDVITSAGYRIGPTEIEDCLMTHPAVATVGVVGKPCPLRTE
LVKAYVVLRPGVEVRASELQAWVKERLATYSYPREIAFLDALPMTVTGKVIRKELKAIAAAERTAEAAG
EVS
SEQ ID NO: 8-AAE candidate isolated from Bacillus subtilis (strain 168)
Amino acid sequence
MNLVSKLEETASEKPDSIACRFKDHMMTYQELNEYIQRFADGLQEAGMEKGDHLALLLGNSPDFIIAF
FGALKAGIVVVPINPLYTPTEIGYMLTNGDVKAIVGVSQLLPLYESMHESLPKVELVILCQTGEAEPEAA
DPEVRMKMTTFAKILRPTSAAKQNQEPVPDDTAVILYTSGTTGKPKGAMLTHQNLYSNANDVAGYLG
MDERDNVVCALPMFHVFCLTVCMNAPLMSGATVLIEPQFSPASVFKLVKQQQATIFAGVPTMYNYLF
QHENGKKDDFSSIRLCISGGASMPVALLTAFEEKFGVTILEGYGLSEASPVTCFNPFDRGRKPGSIGT
SILHVENKVVDPLGRELPAHQVGELIVKGPNVMKGYYKMPMETEHALKDGWLYTGDLARRDEDGYF
YIVDRKKDMIIVGGYNVYPREVEEVLYSHPDVKEAVVIGVPDPQSGEAVKGYVVPKRSGVTEEDIMQH
CEKHLAKYKRPAAITFLDDIPKNATGKMLRRALRDILPQ
SEQ ID NO: 9-AAE candidate isolated from Saccharomyces cerevisiae
Amino acid sequence
MTEQYSVAVGEAANEHETAPRRNIRVKDQPLIRPINSSASTLYEFALECFTKGGKRDGMAWRDIIDIH
ETKKTIVKRVDGKDKPIEKTWLYYELTPYITMTYEEMICVMHDIGRGLIKIGVKPNGENKFHIFASTSHK
WMKTFLGCMSQGIPVVTAYDTLGESGLIHSMVETDSVAIFTDNQLLSKLAVPLKTAKNVKFVIHNEPID
PSDKRQNGKLYKAAKDAVDKIKEVRPDIKIYSFDEIIEIGKKAKDEVELHFPKPEDPACIMYTSGSTGTP
KGVVLTHYNIVAGIGGVGHNVIGWIGPTDRIIAFLPLAHIFELTFEFEAFYWNGILGYANVKTLTPTSTRN
CQGDLMEFKPTVMVGVAAVWETVRKGILAKINELPGWSQTLFWTVYALKERNIPCSGLLSGLIFKRIR
EATGGNLRFILNGGSAISIDAQKFLSNLLCPMLIGYGLTEGVANACVLEPEHFDYGIAGDLVGTITAKLV
DVEDLGYFAKNNQGELLFKGAPICSEYYKNPEETAAAFTDDGWFRTGDIAEWTPKGQVKIIDRKKNLV
KTLNGEYIALEKLESIYRSNPYVQNICVYADENKVKPVGIVVPNLGHLSKLAIELGIMVPGEDVESYIHE
KKLQDAVCKDMLSTAKSQGLNGIELLCGIVFFEEEWTPENGLVTSAQKLKRRDILAAVKPDVERVYKE
NT
SEQ ID NO: 10-AAE candidate isolated from Bhargavaea cecembensis DSE10
Amino acid sequence
MYTDHGWIMKRADITPDGTALIDVHTGQRWTYRELAGRTAAYMEQFRSAGLRKGERVAVLSHNRIDL
FAVLFACAGRGLIYVPMNWRLSESELRYIVSDSGPSLLLHDHEHAGRAAGLGIPAALLDSVPATSVNL
RTEQAAGRLDDPWMMIYTGGTTGRPKGVVLTFESVNWNAINTIISWNLSARDCTLNYMPLFHTGGLN
ALSLPILMAGGTVVIGRKFDPEEAIRALNDYRTTISLFVPTMHQAMLDTDLFWESDFPTVDVFLSGGAP
CPQTVYDAYRKKGVRFREGYGMTEAGPNNFIIDPDTAMRKRGAVGKSMQFNEVRILDAKGRPCRAG
EVGELHLRGRHLFSHYWNNEEATQEALKEGWFSTGDLASRDEDGDYFIVGRKKEMIISGGENIYPQE
VEQCLIGHDGVREIAVIGIADRKWGERVVAFIVAQPGNIPKTEELLKHCAQTLGSYKVPKDFFFVQELPI
TDIGKIDKKQLAIMAEELKKEEMQHPGQSG
SEQ ID NO: 11-AAE candidate isolated from Deltaproteobacteria bacterium ADurb.Bin022
Amino acid sequence
MHKFTLDKPDNLVDWWGESVTRFADRPLFGTKNKEGVYKWATYKEIGNRIDNLRAGLTQLGIGKDD
VVGIIANNRPEWAVIGFATWGCLARYVPMYEAELVQVWKYIINDSGAKVLFVSNPAIYEKIKDFPKDIPT
LKHIFIIESDGDNSMASLEKKGAAKPVAPKSPKAEDVAELIYTSGTTGNPKGVLLMHMNFTSNSHAGL
KMYPELYENEVVSLTILPWAHVFGQTAELFAIIRLGGRMGLIESTKTIINDIVQIKPTFIIAVPTVFNRIYDG
LWNKMNKDGGLARALFVMGVEAAKKKRILAEKGQSDLMTNFKVAVADKIVFKKIRERMGGRMLGSM
TGSAAMNVEISKFFFDIGIPIYDCYGLTETSPGITMNGSQAYRIGSVGRPIDKVKVVIDSSVVEEGATDG
EIIAYGPNVMKGYHNRPEDTKAALTPDGGFRTGDRGRLDKDGYLFITGRIKEQYKLENGKFCFPVSLE
ENICLASFVQQAVVYGLNRPYNVCIVVPDFDVLLDYAKEKGLPTDIKTLVEREDIIHMISEAVTGQLKGK
FGGYEIPKKFIILPEAFSLDNGMLTQTMKLKRKVILDKLNDRIEALYKEDK
SEQ ID NO: 12-AAE candidate isolated from Alcaligenes xylosoxydans (Achromobacter
xylosoxidans)
Amino acid sequence
MYSRIHEPHACTLTDALREWAASRPAAPWLEDSQGIAFTVGQAFTSSQRFASFLHHQLGVQPEERV
GVFMSNSCAMVATTFGIGYLRATAVMLNTELRSSFLRHQLNDCQLATIVVDSALVEHVASLADELPHL
RTLVVVGDAPAAVPERWRQVAWMDSSACAPWEGPAPRPEDIFCIMYTSGTTGPSKGVLMPHCHCA
LLGLGAIRSLEITEADKYYICLPLFHANGLFMQLGATVLAGIPAFLKQRFSASTWLADIRRSGATLTNHL
GTTAMFVINQPPTEQDRDHRLRASLSAPNPAQHEAVFRERFGVKDVLSGFGMTEVGIPIWGRIGHAA
PNAAGWAHEDRFEICIADPETDVPVLAGQVGEILVRPKVPFGFMAGYLNVPAKTVEAWRNLWFHTG
DAGTRDEQGLITFVDRIKDCIRRRGENISATEVEVVVGQLPGVHEVAAYAVPAQGAGGEDEVMLALV
PSEGAALDMADIVRQASAQLPRFAKPRYLRQMDSLPKTATGKIQRAVLRQQGSAGAYDAEAAPAR
SEQ ID NO: 13-AAE candidate isolated from Novosphingobium sp. MD-1
Amino acid sequence
MQFTQGLERAVQHHPDVTATICRARSQTFAELYERVTGLAGCLASRSLAKGARIAVLALNSDHYLEVY
LATAWAGGVIVPVNFRWSPAEIAYSLNDAGCVALMVDQHHAALVPTLREQCPGLQHIFLMGGTEESD
DLPGLDALIAAAEPLQNAGAGGDDLLGIFYTGGTTGRPKGVMLSHANLCSSGLSMLAEGVFNEGAVG
LHVAPMFHLADMLLTTCLVLRGCTHVMLPAFSPDAVLDHVARFGVTDTLVVPAMLQAIVDHPAIGNFD
TSSLCNILYGASPASETLLRRTMAAFPDVRLTQGYGMTESAAFICALPWHQHVVDNDGPNRLRAAGR
STFDVHLQIVDPDDRELPRGEIGEIIVKGPNVMQGYYNMPEATAETLRGGWLHTGDMAWMDEEGYV
FIVDRAKDMIISGGENIYSAEVENAVASHPAVAANAVIGIPHEQMGEAVHVALVLRPGSELSLEALQAH
CRALIAGYKVPRSMEVRPSLPLSGAGKILKTELREPFWKGRDRAVG
SEQ ID NO: 14-AAE candidate isolated from Arabidopsis thaliana (Mouse-ear cress)
Amino acid sequence
MEDSGVNPMDSPSKGSDFGVYGIIGGGIVALLVPVLLSVVLNGTKKGKKRGVPIKVGGEEGYTMRHA
RAPELVDVPWEGAATMPALFEQSCKKYSKDRLLGTREFIDKEFITASDGRKFEKLHLGEYKWQSYGE
VFERVCNFASGLVNVGHNVDDRVAIFSDTRAEWFIAFQGCFRQSITVVTIYASLGEEALIYSLNETRVS
TLICDSKQLKKLSAIQSSLKTVKNIIYIEEDGVDVASSDVNSMGDITVSSISEVEKLGQKNAVQPILPSKN
GVAVIMFTSGSTGLPKGVMITHGNLVATAAGVMKVVPKLDKNDTYIAYLPLAHVFELEAEIVVFTSGSA
IGYGSAMTLTDTSNKVKKGTKGDVSALKPTIMTAVPAILDRVREGVLKKVEEKGGMAKTLFDFAYKRR
LAAVDGSWFGAWGLEKMLWDALVFKKIRAVLGGHIRFMLVGGAPLSPDSQRFINICMGSPIGQGYGL
TETCAGATFSEWDDPAVGRVGPPLPCGYVKLVSWEEGGYRISDKPMPRGEIVVGGNSVTAGYFNN
QEKTDEVYKVDEKGTRWFYTGDIGRFHPDGCLEVIDRKKDIVKLQHGEYVSLGKVEAALGSSNYVDN
IMVHADPINSYCVALVVPSRGALEKWAEEAGVKHSEFAELCEKGEAVKEVQQSLTKAGKAAKLEKFE
LPAKIKLLSEPWTPESGLVTAALKIKREQIKSKFKDELSKLYA
SEQ ID NO: 15-AAE candidate isolated from Bradyrhizobium sp. CI-41S
Amino acid sequence
MDWSQHAIPPMRLEPRFGDRVVPAFVDRPASLWAMIADAVAQNGGGEALVCGDIRISWHEVARRAA
KVAAGFAKLGLNSGDRVAILLGNRIEFVLTMFAAAHAGLVTVLLSTRQQKPEIAYVLNDCGARALVHEA
TLAERIPDAADIPGLAHRIAVSDDAASQFAVLLDHPPAPAPAAVSEEDTAMILYTSGTTGRPKGAMLAH
CNIIHSSMVFASTLRLTQADRSIAAVPLAHVTGAVANITTMVRCAGTLIIMPEFKAAEYLKVAARERVSY
TVMVPAMYNLCLLQPDFDSYDLSSWRIGGFGGAPMPVATIERLDAKIPGLKLANCYGATETTSPSTLM
PGELTAAHIDSVGLPCPGAEIIVMGPDGRELPRGEIGELWIRSASVIKGYWNNPKATAESFTDGFWHS
GDLGSVDAENFVRVFDRQKDMINRGGLKIYSAEVESVLAGHPAVIESAIIAKPCPVLGERVHAVIVTRT
EVDAESLRAWCAERLSDYKVPETMTLTTTPLPRNANGKVVKRQLRETLAAGQAPA
SEQ ID NO: 16-AAE candidate isolated from Bradyrhizobium sp. CI-41S
Amino acid sequence
MAGPAVLTVADTIARSFLLAVQTRGDRPAIREKKFGIWQPTSWREWLQISKDIAHGLHASGFRPGDVA
SIIANAVPEWVYADMGILCAGGVSSGIYPTDSTAQVEYLVNDSRTKIVFVEDEEQLDKVLACRARCPTL
EKIVVFDMEGLSGFSDPMVLSFAEFAALGRNHAHGNAALWDEMTGSRTASDLAILVYTSGTTGPPKG
AMHSNRSVTHQMRHANDLFPSTDSEERLVFLPLCHVAERVGGYYISIALGSVMNFAESPETVPDNLR
EVQPTAFLAVPRVWEKFYSGITIALKDATPFQNWMYGRALAIGNRMTECRLEGETPPLSLRLANRAAY
WLVFRNIRRMLGLDRCRIALTGAAPISPDLIRWYLALGLDMREVYGQTENCGVATIMPTERIKLGSVG
KAAPWGEVMICPKGEILIKGDFLFMGYLNQPERTAETIDAKGWLHTGDVGTIDNEGYVRITDRMKDIIIT
SGGKNVTPSEIENQLKFSPYVSDAVVIGDKRPYLTCLIMIDQENVEKFAQDHDIPFTNYASLCRAREIQ
DLIQREVEAVNTKFARVETIKKFYLIERQLTPEDEELTPTMKLKRSFVNKRYAAEIDAMYGARAVA
SEQ ID NO: 17-AAE candidate isolated from Thermus thermophilus (strain HB8/
ATCC 27634/DSM 579)
Amino acid sequence
MEGERMNAFPSTMMDEELNLWDFLERAAALFGRKEVVSRLHTGEVHRTTYAEVYQRARRLMGGLR
ALGVGVGDRVATLGFNHFRHLEAYFAVPGMGAVLHTANPRLSPKEIAYILNHAEDKVLLFDPNLLPLV
EAIRGELKTVQHFVVMDEKAPEGYLAYEEALGEEADPVRVPERAACGMAYTTGTTGLPKGVVYSHR
ALVLHSLAASLVDGTALSEKDVVLPVVPMFHVNAWCLPYAATLVGAKQVLPGPRLDPASLVELFDGE
GVTFTAGVPTVWLALADYLESTGHRLKTLRRLVVGGSAAPRSLIARFERMGVEVRQGYGLTETSPVV
VQNFVKSHLESLSEEEKLTLKAKTGLPIPLVRLRVADEEGRPVPKDGKALGEVQLKGPWITGGYYGN
EEATRSALTPDGFFRTGDIAVWDEEGYVEIKDRLKDLIKSGGEWISSVDLENALMGHPKVKEAAVVAI
PHPKWQERPLAVVVPRGEKPTPEELNEHLLKAGFAKWQLPDAYVFAEEIPRTSAGKFLKRALREQYK
NYYGGA
SEQ ID NO: 18-AAE candidate isolated from Microbacterium oxydans
Amino acid sequence
MVRSTYPDVEIPEVSIHDFLFGDLSEAELDTVALVDGMSGATTTYRQLVGQIDLFAGALAARGVGVGT
TVGVLCPNVPAFATVFHGILRAGATATTINSLYTADEIANQLTDAGATWLVTVSPLLPGAQAAAEKLGF
DADHVIVLDGAEGHPSLPALLGEGRQAPDVSFDPSTHLAVLPYSSGTTGRPKGVMLTHRNLVANVSQ
CQPVLGVDASDRVLAVLPFFHIYGMTVLLNFALRQRAGLATMPRFDLPEFLRIIAEHRTSWVFVAPPIA
VALAKHPIVDQYDLSAVKVIFSGAAPLDGTLASAVANRLGCIVTQGYGMTETSPAVNLISEARTEIDRS
TIGPLVPNTEARLVDPDSGEDVVVPAEGASEPGELWVRGPQVMVGYLNRPDATAEMLDADGWLHT
GDVATVTHDGIYRIVDRLKELIKYKGYQVAPAVLEAVLLEHPAIADAAVIGAFDDDGQEVPKAFVVRQP
DADLDADAVMAHVTSHVAPHEKVRQVEFIDVIPKSSSGKILRKDLRAR
SEQ ID NO: 19-AAE candidate isolated from Arabidopsis thaliana (Mouse-ear cress)
Amino acid sequence
MSLAADNVLLVEEGRPATAEHPSAGPVYRCKYAKDGLLDLPTDIDSPWQFFSEAVKKYPNEQMLGQ
RVTTDSKVGPYTWITYKEAHDAAIRIGSAIRSRGVDPGHCCGIYGANCPEWIIAMEACMSQGITYVPLY
DSLGVNAVEFIINHAEVSLVFVQEKTVSSILSCQKGCSSNLKTIVSFGEVSSTQKEEAKNQCVSLFSWN
EFSLMGNLDEANLPRKRKTDICTIMYTSGTTGEPKGVILNNAAISVQVLSIDKMLEVTDRSCDTSDVFF
SYLPLAHCYDQVMEIYFLSRGSSVGYWRGDIRYLMDDVQALKPTVFCGVPRVYDKLYAGIMQKISAS
GLIRKKLFDFAYNYKLGNMRKGFSQEEASPRLDRLMFDKIKEALGGRAHMLLSGAAPLPRHVEEFLRII
PASNLSQGYGLTESCGGSFTTLAGVFSMVGTVGVPMPTVEARLVSVPEMGYDAFSADVPRGEICLR
GNSMFSGYHKRQDLTDQVLIDGWFHTGDIGEWQEDGSMKIIDRKKNIFKLSQGEYVAVENLENTYSR
CPLIAQIWVYGNSFESFLVGVVVPDRKAIEDWAKLNYQSPNDFESLCQNLKAQKYFLDELNSTAKQY
QLKGFEMLKAIHLEPNPFDIERDLITPTFKLKRPQLLQHYKGIVDQLYSEAKRSMA
SEQ ID NO: 20-AAE candidate isolated from Brevibacterium yomogidense
Amino acid sequence
MSWFDERPWLRTLGLTETEAVPLEPSTPLRDLADTVAAHPTTAAWTHYGQSATYAEFDRQTTAFAA
YLAESGIRPGDAVAVYAQNSPHFPIATYGIWKAGAVVVPLNPMYRDELTHAFADADVKAIVVQKALYL
MRVKEYAADLPLVVLAGDLDWAQDGPDAVFGAYADLPDVPLPDLRTVVDERLDTDFEPLTVRPEDP
ALIGYTSGTSGKAKGALHPHSSISSNSRMAARNAGLPQGAGVVSLAPLFHITGFICQMIASTANGSTLV
LNHRFDPASFLDLLRQEKPAFMAGPATVYTAMMASPSFGADAFDSFHSIMSGGAPLPEGLVKRFEEK
TGHYIGQGYGLTETAAQAVTVPHSLRAPVDPESGNLSTGLPQRDAMVRILDDDGNPVGPREVGEVAI
SGPMVATEYLGNPQATADSLPGGELRTGDVGFMDPDGWVFIVDRKKDMINASGFKVWPREVEDILY
MHPAVREGAVVGVPDEYRGETVVAFVSLQPDSQATAEDIIAHCKEHLASYKAPVEVTIVDELPKTSSG
KILRRTVRDEATQARQAQPDAH
SEQ ID NO: 21-AAE candidate isolated from Nocardioides simplex (Arthrobacter simplex)
Amino acid sequence
MSFRYYRDLHPTFADRTEWALPTVLRHHAAERPDAVWLDCPEEGRTWTFAETLTAAERVGRSLLAA
GAEPGDRVVLVAQNSSAFVRTWLGTAVAGLVEVPVNTAYEHDFLAHQVSTVEATLAVVDDVYAARF
VAIAEAAKSIRKFWVIDTGSRDQALATLRDAGWEAAPFEELDEAATAPEVVDATLALPDVRPQDLASV
LFTSGTTGPSKGVAMPHAQMYFFADECVSLVRLTPDDAWMSVTPLFHGNAQFMAAYPTLVAGARFV
TRSRFSASRWVDQLRESRVTVTNFIGVMMDFIWKQDRRDDDADNPLRVVFAAPTAATLVGPMSERY
GIEAFVEVFGLTETSAPIISPYGVDRPAGAAGLAADEWFDVRLVDPETDEEVGVGEIGELVVRPKVPFI
CSMGYFNMPDKTVEAWRNLWFHTGDALRRDEDGWFYFVDRFKDALRRRGENISSYEIETSILAHPA
VVECAVIAVPASSEAGEDEVMAYVITGGDAPVPTPAELWAHCDGRIPSFAVPRYLRFVDEMPKTPSQ
RVQKAKLRALGVTPDTHDREA
SEQ ID NO: 22-AAE candidate isolated from Brevibacterium linens
Amino acid sequence
MTVTEEFRAARDKLIELRSDYDAAREQFEWPRFDHFNFALDWFDKIAENNDKPALWIVEQDGSEGK
WSFAELSARSNQVANHFRRAGIKRGDHVMVMLNNQVELWETMLAGIKLGAVLMPATTQLGPIDLTD
RAERGHAEFVVAGAEDAAKFDDVDVEVVRIVVGGEPTRQQDYSYSDADDESTEFDPQGSSRADDL
MLLYFTSGTTSKAKMVAHTHVSYPVGHLSTMYWMGLTPGDVHLNVASPGWAKHAWSNIFTPWIAEA
CVFLYNYSRFDANALMETMDRVGVTSFCAPPTVWRMLIQADLKHLKTPPTKALGAGEPLNPEIIDRVH
SDWGVLIRDGFGQTESTLQIGNSPDQELKYGSMGKALPGFDVVLIDPATGEEGDEGEICLRLDPRPIG
LTTGYWSNPEKTAEAFEGGVYHTGDVASRDEDGFITYVGRADDVFKASDYRLSPFELESVLIEHEAV
AEAAVVPSPDPVRLAVPKAYVVVSSKFDADAETARSILAYCREHLAPYKRIRRLEFAELPKTISGKIRR
VELRAREDQLHPFSGEPVVEGNEYADTDFDLKS
SEQ ID NO: 23-AAE candidate isolated from Pseudomonas putida (Arthrobacter
siderocapsulatus)
Amino acid sequence
MNLGKIITRSARYWPDHTAVADSQTRLTYAQLERRSNRLASGLGALGVATGEHVAILAANRVELVEAE
VALYKAAMVKVPINARLSLDEVVRVLEDSCSVALITDATFAQALAERRAALPMLRQVIALEGEGGDLG
YAALLERGSEAPCSLDPADDALAVLHYTSGSSGVLKAAMLSFGNRKALVRKSIASPTRRSGPDDVMA
HVGPITHASGMQIMPLLAVGACNLLLDRYDDRLLLEAIERERVTRLFLVPAMINRLVNYPDVERFDLSS
LKLVMYGAAPMAPALVKKAIELFGPILVQGYGAGETCSLVTVLTEQDHLIEDGNYQRLASCGRCYFET
DLRVVNEAFEDVAPGEIGEIVVKGPDIMQGYWRAPALTAEVMRDGYYLTGDLATVDAQGYVFIVDRK
KEMIISGGFNVYPSEVEQVIYGFPEVFEAAVVGVPDEQWGEAVRAVVVLKPGAQLDAAELIERCGRAL
AGFKKPRGVDFVTELPKNPNGKVVRRLVREAYWQHSDRRI
SEQ ID NO: 24-AAE candidate isolated from Drosophila melanogaster (Fruit fly)
Amino acid sequence
MNDLKPATSYRSTSLHDAVKLRLDEPSSFSQTVPPQTIPEFFKESCEKYSDLPALVWETPGSGNDGW
TTLTFGEYQERVEQAALMLLSVGVEERSSVGILAFNCPEWFFAEFGALRAGAVVAGVYPSNSAEAVH
HVLATGESSVCVVDDAQQMAKLRAIKERLPRLKAVIQLHGPFEAFVDHEPGYFSWQKLQEQTFSSEL
KEELLARESRIRANECAMLIFTSGTVGMPKAVMLSHDNLVFDTKSAAAHMQDIQVGKESFVSYLPLSH
VAAQIFDVFLGLSHAGCVTFADKDALKGTLIKTFRKARPTKMFGVPRVFEKLQERLVAAEAKARPYSR
LLLARARAAVAEHQTTLMAGKSPSIYGNAKYWLACRVVKPIREMIGVDNCRVFFTGGAPTSEELKQFF
LGLDIALGECYGMSETSGAITLNVDISNLYSAGQACEGVTLKIHEPDCNGQGEILMRGRLVFMGYLGL
PDKTEETVKEDGWLHSGDLGYIDPKGNLIISGRLKELIITAGGENIPPVHIEELIKKELPCVSNVLLIGDH
RKYLTVLLSLKTKCDAKTGIPLDALREETIEWLRDLDIHETRLSELLNIPADLQLPNDTAALAATLEITAK
PKLLEAIEEGIKRANKYAISNAQKVQKFALIAHEFSVATGELGPTLKIRRNIVHAKYAKVIERLYK
SEQ ID NO: 25-AAE candidate isolated from Cannabis sativa
Amino acid sequence
MGKNYKSLDSVVASDFIALGITSEVAETLHGRLAEIVCNYGAATPQTWINIANHILSPDLPFSLHQMLFY
GCYKDFGPAPPAWIPDPEKVKSTNLGALLEKRGKEFLGVKYKDPISSFSHFQEFSVRNPEVYWRTVL
MDEMKISFSKDPECILRRDDINNPGGSEWLPGGYLNSAKNCLNVNSNKKLNDTMIVWRDEGNDDLPL
NKLTLDQLRKRVWLVGYALEEMGLEKGCAIAIDMPMHVDAVVIYLAIVLAGYVVVSIADSFSAPEISTRL
RLSKAKAIFTQDHIIRGKKRIPLYSRVVEAKSPMAIVIPCSGSNIGAELRDGDISWDYFLERAKEFKNCE
FTAREQPVDAYTNILFSSGTTGEPKAIPWTQATPLKAAADGWSHLDIRKGDVIVWPTNLGWMMGPW
LVYASLLNGASIALYNGSPLVSGFAKFVQDAKVTMLGVVPSIVRSWKSTNCVSGYDWSTIRCFSSSG
EASNVDEYLWLMGRANYKPVIEMCGGTEIGGAFSAGSFLQAQSLSSFSSQCMGCTLYILDKNGYPM
PKNKPGIGELALGPVMFGASKTLLNGNHHDVYFKGMPTLNGEVLRRHGDIFELTSNGYYHAHGRAD
DTMNIGGIKISSIEIERVCNEVDDRVFETTAIGVPPLGGGPEQLVIFFVLKDSNDTTIDLNQLRLSFNLGL
QKKLNPLFKVTRVVPLSSLPRTATNKIMRRVLRQQFSHFE
SEQ ID NO: 26-TKS candidate isolated from Dendrobium catenatum
Amino acid sequence
MPSLESIRKAPRANGFASILAIGRANPENFIEQSTYPDFFFRITNSEHLVDLKKKFQRICDKTAIRKRHF
VWNEEFITTNPCLHTFMDKSLDVRQEVAIREIPKLGAKAAAKAIQEWGQPKSRITHLIFCTTSGMDLPG
ADYQLTQILGLNPNVERVMLYQQGCFAGGTTLRLAKCLAESRKGARVLVVCAETTTVLFRGPSEEHQ
EDLVTQALFADGASALIVGADPDEAAHERASFVIVSTSQVLLPDSAGAIGGHVSEGGLLATLHRDVPKI
VSKNVEKCLEEAFTPFGITDWNSIFWVPHPGGRAILDLVEERVGLKPEKLLVSRHVLAEYGNMSSVCV
HFALDEMRKRSAIEGKATTGEGLEWGVVFGFGPGLTVETVVLRSVPL
SEQ ID NO: 27-TKS candidate isolated from Dictyostelium
Amino acid sequence
MNNSNVKSSPSIVKEEIVTLDKDQQPLLLKEHQHIIISPDIRINKPKRESLIRTPILNKFNQITESIITPSTPS
LSQSDVLKTPPIKSLNNTKNSSLINTPPIQSVQQHQKQQQKVQVIQQQQQPLSRLSYKSNNNSFVLGI
GISVPGEPISQQSLKDSISNDFSDKAETNEKVKRIFEQSQIKTRHLVRDYTKPENSIKFRHLETITDVNN
QFKKVVPDLAQQACLRALKDWGGDKGDITHIVSVTSTGIIIPDVNFKLIDLLGLNKDVERVSLNLMGCL
AGLSSLRTAASLAKASPRNRILVVCTEVCSLHFSNTDGGDQMVASSIFADGSAAYIIGCNPRIEETPLY
EVMCSINRSFPNTENAMVWDLEKEGWNLGLDASIPIVIGSGIEAFVDTLLDKAKLQTSTAISAKDCEFLI
HTGGKSILMNIENSLGIDPKQTKNTWDVYHAYGNMSSASVIFVMDHARKSKSLPTYSISLAFGPGLAF
EGCFLKNVV
SEQ ID NO: 28-TKS candidate isolated from Arachis hypogaea
Amino acid sequence
MNNSNVKSSPSIVKEEIVTLDKDQQPLLLKEHQHIIISPDIRINKPKRESLIRTPILNKFNQITESIITPSTPS
LSQSDVLKTPPIKSLNNTKNSSLINTPPIQSVQQHQKQQQKVQVIQQQQQPLSRLSYKSNNNSFVLGI
GISVPGEPISQQSLKDSISNDFSDKAETNEKVKRIFEQSQIKTRHLVRDYTKPENSIKFRHLETITDVNN
QFKKVVPDLAQQACLRALKDWGGDKGDITHIVSVTSTGIIIPDVNFKLIDLLGLNKDVERVSLNLMGCL
AGLSSLRTAASLAKASPRNRILVVCTEVCSLHFSNTDGGDQMVASSIFADGSAAYIIGQNPRIEETPLY
EVMCSINRSFPNTENAMVWDLEKEGWNLGLDASIPIVIGSGIEAFVDTLLDKAKLQTSTAISAKDCEFLI
HTGGKSILMNIENSLGIDPKQTKNTWDVYHAYGNMSSASVIFVMDHARKSKSLPTYSISLAFGPGLAF
EGCFLKNVV
SEQ ID NO: 29-TKS candidate isolated from Spinacia oleracea
Amino acid sequence
MASVDISEIHNVERAKGQANVLAIGTANPPNVMYQADYPDFYFRLTNSEHMTDLKAKFKRICEKTTIKK
RYMHISEDILKEKPDLCDYNASSLDIRQVILAKEVPKVGKDAAMKAIEEWGQAMSKITHLIFCTTSGVDI
PGADYQLTMLLGLNPSVKRYMLCQQGCHAGGTVLRLAKDLAENNYGSRVLVVCSENTTVCFRGPTE
THPDSMVAQALFADGAGAVIVGAYPDESLNERPIFQIVSTAQTILPNSQGAIEGHLRQIGLAIQLLPNVP
DLISNNIDKCLVEAFNPIGINDWNSIFWIAHPGGPAILGQVESKLGLQESKLTTTWHVLREFGNMSSAC
VFFIMDETRKRSLKEGKTTTGDGFDWGVLFGFGPGLTVETVVLRSFPLNQ
SEQ ID NO: 30-TKS candidate isolated from Elaeis guineensis
Amino acid sequence
MSGLSRDMNPSLERSVGRAAVLGIGTANPPHVVEQSTFPDYYFKITNSEHMAHLKEKFTRICEKSKIA
KRYTVLTDEFLVANPTLTSFNAPSLDTRQQLLDVEVPRLGAEAATRAIKDWGRPMSDLTHLIFCNSYG
ASIPGADYELVKLLGLPLSTRRVMLYQQCCYAGGTVIRLAKDLAENNRDARVLVVCCELNTVGIRGPC
QSHLEDLVSQALFGDGAGALIIGADPRAGVERSIFEIVRTSQNIIAGSEGALVAKLREVGLVGRLKPEIP
MHLSCSIEKLASEALNPVGIADWNEAFWVMHPGGRAILDELEKKLGLGEEKLAATREVLRDYGNMSS
TSVLFVMEVMRRRSEERGLATAGEGLEWGVLLGFGPGLTMETVVLRCP
SEQ ID NO: 31-TKS candidate isolated from Vitis pseudoreticulata
Amino acid sequence
MALVEEIRNAQRAKGPATVLAIGTATPDNCLYQSDFADYYFRVTKSEHMTELKKKFNRICDKSMIKKR
YIHLTEEMLEEHPNIGAYMAPSLNIRQEIITAEVPKLGKEAALKALKEWGQPKSKITHLVFCTTSGVEMP
GADYKLANLLGLEPSVRRVMLYHQGCYAGGTVLRTAKDLAENNAGARVLVVCSEITVVTFRGPSENA
LDSLVGQALFGDGSAAVIVGSDPDISIERPLFQLVSAAQTFIPNSAGAIAGNLREVGLTFQLWPNVPTLI
SENIEKCLTKAFDPIGISDWNSLFWIAHPGGPAILDAVEAKLNLDKQKLKATRHILSEYGNMSSACVLFI
LDEMRKKSLKEGKTTTGEGLDWGVLFGFGPGLTIETVVLHSVQMDSN
SEQ ID NO: 32-TKS candidate isolated from Cannabis sativa
Amino acid sequence
MASISVDQIRKAQRANGPATVLAIGTANPPTSFYQADYPDFYFRVTKNQHMTELKDKFKRICEKTTIKK
RHLYLTEDRLNQHPNLLEYMAPSLNTRQDMLVVEIPKLGKEAAMKAIKEWGQPKSRITHLIFCSTNGV
DMPGADYECAKLLGLSSSVKRVMLYQQGCHAGGSVLRIAKDLAENNKGARILTINSEITIGIFHSPDET
YFDGMVGQALFGDGASATIVGADPDKEIGERPVFEMVSAAQEFIPNSDGAVDGHLTEAGLVYHIHKD
VPGLISKNIEKSLVEALNPIGISDWNSLFWIVHPGGPAILNAVEAKLHLKKEKMADTRHVLSEYGNMSS
VSIFFIMDKLRKRSLEEGKSTTGDGFEWGVLFGFGPGLTVETIVLHSLAN
SEQ ID NO: 33-TKS candidate isolated from Chenopodium quinoa
Amino acid sequence
MASVQEIRNAQRADGPATILAIGTANPPNEMYQAEYPDFYFRVTESEHMTDLKKKFKRMCERSMIKK
RYMHVTEELLKENPHMCDYNASSLNTRQDILATEVPKLGKEAAIKAIKEWGQPRSKITHVIFCTTSGVD
MPGADYQLTKLLGLRPSVKRFMLYQQGCYAGGTVLRLAKDIAENNRGARVLVVCAEITVICFRGPTET
HLDSMIGQALFGDGAGAVIVGADVDESIERPIFQLVWAAQTILPDSEGAIDGHLREVGLAFHLLKDVPG
LISKNIEKALVEAFKPIGIDDWNSIFWVAHPGGPAILDQVESKLELKQDKLRDTRHVLSEFGNMSSACV
LFILDEMRNRSLKEGKTTTGEGLDWGVLFGFGPGLTVETVMLHSVPITN
SEQ ID NO: 34-TKS candidate isolated from Ziziphus jujuba
Amino acid sequence
MVTVDEIREAQRAKGPATIMAIGTATPPNAIDQSTFTDYYFRITNSDHKTDLKKKFKTICDKSMIKKRYL
YLTEEHLKQNPNMSEYMAPSLDVRQEIVIAEVPKLGKEAANKAIKEWGQPKSKITHLVFSTISGVDAPG
ADYQLTKLLGLNPSVKRIMVYQQGCFAGGTSLRLAKDLAENNKGARVLVVCTEISAINFRGPSETYFD
SNVGQILFGDGASAVVVGSDPLVGVEKPLFELVSASQTIIPDSEGNIEGHICEVGLTIRLSKKVPSLISN
NIEKSLVEAFNPLGISDWNSIFWIAHPGGPAILDQIELKLGLKPEKLRASRHVLSEYGNMSSATVLFILD
EMRKKSIEDGLKTPGEGLEWGVLFGFGPGLTVETVVLHSVTA
SEQ ID NO: 35-TKS candidate isolated from Marchantia polymorpha
Amino acid sequence
MSRSRLIAQAVGPATVLAMGKAVPANVFEQATYPDFFFNITNSNDKPALKAKFQRICDKSGIKKRHFY
LDQKILESNPAMCTYMETSLNCRQEIAVAQVPKLAKEASMNAIKEWGRPKSEITHIVMATTSGVNMPG
AELATAKLLGLRPNVRRVMMYQQGCFAGATVLRVAKDLAENNAGARVLAICSEVTAVTFRAPSETHI
DGLVGSALFGDGAAAVIVGSDPRPGIERPIYEMHWAGEMVLPESDGAIDGHLTEAGLVFHLLKDVPG
LITKNIGGFLKDTKNLVGASSWNELFWAVHPGGPAILDQVEAKLELEKG
SEQ ID NO: 36-TKS candidate isolated from Caragana korshinskii
Amino acid sequence
MAYLEEIREVQRARGPATILAIGTANPSNCIYQADFTDYYFRVTNSDHMTKLKAKLKRICENSMIKKRH
VHLTEEILKENPNICTYKESSLDARQDMLIVEVPKLGEKAASKAIEEWGRPKSEITHLIFCSTSGVDMP
GADYQLINLLGLKPSTKRFMLYHQGCFAGGTVLRLAKDLAENNAGARVLVVCSEITVVTFRGPSETHL
DCLVGQALFGDGASSVIVGSDPDTSIERPLFHLVSASETILPNSEGAIEGHLREAGLMFQLKENVPQLI
GENIEKSLEEMFHPLGISDWNSLFWISHPGGPAILKRIEETAGLNPEKLKATKHVLSEYGNMSSACVLF
ILDEMRKRSMEEGKSTTGEGLNWGVLFGFGPGLTMETIALHSANIDTGY
SEQ ID NO: 37-TKS candidate isolated from Glycine max
Amino acid sequence
MVSVAEIRQAQRAEGPATILAIGTANPPNCVAQSTYPDYYFRITNSEHMTELKEKFQRMCDKSMIKRR
YMYLNEEILKENPNMCAYMAPSLDARQDMVVVEVPKLGKEAAVKAIKEWGQPKSKITHLIFCTTSGVD
MPGADYQLTKQLGLRPYVKRYMMYQQGCFAGGTVLRLAKDLAENNKGARVLVVCSEITAVTFRGPS
DTHLDSLVGQALFGDGAAAVIVGSDPIPQVEKPLYELVWTAQTIAPDSEGAIDGHLREVGLTFHLLKDV
PGIVSKNIDKALFEAFNPLNISDYNSIFWIAHPGGPAILDQVEQKLGLKPEKMKATRDVLSEYGNMSSA
CVLFILDEMRRKSAENGLKTTGEGLEWGVLFGFGPGLTIETVVLRSVAI
SEQ ID NO: 38-TKS candidate isolated from Humulus lupulus
Amino acid sequence
MASVTVEQIRKAQRAEGPATILAIGTAVPANCFNQADFPDYYFRVTKSEHMTDLKKKFQRMCEKSTIK
KRYLHLTEEHLKQNPHLCEYNAPSLNTRQDMLVVEVPKLGKEAAINAIKEWGQPKSKITHLIFCTGSSI
DMPGADYQCAKLLGLRPSVKRVMLYQLGCYAGGKVLRIAKDIAENNKGARVLIVCSEITACIFRGPSE
KHLDCLVGQSLFGDGASSVIVGADPDESVGERPIFELVSAAQTILPNSDGAIAGHVTEAGLTFHLLRDV
PGLISQNIEKSLIEAFTPIGINDWNNIFWIAHPGGPAILDEIEAKLELKKEKMKASREMLSEYGNMSCAS
VFFIVDEMRKQSSKEGKSTTGDGLEWGALFGFGPGLTVETLVLHSVPTNV
SEQ ID NO: 39-TKS candidate isolated from Humulus lupulus
Amino acid sequence
MVTVEEVRKAQRAEGPATILAIGTATPANCILQSEYPDYYFRITNSEHKTELKEKFKRMCDKSMIRKRY
MHLTEEILKENPNLCAYEAPSLDARQDMVVVEVPKLGKEAATKAIKEWGQPKSKITHVVFCTTSGVD
MPGADYQLTKLLGLRPSVKRLMMYQQGCFAGGTVLRVAKDLAENNKGARVLVVCSEITAVTFRGPN
DTHLDSLVGQALFGDGSAALIIGADPTPEIEKPIFELVSAAQTILPDSDGAIDGHLREVGLTFHLLKDVP
GLISKNIEKSLVEAFKPLGISDWNSLFWIAHPGGPAILDQVESKLALKPEKLRATRHVLGEYGNMSSAC
VLFILDEMRRKCAEDGLKTTGEGLEWGVLFGFGPGLTVETVVLHSVGI
SEQ ID NO: 40-TKS candidate isolated from Trema orientale
Amino acid sequence
MASVTVDEIRKAQRAEGPATVLAIGTATPHNCVSQADYPDYYFRITNSEHMTELKEKFKRMCEKSMIK
KRYMHLTEEILKENPKMCEYMAPSLDARQDMVVVEVPKLGKEAATKAIKEWGLPKSKITHLVFCTTSG
VDMPGADYQLTKLLGLRPSVKRLMMYQQGCFAGGTVLRLAKDLAENNRGARVLVVCSEITAVTFRG
PSDTHLDSMVGQALFGDGAAAVIVGADPDPSAGERPLFEMVSAAQTILPDSEGAIDGHLREAGLTFH
LLKDVPGLISKNIEKSLTEAFSPLGISDWNSLFWIAHPGGPAILDQVEAKLKLKEEKLRATRHVLSEYGN
MSSACVLFILDEMRKKSAEDGKPTTGEGLDWGVLFGFGPGLTVETVVLHSVAATATN
SEQ ID NO: 41-TKS candidate isolated from Plumbago indica
Amino acid sequence
MAPAVQSQSHGGAYRSNGERSKGPATVLAIATAVPPNVYYQDEYADFFFRVTNSEHKTAIKEKFNRV
CGTSMIKKRHMYFTEKMLNQNKNMCTWDDKSLNARQDMVIPAVPELGKEAALKAIEEWGKPLSNITH
LIFCTTAGNDAPGADFRLTQLLGLNPSVNRYMIYQQGCFAGATALRIAKDLAENNKGARVLIVCCEIFA
FAFRGPHEDHMDSLICQLLFGDGAAAVIVGGDPDETENALFELEWANSTIIPQSEEAITLRMREEGLMI
GLSKEIPRLLGEQIEDILVEAFTPLGITDWSSLFWIAHPGGKAILEALEKKIGVEGKLWASWHVLKEYGN
LTSACVLFAMDEMRKRSIKEGKATTGDGHEYGVLFGVGPGLTVETVVLKSVPLN
SEQ ID NO: 42-TKS candidate isolated from Artemisia annua
Amino acid sequence
MASLTDIAAIREAQRAQGPATILAIGTANPANCVYQADYPDYYFRITKSEHMVDIKEKFKRMCDKSMIR
KRYMHLTEEYLKENPSLCEYMAPSLDARQDVVVVEVPKLGKEAATKAIKEWGQPKSKITHLIFCTTSG
VDMPGADYQLTKLLGLRPSVKRFMMYQQGCFAGGTVLRLAKDLAENNKDARVLVVCSEITAVTFRG
PNDTHLDSLVGQALFGDGAAAVIVGSDPDLTKERPLFEMISAAQTILPDSEGAIDGHLREVGLTFHLLK
DVPGLISKNIEKALTQAFSPLGISDWNSIFWIAHPGGPAILDQVELKLGLKEEKMRATRHVLSEYGNMS
SACVLFIIDEMRKKSAEEGAATTGEGLDWGVLFGFGPGLTVETVVLHSLPTTISVVN
SEQ ID NO: 43-TKS candidate isolated from Actinidia chinensis var. chinensis
Amino acid sequence
MAPSLEEILRAQRSQGPAEILGIGTATPPNCYDQADFPDFYFRVTNSEHMTHLKDKFKQICEKSTVKK
RYMYLTEEILKDNPSLCSYMGRSLDVRQNMVMTEVPKLGKEAAAKAIKEWGQPKSKITHLVFCTTSG
VDMPGADYHLTKLLGLQPSVKRIMMYQSSCYGGGTGLRLAKDLAENNAGARVLLVCSEISAINFRGP
PDTPARLDKLVAQALFGDGAAAVIVGADPDTSIERSLFQLISASQTIVPGSNGGIMGTFGEAGLMCHLI
KDVPRLISSNIEKCLMDAFTPIGINDWNSIFWIAHPGGPAILDMVEEKIGLEEEKLRATRHILSEYGNMS
SVCVLFILDEMRKKSAEEGKLTTGEGLEWGVLFGFGAGITVETVVLRSMSISNTTH
SEQ ID NO: 44-TKS candidate isolated from Rhododendron dauricum
Amino acid sequence
MVTVEDVRKAQRAEGPATVMAIGTATPPNCVDQSTYPDFYFRITNSEHKAELKEKFQRMCDKSMIKK
RYMYLTEEILKENPSVCEYMAPSLDARQDMVVVEVPKLGKEAATKAIKEWGQPKSKITHLVFCTTSGV
DMPGADYQLTKLLGLRPSVKRLMMYQQGCFAGGTVLRLAKDLAENNKGARVLVVCSEITAVTFRGP
SDTHLDSLVGQALFGDGAAAIIVGADPVPEVEKPLFELVSAAQTILPDSDGAIDGHLREVGLTFHLLKD
VPGLISKNIEKALTEAFQPLGISDWNSIFWIAHPGGPAILDQVELKLSLKPEKLRATRHVLSEYGNMSSA
CVLFILDEMRRKSAEEGLKTTGEGLEWGVLFGFGPGLTVETVVLHSLCT
SEQ ID NO: 45-TKS candidate isolated from Chenopodium quinoa
Amino acid sequence
MASASMNPATILAIGTANPPNVMCQSDYPDYHFRTTNSDHLTDLKAKFKRICDKSMIRKRHFYMNEEI
LKENPHLGDNNASSIGTRQALCANEIPKLGKEAAEKAIKEWGKPKSMITHLIFGTNSDFDLPGADFRLA
KLLGLQPTVKRFILPLGACHAGGTALRIAKDIAENNRGARVLVICSESTAISFHAPSETHLVSLAIFGDG
AGAMIVGTDPDEPSERPLFQLVSAGQITLPDSEDGIQARLSEIGMTIHLSPDVPKIIAKNIQTLLSESFDH
IGISNWNSIFWVAHPGGPAILDKVEAKLELETSKLSTSRHILSEYGNMWGASVIFVMDEMSKRSLKEG
KSTTGEGCEWGVLVAFGPGITVETIVLRSMPINY
SEQ ID NO: 46-TKS candidate isolated from Cajanus cajan
Amino acid sequence
MVSVEDIRKAQRAEGPATVMAIGTATPPNCVDQSTYPDYYFRITNSEHKTELKEKFKRMCDKSMIKKR
YMYLNEEILKENPSVCEYMAPSLDARQDMVVVEVPKLGKEAATKAIKEWGQPKSKITHLIFCTTSGVD
MPGADYQLTKLLGLRPSVKRYMMYQQGCFAGGTVLRLAKDLAENNKGARVLVVCSEITAVTFRGPS
DTHLDSLVGQALFGDGAAAVIVGSDPLPVEKPFFELVWTAQTILPDSEGAIDGHLREVGLTFHLLKDV
PGLISKNIEKALVEAFQPLGISDYNSIFWIAHPGGPAILDQVEAKLGLKPEKMEATRHVLSEYGNMSSA
CVLFILDQMRKKSIENGLGTTGEGLEWGVLFGFGPGLTVETVVLRSVTV
SEQ ID NO: 47-TKS candidate isolated from Lonicera japonica
Amino acid sequence
MGSVTVEEIRKAQRAQGPATVLAIGTATPANCVYQADYPDFYFRITKSEHKAELKEKFKRMCEKSMIR
KRYMHLNEEILKENPGICEYMAPSLDARQDMVVVEVPKLGKEAATKAIKEWGQPKSKITHLVFCTTSG
VDMPGADYQLTKLLGLRPSVKRLMMYQQGCFAGGTVLRLAKDLAENNAGARVLVVCSEITAVTFRG
PSDTHLDSLVGQALFGDGAAAVIIGADPDKSVERPLFELVSAAQTILPDSDGAIDGHLREVGLTFHLLK
DVPGLISKNIEKSLKEAFAPIGITDWNSLFWIAHPGGPAILDQVEIKLGLKEEKLRPTRHVLSEYGNMSS
ACVLFILDELRKKSIEEGKATTGDGLEWGVLFGFGPGLTVETVVLHSVPASI
SEQ ID NO: 48-TKS candidate isolated from Ruta graveolens
Amino acid sequence
MESLKEMRKAQMSEGPAAILAIGTATPNNVYMQADYPDYYFRMTKSEHMTELKDKFRTLCEKSMIRK
RHMCFSEEFLKANPEVSKHMGKSLNARQDIAVVETPRLGNEAAVKAIKEWGQPKSSITHLIFCSSAGV
DMPGADYQLTRILGLNPSVKRMMVYQQGCYAGGTVLRLAKDLAENNKGSRVLVVCSELTAPTFRGP
SPDAVDSLVGQALFADGAAALVVGADPDSSIERALYYLVSASQMLLPDSDGAIEGHIREEGLTVHLKK
DVPALFSANIDTPLVEAFKPLGISDWNSIFWIAHPGGPAILDQIEEKLGLKEDKLRASKHVMSEYGNMS
SSCVLFVLDEMRSRSLQDGKSTTGEGLDWGVLFGFGPGLTVETVVLRSVPIEA
SEQ ID NO: 49-TKS candidate isolated from Physcomitrella patens subsp. patens
Amino acid sequence
MASAGDVTRAALPRAQPRAEGPACVLGIGTAVPPAEFLQSEYPDFFFNITNCGEKEALKAKFKRICDK
SGIRKRHMFLTEEVLKANPGICTYMEPSLNVRHDIVVVQVPKLAAEAAQKAIKEWGGRKSDITHIVFAT
TSGVNMPGADHALAKLLGLKPTVKRVMMYQTGCFGGASVLRVAKDLAENNKGARVLAVASEVTAVT
YRAPSENHLDGLVGSALFGDGAGVYVVGSDPKPEVEKPLFEVHWAGETILPESDGAIDGHLTEAGLIF
HLMKDVPGLISKNIEKFLNEARKPVGSPAWNEMFWAVHPGGPAILDQVEAKLKLTKDKMQGSRDILS
EFGNMSSASVLFVLDQIRHRSVKMGASTLGEGSEFGFFIGFGPGLTLEVLVLRAAPNSA
SEQ ID NO: 50-TKS candidate isolated from Rubus idaeus
Amino acid sequence
MGSVAKEAKYPATILAIATANPANCYHQKDYPDFLFRVTKSEDKTELKDKFKRICEKSMVKKRYLGITE
ESLNANPNICTYKAPSLDSRQDLLVHEVPKLGKEAALKAIEEWGQPISSITHLIFCTASCVDMPGADFQ
LVKLLGLDPTIKRFMIYQQGCFAGGTVLRIAKDVAENNAGARLLIVCCEITTMFFQQPSENHLDVLVGQ
ALFSDGAAALIVGTNPDPKSERQLFDIMSVRETIIPNSEHGVVAHLREMGFEYYLSSEVPKLVGGKIEE
YLNKGFEGIGVDGDWNSLFYSIHPGGPAILNKVEEELGLKEGKLRATRHVLSEFGNMGAPSVLFILDEI
RKRSMEEGKATTGEGFEWGVLIGIGPGLTVETVVLRSVSTAN
SEQ ID NO: 51-TKS candidate isolated from Marchantia polymorpha subsp. ruderalis
Amino acid sequence
MATRVLSSQENFEKLMADLARPNGHVYSQSQSQSGSGQNGAGTSIVAKNTASILAIGKALPPNRICQ
STYTDFYFRVTHCSHKTELKNRMQRICDKSGINTRYLLLDEEALKEHSEFYTPGQASIEQRHDLLEEA
VPKLAAQAAASALEEWGRPACDVTHLIVVTLSGVAIPGADVRLVKLLGLREDVSRVMLYMLGCYAGV
TALRLAKDLAENNPGSRVLIACSEMTATTFRAPSEKSMYDIVGASLFGDGAVGVIVGAKPRPGIERSIF
EIHWAGVSLAPDTEHVVQGKLKPDGLYFFLDKSLPGLVGKHIAPFCRSLLDHAPENLNLGFNEVFWA
VHPGGPAILNTVEEQLLLNSEKLRASRDVLANYGNVSASSVLYVLDELRHRPGQEEWGAALAFGPGI
TFEGVLLRRNVNHR
SEQ ID NO: 52-TKS candidate isolated from Oryza sativa
Amino acid sequence
MGKQGGQQLVAAILGIGTAVPPYVLPQSSFPDYYFDISNSNHLLDLKAKFADICEKTMIDKRHVHMSD
EFLRSNPSVAAYNSPSINVRQNLTDVTVPQLGAAAARLAIADWGRPACEITHLVMCTTVSGCMPGAD
FEVVKLLGLPLTTKRCMMYHIGCHGGGTALRLAKDLAENNPGGRVLVVCSEVVSMVFRGPCESHMG
NLVGQALFGDAAGAVVVGADPVEANGERTLFEMVSAWQDIIPETEEMVVAKLREEGLVYNLHRDVAA
RVAASMESLVKKAMVEKDWNEEVFWLVHPGGRDILDRVVLTLGLRDDKVAVCREVMRQHGNTLSS
CVIVAMEEMRRRSADRGLSTAGEGLEWGLLFGFGPGLTVETILLRAPPCNQAQAV
SEQ ID NO: 53-TKS candidate isolated from Punica granatum
Amino acid sequence
MGYSQQAKGPATIMAIGTAIPSYVVYQADFPDYYFRLSGCDHMTELKEKFIRICEKSTIRKRHMHLTEE
ILKQNPAILTYDGPSLNVRQQLVASEVPKLAMEAASKAIEEWGQPVWKITHLVFSSVVGAATPGADYK
LIKLLGLEPSVKRVPLYQQGCYVGGTALRIAKDLAENNASARVLVVCVDNTISSFRGPSKHITNLVGQA
LFSDGASAAIVGADPIPSVERPIFQIAHTSMHLVPDSDSEVTLDFLDAGLIVHVSEKVPSLIADNLEKSLV
EALGPTGINDWNSLFWAAHPGGPKILDMIEAKLGLRKEKLRATRTVLREYGNMIGACLLFILDEIRQNS
LEAGMATTGEGFDWGILLGFGPGLTVEAVVLRSFPIAK
SEQ ID NO: 54-TKS candidate isolated from Citrus x microcarpa
Amino acid sequence
MAKVKNFLNAKRSKGPASILAIGTANPPTCFNQSDYPDFYFRVTDCEHKTELKDKFKRICDRSAVKKR
YLHVTEEVLKENPSMRSYNAPSLDARQALLIEQVPKLGKEAAAKAIKEWGQPLSKITHLVFSAMAGVDI
PGADLRLMNLLGLEPSVKRLMIYSQGCFIGGAAIRCAKDFAENNAGARVLVVFSDIMNMYFHEPQEA
HLDILVGQAVFGDGAAAVIVGADPEVSIERPLFHVVSSTQMSVPDTNKFIRAHVKEMGMELYLSKDVP
ATVGKNIEKLLVDAVSPFGISDWNSLFYSVHPGGRAILDQVELNLGLGKEKLRASRHVLSEYGNMGG
SSVYFILDEIRKKSMQEAKPTTGDGLEWGVLFAIGPGLTVETVILLSVPIDSAC
SEQ ID NO: 55-TKS candidate isolated from Rhododendron dauricum
Amino acid sequence
MALVNHRENVKGRAQILAIGTANPKNCFRQVDYPDYYFRVTKSDHLIDLKAKFKRMCEKSMIEKRYM
HVNEEILEQNPSMNHGGEKMVSSLDVRLDMEIMEIPKLAAEAATKAMDEWGQPKSRITHLVFHSTLG
TVMPGVDYELIKLLGLNPSVKRFMLYHLGCYGGGTVLRLAKDLAENNPGSRVLVLCCEMMPSGFHG
PPSLQHAHLDILTGHAIFGDGAGAVIVGCVDPSGGTNGVVERGVRRYEQPLFEIHSAYQTVLPDSKDA
VGGRLREAGLIYYLSKRLSNDVSGKIDECCLAEAFSAAIKDNFEDWNSLFWIVHPAGRPILDKLDAKLG
LNKEKLRASRNVLRDYGNMWSSSVLFVLDEMRKGSIAQRKTTTGEGFEWGVLLGFGPGVTVETVVL
RSVPTAKLK
SEQ ID NO: 56-TKS candidate isolated from Curcuma zedoaria
Amino acid sequence
MEANGYRITHSADGPATILAIGTANPTNVVDQNAYPDFYFRVTNSEHLQELKAKFRRICEKAAIRKRHL
YLTEEILRENPSLLAPMAPSFDARQAIVVEAVPKLAKEAAEKAIKEWGRPKSDITHLVFCSASGIDMPG
SDLQLLKLLGLPPSVNRVMLYNVGCHAGGTALRVAKDLAENNRGARVLAVCSEVTVLSYRGPHPAHI
ESLFVQALFGDGAAALVVGSDPVDGVERPIFEIASASQVMLPESEEAVGGHLREIGLTFHLKSQLPSII
ASNIEQSLTTACSPLGLSDWNQLFWAVHPGGRAILDQVEARLGLEKDRLAATRHVLSEYGNMQSATV
LFILDEMRNRSAAEGHATTGEGLDWGVLLGFGPGLSIETVVLHSCRLN
SEQ ID NO: 57-TKS candidate isolated from Garcinia mangostana
Amino acid sequence
MAPAMDSAQNGHQSRGSANVLAIGTANPPNVILQEDYPDFYFKVTNSEHLTDLKEKFKRICVKSKTR
KRHFYLTEQILKENPGIATYGAGSLDSRQKILETEIPKLGKEAAMVAIQEWGQPVSKITHVVFATTSGF
MMPGADYSITRLLGLNPNVRRVMIYNQGCFAGGTALRVAKDLAENNKGARVLVVCAENTAMTFHGP
NENHLDVLVGQAMFSDGAAALIIGANPNLPEERPVYEMVAAHQTIVPESDGAIVAHFYEMGMSYFLKE
NVIPLFGNNIEACMEAAFKEYGISDWNSLFYSVHPGGRAIVDGIAEKLGLDEENLKATRHVLSEYGNM
GSACVIFILDELRKKSKEEKKLTTGDGKEWGCLIGLGPGLTVETVVLRSVPIA
SEQ ID NO: 58-TKS candidate isolated from Arachis hypogaea
Amino acid sequence
MGSLGATQEGNGAKGVATILAIGTANPPNIIRQDDYPDFYFRATKSNHMLHLKEKFQRLCKNSMIEKR
HFLYNEDLLMENPNIVTYGASSLNTRQNILIKEVPKLGKEAALKAINEWGQPLSEITHLIFYTTSCFGNM
PGPDYHLAKLLGLKPTVNRHMIFNNGCHGGGAVLRVAKDIVENNAGSRVLVVWVETMVASFHGPNP
NHMDVLVGQALFGDGAGALIIGTNPKPCIECPLFELVLASQTTIPNTESSINGNIQEMGLVYYLGKEIPIA
ISENIDKCLINAFRESSVDWNSLFYAIHPGGPSILNRIEEKLGLKKEKLRASRKVLSQYGNMWSPGVIFV
LDELRNWSKIEGKSTCGEGKEWGVLVGFGPGLSLELLVLRSFCFDG
SEQ ID NO: 59-TKS candidate isolated from Aquilaria sinensis
Amino acid sequence
MAAQPVEWVRKADRAAGPAAVLAMATANPSNFYLQSDFPDFYFRVTRSDHMSDLKEKFKRICKKTT
VRKRHMILTEEILNKNPAIADYWSPSLAARHDLALANIPQLGKEAADKAIKEWGQPKSKITHLVFCTSA
GVLMPGADYQLTMLLGLNPSISRLMLHNLGCYAGGTALRVAKDLAENNGGARVLVVCSEANLLNFRG
PSETHIDALITQSLFADGAAALIVGSDPDLQTESPLYELISASQRILPESEDAIVGRLTEAGLVPYLPKDI
PKLVSTNIRSILEDALAPTGVQDWNSIFWIIHPGMPAILDQTEKLLQLDKEKLKATRHVLSEFGNMFSAT
VLFILDQLRKGAVAEGKSTTGEGCEWGVLFSFGPGFTVETVLLRSVATATLTDA
SEQ ID NO: 60-TKS candidate isolated from Cs.
Amino acid sequence
MNHLRAEGPASVLAIGTANPENILLQDEFPDYYFRVTKSEHMTQLKEKFRKICDKSMIRKRNCFLNEE
HLKQNPRLVEHEMQTLDARQDMLVVEVPKLGKDACAKAIKEWGQPKSKITHLIFTSASTTDMPGADY
HCAKLLGLSPSVKRVMMYQLGCYGGGTVLRIAKDIAENNKGARVLAVCCDIMACLFRGPSESDLELL
VGQAIFGDGAAAVIVGAEPDESVGERPIFELVSTGQTILPNSEGTIGGHIREAGLIFDLHKDVPMLISNNI
EKCLIEAFTPIGISDWNSIFWITHPGGKAILDKVEEKLHLKSDKFVDSRHVLSEHGNMSSSTVLFVMDE
LRKRSLEEGKSTTGDGFEWGVLFGFGPGLTVERVVVRSVPIKY
SEQ ID NO: 61-CBGaS candidate isolated from Sb.PT (A0A193PS58)
Amino acid sequence
MPATRTPIHPEAAAYKNPRYQSGPLSVIPKSFVPYCELMRLELPHGNFLGYFPHLVGLLYGSSASPAR
LPANEVAFQAVLYIGWTFFMRGAGCAWNDVVDQDFDRKTTRCRVRPVARGAVSTTSANIFGFAMVA
LAFACISPLPAECQRLGLMTTVLSIIYPFCKRVTNFAQVILGMTLAINFILAAYGAGLPAIEAPYTVPTICV
TTAITLLVVFYDVVYARQDTADDLKSGVKGMAVLFRNYVEILLTSITLVIAGLIATTGVLVDNGPYFFVFS
VAGLLAALLAMIGGIRYRIFHTWNSYSGWFYALAIFNLLGGYLIEYLDQVPMLNKA
SEQ ID NO: 62-CBGaS candidate isolated from Sc.PT (A0A084RYZ7)
Amino acid sequence
MSAKVSPMAYTNPRYETGPLSLIPKPIVPYFELMRFELPHGYYLGYFPHLVGIMYGASAGPERLPARD
LVFQALLYVGWTFAMRGAGCAWNDNIDQDFDRKTERCRTRPIARGAVSTTAGHVFAVAGVALAFLC
LSPLPTECHQLGVLVTVLSVIYPFCKRFTNFAQVILGMTLAANFILAAYGAGLPALEQPYTRPTMSATL
AITLLVVFYDVVYARQDTADDLKSGVKGMAVLFRNHIEVLLAVLTCTIGGLLAATGVSVGNGPYYFLFS
VAGLTVALLAMIGGIRYRIFHTWNGYSGWFYVLAIINLMSGYFIEYLDNAPILARGS
SEQ ID NO: 63-CBGaS candidate A0A084B1B1
Amino acid sequence
MSAKVSPMAYTNPRYERGPLSLIPKPIVPYFELMRFELPHGYYLGYFPHLVGIMYGASAGPERLPARD
LVFQALLYVGWTFAMRGAGCAWNDNIDQDFDRKTERCRTRPIARGAVSTTAGHVFAVAGVALAFLC
LSPLPTECHQLGVLVTVLSVIYPFCKRFTNFAQVILGMTLAANFILAAYGAGLPALEQPYTRPTMSATL
AITLLVVFYDVVYARQDTADDLKSGVKGMAVLFRNHIEVLLAVLTCTIGGLLAATGVSVGNGPYYFLFS
VAGLTVALLAMIGGIRYRIFHTWNGYSGWFYVLAIINLMSGYFIEYLDNAPILARGS
SEQ ID NO: 64-CBGaS candidate A0A084QZF6
Amino acid sequence
MSPKVSSMPYTNPRYESGPLSLIPKSIVPYFELMRFELPHGYYLGYFPHLVGIMYGASAGPERLPARD
LVFQALLYVGWTFAMRGAGCAWNDNIDQDFDRKTERCRTRPIARGAVSTTAGHIFAVAGVALAFLCL
SPLPTECHQLGVLVTVLSVIYPFCKRFTNFAQVILGMTLAANFILAAYGAGLPALEQPYTRPTMFATLAI
TLLVVFYDVVYARQDTADDLKSGVKGMAVLFRNHIEVLLAVLTCTIGGLLAATGVSVGNGPYYFLFSV
AGLTVALLAMIGGIRYRIFHTWNGYSGWFYVLAIINLMSGYFIEYLDNAPILARGS
SEQ ID NO: 65-CBGaS candidate CBGaS 1-Cs.PT4-T
Amino acid sequence
MAGSDQIEGSPHHESDNSIATKILNFGHTCWKLQRPYVVKGMISIACGLFGRELFNNRHLFSWGLMW
KAFFALVPILSFNFFAAIMNQIYDVDIDRINKPDLPLVSGEMSIETAWILSIIVALTGLIVTIKLKSAPLFVFI
YIFGIFAGFAYSVPPIRWKQYPFTNFLITISSHVGLAFTSYSATTSALGLPFVWRPAFSFIIAFMTVMGM
TIAFAKDISDIEGDAKYGVSTVATKLGARNMTFVVSGVLLLNYLVSISIGIIWPQVFKSNIMILSHAILAFC
LIFQTRELALANYASAPSRQFFEFIWLLYYAEYFVYVFI
SEQ ID NO: 66-GPPS candidate isolated from Streptomyces actuosus
Amino acid sequence
MTTEVTSFTGAGPHPAASVRRITDDLLQRVEDKLASFLTAERDRYAAMDERALAAVDALTDLVTSGG
KRVRPTFCITGYLAAGGDAGDPGIVAAAAGLEMLHVSALIHDDILDNSAQRRGKPTIHTLYGDLHDSH
GWRGESRRFGEGIGILIGNLALVYSQELVCQAPPAVLAEWHRLCSEVNIGQCLDVCAAAEFSADPEL
SRLVALIKSGRYTIHRPLVMGANAASRPDLAAAYVEYGEAVGEAFQLRDDLLDAFGDSTETGKPTGLD
FTQHKMTLLLGWAMQRDTHIRTLMTEPGHTPEEVRRRLEDTEVPKDVERHIADLVEQGRAAIADAPID
PQWRQELADMAVRAAYRTN
SEQ ID NO: 67-GPPS candidate IpMSv3
Amino acid sequence
MAFKLAQRLPKSVSSLGSQLSKNAPNQLAAATTSQLINTPGIRHKSRSSAVPSSLSKSMYDHNEEMK
AAMKYMDEIYPEVMGQIEKVPQYEEIKPILVRLREAIDYTVPYGKRFKGVHIVSHFKLLADPKFITPENV
KLSGVLGWCAEIIQAYFCMLDDIMDDSDTRRGKPTWYKLPGIGLNAVTDVCLMEMFTFELLKRYFPKH
PSYADIHEILRNLLFLTHMGQGYDFTFIDPVTRKINFNDFTEENYTKLCRYKIIFSTFHNTLELTSAMANV
YDPKKIKQLDPVLMRIGMMHQSQNDFKDLYRDQGEVLKQAEKSVLGTDIKTGQLTWFAQKALSICND
RQRKIIMDNYGKEDNKNSEAVREVYEELDLKGKFMEFEEESFEWLKKEIPKINNGIPHKVFQDYTYGV
FKRRPE
SEQ ID NO: 68-GPPS candidate SmGPPS_LSUv1
Amino acid sequence
MAFDFKRYMVEKADSVNKALEAVVQMKEPLKIHESMRYSLLAGGKRVRPMLCIAACELVGGEESTA
MPAACAVEMIHTMSLMHDDLPCMDNDDLRRGKPTNHKVFGEDVAVLAGDALLSLAFEHVAVATRGS
APERILRALGQLAKSIGAEGLVAGQVVDICSEGMAEVGLDHLEFIHLHKTAALLQGSVVMGAILGGAKE
EEVERLRKFAKCIGLMFQVVDDILDVTKSSHELGKTAGKDLVADKTTYPKLLGVQKSKEFADDLNREA
QEQLLHFDSHKAAPLIAIANYIAYRNN
SEQ ID NO: 69-GPPS candidate SmGPPS_SSUv1
Amino acid sequence
MAQNHSYWAAIEADIDTYLKKSIAIRSPETVFEPMHHLTFAAPRTAASAICVAACELVGGERSQAIATA
SAIHIMHAAAYAHEHLPLTDRPRPNSKPAIQHKYGPNIELLTGDGMASFGFELLAGSIRSDHPNPERIL
RVIIEISRASGSEGIIDGFYREKEIVDQHSRFDFIEYLCRKKYGEMHACAAASGAILAGGAEEEIQKLRN
FGHYAGTLIGLLHKKIDTPQIQNVIGKLKDLALKELEGFHGKNVELLCSLVADASLCEAELEV
SEQ ID NO: 70-GPPS candidate CrGPPA_LSUv1
Amino acid sequence
MAFDFKAYMIGKANSVNKALEDAVLVREPLKIHESMRYSLLAGGKRVRPMLCIAACELFGGTESVAM
PSACAVEMIHTMSLMHDDLPCMDNDDLRRGKPTNHKVFGEDVAVLAGDALLAFAFEHIATATKGVSS
ERIVRVVGELAKCIGSEGLVAGQVVDVCSEGIADVGLEHLEFIHIHKTAALLEGSVVLGAIVGGANDEQI
SKLRKFARCIGLLFQVVDDILDVTKSSQELGKTAGKDLVADKVTYPKLLGIDKSREFAEKLNREAQEQL
AEFDPEKAAPLIALANYIAYRDN
SEQ ID NO: 71-GPPS candidate CrGPPS_SSUv1
Amino acid sequence
MAMKSNSWANIESDIQTHLKKSIPIRAPEDVFEPMHYLTFAAPRTTAPALCIAACEVVGGDGDQAMAA
AAAIHLVHAAAYAHENLPLTDRRRPKPPIQHKFNSNIELLTGDGIVPYGFELLAKSMDSNNSDRILRVIIE
ITQAAGSKGIIDGQFRELDVIDSEINMGLIEYVCKKKEGELNACGAACGAILGGGSEEEIGKLRKFGLYA
GMIQGLVHGVGKNREEIQELVRKLRYLAMEELKSLKNRKIDTISSLLETDLCSV
SEQ ID NO: 72-Cs.OAC
Amino acid sequence
MAVKHLIVLKFKDEITEAQKEEFFKTYVNLVNIIPAMKDVYWGKDVTQKNKEEGYTHIVEVTFESVETIQ
DYIIHPAHVGFGDVYRSFWEKLLIFDYTPRK
SEQ ID NO: 73-Sc.ACS1
Amino acid sequence
MSPSAVQSSKLEEQSSEIDKLKAKMSQSASTAQQKKEHEYEHLTSVKIVPQRPISDRLQPAIATHYSP
HLDGLQDYQRLHKESIEDPAKFFGSKATQFLNWSKPFDKVFIPDSKTGRPSFQNNAWFLNGQLNACY
NCVDRHALKTPNKKAIIFEGDEPGQGYSITYKELLEEVCQVAQVLTYSMGVRKGDTVAVYMPMVPEAI
ITLLAISRIGAIHSVVFAGFSSNSLRDRINDGDSKVVITTDESNRGGKVIETKRIVDDALRETPGVRHVLV
YRKTNNPSVAFHAPRDLDWATEKKKYKTYYPCTPVDSEDPLFLLYTSGSTGAPKGVQHSTAGYLLGA
LLTMRYTFDTHQEDVFFTAGDIGWITGHTYVVYGPLLYGCATLVFEGTPAYPNYSRYWDIIDEHKVTQ
FYVAPTALRLLKRAGDSYIENHSLKSLRCLGSVGEPIAAEVWEWYSEKIGKNEIPIVDTYWQTESGSHL
VTPLAGGVTPMKPGSASFPFFGIDAVVLDPNTGEELNTSHAEGVLAVKAAWPSFARTIWKNHDRYLD
TYLNPYPGYYFTGDGAAKDKDGYIWILGRVDDVVNVSGHRLSTAEIEAAIIEDPIVAECAVVGFNDDLT
GQAVAAFVVLKNKSNWSTATDDELQDIKKHLVFTVRKDIGPFAAPKLIILVDDLPKTRSGKIMRRILRKIL
AGESDQLGDVSTLSNPGIVRHLIDSVKL
SEQ ID NO: 74-Sc.ACS2
Amino acid sequence
MTIKEHKVVYEAHNVKALKAPQHFYNSQPGKGYVTDMQHYQEMYQQSINEPEKFFDKMAKEYLHW
DAPYTKVQSGSLNNGDVAWFLNGKLNASYNCVDRHAFANPDKPALIYEADDESDNKIITFGELLRKVS
QIAGVLKSWGVKKGDTVAIYLPMIPEAVIAMLAVARIGAIHSVVFAGFSAGSLKDRVVDANSKVVITCD
EGKRGGKTINTKKIVDEGLNGVDLVSRILVFQRTGTEGIPMKAGRDYWWHEEAAKQRTYLPPVSCDA
EDPLFLLYTSGSTGSPKGVVHTTGGYLLGAALTTRYVFDIHPEDVLFTAGDVGWITGHTYALYGPLTL
GTASIIFESTPAYPDYGRYWRIIQRHKATHFYVAPTALRLIKRVGEAEIAKYDTSSLRVLGSVGEPISPD
LWEWYHEKVGNKNCVICDTMWQTESGSHLIAPLAGAVPTKPGSATVPFFGINACIIDPVTGVELEGND
VEGVLAVKSPWPSMARSVWNHHDRYMDTYLKPYPGHYFTGDGAGRDHDGYYWIRGRVDDVVNVS
GHRLSTSEIEASISNHENVSEAAVVGIPDELTGQTVVAYVSLKDGYLQNNATEGDAEHITPDNLRRELI
LQVRGEIGPFASPKTIILVRDLPRTRSGKIMRRVLRKVASNEAEQLGDLTTLANPEVVPAIISAVENQFF
SQKKK
SEQ ID NO: 75-Sc.ALD6
Amino acid sequence
MTKLHFDTAEPVKITLPNGLTYEQPTGLFINNKFMKAQDGKTYPVEDPSTENTVCEVSSATTEDVEYAI
ECADRAFHDTEWATQDPRERGRLLSKLADELESQIDLVSSIEALDNGKTLALARGDVTIAINCLRDAAA
YADKVNGRTINTGDGYMNFTTLEPIGVCGQIIPWNFPIMMLAWKIAPALAMGNVCILKPAAVTPLNALY
FASLCKKVGIPAGVVNIVPGPGRTVGAALTNDPRIRKLAFTGSTEVGKSVAVDSSESNLKKITLELGGK
SAHLVFDDANIKKTLPNLVNGIFKNAGQICSSGSRIYVQEGIYDELLAAFKAYLETEIKVGNPFDKANFQ
GAITNRQQFDTIMNYIDIGKKEGAKILTGGEKVGDKGYFIRPTVFYDVNEDMRIVKEEIFGPVVTVAKFK
TLEEGVEMANSSEFGLGSGIETESLSTGLKVAKMLKAGTVWINTYNDFDSRVPFGGVKQSGYGREM
GEEVYHAYTEVKAVRIKL
SEQ ID NO: 76-Zm.PDC
Amino acid sequence
MSYTVGTYLAERLVQIGLKHHFAVAGDYNLVLLDNLLLNKNMEQVYCCNELNCGFSAEGYARAKGAA
AAVVTYSVGALSAFDAIGGAYAENLPVILISGAPNNNDHAAGHVLHHALGKTDYHYQLEMAKNITAAA
EAIYTPEEAPAKIDHVIKTALREKKPVYLEIACNIASMPCAAPGPASALFNDEASDEASLNAAVEETLKFI
ANRDKVAVLVGSKLRAAGAEEAAVKFADALGGAVATMAAAKSFFPEENPHYIGTSWGEVSYPGVEK
TMKEADAVIALAPVFNDYSTTGWTDIPDPKKLVLAEPRSVVVNGIRFPSVHLKDYLTRLAQKVSKKTG
ALDFFKSLNAGELKKAAPADPSAPLVNAEIARQVEALLTPNTTVIAETGDSWFNAQRMKLPNGARVEY
EMQWGHIGWSVPAAFGYAVGAPERRNILMVGDGSFQLTAQEVAQMVRLKLPVIIFLINNYGYTIEVMI
HDGPYNNIKNWDYAGLMEVFNGNGGYDSGAGKGLKAKTGGELAEAIKVALANTDGPTLIECFIGRED
CTEELVKWGKRVAAANSRKPVNKLL
SEQ ID NO: 77-AACS1
Amino acid sequence
MTDVRFRIIGTGAYVPERIVSNDEVGAPAGVDDDWITRKTGIRQRRWAADDQATSDLATAAGRAALK
AAGITPEQLTVIAVATSTPDRPQPPTAAYVQHHLGATGTAAFDVNAVCSGTVFALSSVAGTLVYRGGY
ALVIGADLYSRILNPADRKTVVLFGDGAGAMVLGPTSTGTGPIVRRVALHTFGGLTDLIRVPAGGSRQ
PLDTDGLDAGLQYFAMDGREVRRFVTEHLPQLIKGFLHEAGVDAADISHFVPHQANGVMLDEVFGEL
HLPRATMHRTVETYGNTGAASIPITMDAAVRAGSFRPGELVLLAGFGGGMAASFALIEW
SEQ ID NO: 78-ACC1
Amino acid sequence
MSEESLFESSPQKMEYEITNYSERHTELPGHFIGLNTVDKLEESPLRDFVKSHGGHTVISKILIANNGIA
AVKEIRSVRKWAYETFGDDRTVQFVAMATPEDLEANAEYIRMADQYIEVPGGTNNNNYANVDLIVDIA
ERADVDAVWAGWGHASENPLLPEKLSQSKRKVIFIGPPGNAMRSLGDKISSTIVAQSAKVPCIPWSG
TGVDTVHVDEKTGLVSVDDDIYQKGCCTSPEDGLQKAKRIGFPVMIKASEGGGGKGIRQVEREEDFI
ALYHQAANEIPGSPIFIMKLAGRARHLEVQLLADQYGTNISLFGRDCSVQRRHQKIIEEAPVTIAKAETF
HEMEKAAVRLGKLVGYVSAGTVEYLYSHDDGKFYFLELNPRLQVEHPTTEMVSGVNLPAAQLQIAMG
IPMHRISDIRTLYGMNPHSASEIDFEFKTQDATKKQRRPIPKGHCTACRITSEDPNDGFKPSGGTLHEL
NFRSSSNVWGYFSVGNNGNIHSFSDSQFGHIFAFGENRQASRKHMVVALKELSIRGDFRTTVEYLIKL
LETEDFEDNTITTGWLDDLITHKMTAEKPDPTLAVICGAATKAFLASEEARHKYIESLQKGQVLSKDLL
QTMFPVDFIHEGKRYKFTVAKSGNDRYTLFINGSKCDIILRQLSDGGLLIAIGGKSHTIYWKEEVAATRL
SVDSMTTLLEVENDPTQLRTPSPGKLVKFLVENGEHIIKGQPYAEIEVMKMQMPLVSQENGIVQLLKQ
PGSTIVAGDIMAIMTLDDPSKVKHALPFEGMLPDFGSPVIEGTKPAYKFKSLVSTLENILKGYDNQVIM
NASLQQLIEVLRNPKLPYSEWKLHISALHSRLPAKLDEQMEELVARSLRRGAVFPARQLSKLIDMAVK
NPEYNPDKLLGAVVEPLADIAHKYSNGLEAHEHSIFVHFLEEYYEVEKLFNGPNVREENIILKLRDENP
KDLDKVALTVLSHSKVSAKNNLILAILKHYQPLCKLSSKVSAIFSTPLQHIVELESKATAKVALQAREILIQ
GALPSVKERTEQIEHILKSSVVKVAYGSSNPKRSEPDLNILKDLIDSNYVVFDVLLQFLTHQDPVVTAA
AAQVYIRRAYRAYTIGDIRVHEGVTVPIVEWKFQLPSAAFSTFPTVKSKMGMNRAVSVSDLSYVANSQ
SSPLREGILMAVDHLDDVDEILSQSLEVIPRHQSSSNGPAPDRSGSSASLSNVANVCVASTEGFESEE
EILVRLREILDLNKQELINASIRRITFMFGFKDGSYPKYYTFNGPNYNENETIRHIEPALAFQLELGRLSN
FNIKPIFTDNRNIHVYEAVSKTSPLDKRFFTRGIIRTGHIRDDISIQEYLTSEANRLMSDILDNLEVTDTSN
SDLNHIFINFIAVFDISPEDVEAAFGGFLERFGKRLLRLRVSSAEIRIIIKDPQTGAPVPLRALINNVSGYVI
KTEMYTEVKNAKGEWVFKSLGKPGSMHLRPIATPYPVKEWLQPKRYKAHLMGTTYVYDFPELFRQA
SSSQWKNFSADVKLTDDFFISNELIEDENGELTEVEREPGANAIGMVAFKITVKTPEYPRGRQFVVVA
NDITFKIGSFGPQEDEFFNKVTEYARKRGIPRIYLAANSGARIGMAEEIVPLFQVAWNDAANPDKGFQ
YLYLTSEGMETLKKFDKENSVLTERTVINGEERFVIKTIIGSEDGLGVECLRGSGLIAGATSRAYHDIFTI
TLVTCRSVGIGAYLVRLGQRAIQVEGQPIILTGAPAINKMLGREVYTSNLQLGGTQIMYNNGVSHLTAV
DDLAGVEKIVEWMSYVPAKRNMPVPILETKDTWDRPVDFTPTNDETYDVRWMIEGRETESGFEYGL
FDKGSFFETLSGWAKGVVVGRARLGGIPLGVIGVETRTVENLIPADPANPNSAETLIQEPGQVWHPN
SAFKTAQAINDFNNGEQLPMMILANWRGFSGGQRDMFNEVLKYGSFIVDALVDYKQPIIIYIPPTGELR
GGSWVVVDPTINADQMEMYADVNARAGVLEPQGMVGIKFRREKLLDTMNRLDDKYRELRSQLSNKS
LAPEVHQQISKQLADRERELLPIYGQISLQFADLHDRSSRMVAKGVISKELEWTEARRFFFWRLRRRL
NEEYLIKRLSHQVGEASRLEKIARIRSWYPASVDHEDDRQVATWIEENYKTLDDKLKGLKLESFAQDL
AKKIRSDHDNAIDGLSEVIKMLSTDDKEKLLKTLK
SEQ ID NO: 79-pGAL1
Nucleic acid sequence
TGGAACTTTCAGTAATACGCTTAACTGCTCATTGCTATATTGAAGTACGGATTAGAAGCCGCCGA
GCGGGCGACAGCCCTCCGACGGAAGACTCTCCTCCGTGCGTCCTGGTCTTCACCGGTCGCGTT
CCTGAAACGCAGATGTGCCTCGCGCCGCACTGCTCCGAACAATAAAGATTCTACAATACTAGCTT
TTATGGTTATGAAGAGGAAAAATTGGCAGTAACCTGGCCCCACAAACCTTCAAATCAACGAATCA
AATTAACAACCATAGGATAATAATGCGATTAGTTTTTTAGCCTTATTTCTGGGGTAATTAATCAGC
GAAGCGATGATTTTTGATCTATTAACAGATATATAAATGCAAAAGCTGCATAACCACTTTAACTAAT
ACTTTCAACATTTTCGGTTTGTATTACTTCTTATTCAAATGTCATAAAAGTATCAACAAAAAATTGTT
AATATACCTCTATACTTTAACGTCAAGGAGAAAAAACTATA
SEQ ID NO: 80-pGAL10
Nucleic acid sequence
CATCGCTTCGCTGATTAATTACCCCAGAAATAAGGCTAAAAAACTAATCGCATTATTATCCTATGG
TTGTTAATTTGATTCGTTGATTTGAAGGTTTGTGGGGCCAGGTTACTGCCAATTTTTCCTCTTCAT
AACCATAAAAGCTAGTATTGTAGAATCTTTATTGTTCGGAGCAGTGCGGCGCGAGGCACATCTGC
GTTTCAGGAACGCGACCGGTGAAGACCAGGACGCACGGAGGAGAGTCTTCCGTCGGAGGGCTG
TCGCCCGCTCGGCGGCTTCTAATCCGTACTTCAATATAGCAATGAGCAGTTAAGCGTATTACTGA
AAGTTCCAAAGAGAAGGTTTTTTTAGGCTAAGATAATGGGGCTCTTTACATTTCCACAACATATAA
GTAAGATTAGATATGGATATGTATATGGTGGTATTGCCATGTAATATGATTATTAAACTTCTTTGCG
TCCATCCAAAAAAAAAGTAAGAATTTTTGAAAATTCAATATAA
SEQ ID NO: 81-pGAL2
Nucleic acid sequence
GGCTTAAGTAGGTTGCAATTTCTTTTTCTATTAGTAGCTAAAAATGGGTCACGTGATCTATATTCG
AAAGGGGCGGTTGCCTCAGGAAGGCACCGGCGGTCTTTCGTCCGTGCGGAGATATCTGCGCCG
TTCAGGGGTCCATGTGCCTTGGACGATATTAAGGCAGAAGGCAGTATCGGGGCGGATCACTCCG
AACCGAGATTAGTTAAGCCCTTCCCATCTCAAGATGGGGAGCAAATGGCATTATACTCCTGCTAG
AAAGTTAACTGTGCACATATTCTTAAATTATACAATGTTCTGGAGAGCTATTGTTTAAAAAACAAAC
ATTTCGCAGGCTAAAATGTGGAGATAGGATTAGTTTTGTAGACATATATAAACAATCAGTAATTGG
ATTGAAAATTTGGTGTTGTGAATTGCTCTTCATTATGCACCTTATTCAATTATCATCAAGAATAGCA
ATAGTTAAGTAAACACAAGATTAACATAATAAAAAAAATAATTCTTTCATA
SEQ ID NO: 82-pGAL3
Nucleic acid sequence
TTTTACTATTATCTTCTACGCTGACAGTAATATCAAACAGTGACACATATTAAACACAGTGGTTTCT
TTGCATAAACACCATCAGCCTCAAGTCGTCAAGTAAAGATTTCGTGTTCATGCAGATAGATAACAA
TCTATATGTTGATAATTAGCGTTGCCTCATCAATGCGAGATCCGTTTAACCGGACCCTAGTGCAC
TTACCCCACGTTCGGTCCACTGTGTGCCGAACATGCTCCTTCACTATTTTAACATGTGGAATTCTT
GAAAGAATGAAATCGCCATGCCAAGCCATCACACGGTCTTTTATGCAATTGATTGACCGCCTGCA
ACACATAGGCAGTAAAATTTTTACTGAAACGTATATAATCATCATAAGCGACAAGTGAGGCAACAC
CTTTGTTACCACATTGACAACCCCAGGTATTCATACTTCCTATTAGCGGAATCAGGAGTGCAAAAA
GAGAAAATAAAAGTAAAAAGGTAGGGCAACACATAGT
SEQ ID NO: 83-pGAL7
Nucleic acid sequence
GGACGGTAGCAACAAGAATATAGCACGAGCCGCGAAGTTCATTTCGTTACTTTTGATATCGCTCA
CAACTATTGCGAAGCGCTTCAGTGAAAAAATCATAAGGAAAAGTTGTAAATATTATTGGTAGTATT
CGTTTGGTAAAGTAGAGGGGGTAATTTTTCCCCTTTATTTTGTTCATACATTCTTAAATTGCTTTGC
CTCTCCTTTTGGAAAGCTATACTTCGGAGCACTGTTGAGCGAAGGCTCATTAGATATATTTTCTGT
CATTTTCCTTAACCCAAAAATAAGGGAAAGGGTCCAAAAAGCGCTCGGACAACTGTTGACCGTGA
TCCGAAGGACTGGCTATACAGTGTTCACAAAATAGCCAAGCTGAAAATAATGTGTAGCTATGTTC
AGTTAGTTTGGCTAGCAAAGATATAAAAGCAGGTCGGAAATATTTATGGGCATTATTATGCAGAG
CATCAACATGATAAAAAAAAACAGTTGAATATTCCCTCAAAA
SEQ ID NO: 84-pGAL4
Nucleic acid sequence
GCGACACAGAGATGACAGACGGTGGCGCAGGATCCGGTTTAAACGAGGATCCCTTAAGTTTAAA
CAACAACAGCAAGCAGGTGTGCAAGACACTAGAGACTCCTAACATGATGTATGCCAATAAAACAC
AAGAGATAAACAACATTGCATGGAGGCCCCAGAGGGGCGATTGGTTTGGGTGCGTGAGCGGCA
AGAAGTTTCAAAACGTCCGCGTCCTTTGAGACAGCATTCGCCCAGTATTTTTTTTATTCTACAAAC
CTTCTATAATTTCAAAGTATTTACATAATTCTGTATCAGTTTAATCACCATAATATCGTTTTCTTTGT
TTAGTGCAATTAATTTTTCCTATTGTTACTTCGGGCCTTTTTCTGTTTTATGAGCTATTTTTTCCGTC
ATCCTTCCCCAGATTTTCAGCTTCATCTCCAGATTGTGTCTACGTAATGCACGCCATCATTTTAAG
AGAGGACAGAGAAGCAAGCCTCCTGAAAG
SEQ ID NO: 85-pMAL1
Nucleic acid sequence
GATGATGGACACTAGTGTGTCGAGAATGTATCAACTATATATAGTCCTAATGCCACACAAATATGA
AGTGGGGGAAGCCCATTCTTAATCCGGCTCAATTTTGGTGCGTGATCGCGGCCTATGTTTGCTTC
CAGAAAAAGCTTAGAATAATATTTCTCACCTTTGATGGAATGCTCGCGAGTGCTCGTTTTGATTAC
CCCATATGCATTGTTGCAGCATGCAAGCACTATTGCAAGCCACGCATGGAAGAAATTTGCAAACA
CCTATAGCCCCGCGTTGTTGAGGAGGTGGACTTGGTGTAGGACCATAAAGCTGTGCACTACTAT
GGTGAGCTCTGTCGTCTGGTGACCTTCTATCTCAGGCACATCCTCGTTTTTGTGCATGAGGTTCG
AGTCACGCCCACGGCCTATTAATCCGCGAAATAAATGCGAAATCTAAATTATGACGCAAGGCTGA
GAGATTCTGACACGCCGCATTTGCGGGGCAGTAATTATCGGGCAGTTTTCCGGGGTTCGGGATG
GGGTTTGGAGAGAAAGTTCAACACAGACCAAAACAGCTTGGGACCACTTGGATGGAGGTCCCCG
CAGAAGAGCTCTGGCGCGTTGGACAAACATTGACAATCCACGGCAAAATTGTCTACAGTTCCGT
GTATGCGGATAGGGATATCTTCGGGAGTATCGCAATAGGATACAGGCACTGTGCAGATTACGCG
ACATGATAGCTTTGTATGTTCTACAGACTCTGCCGTAGCAGTCTAGATATAATATCGGAGTTTTGT
AGCGTCGTAAGGAAAACTTGGGTTACACAGGTTTCTTGAGAGCCCTTTGACGTTGATTGCTCTGG
CTTCCATCCAGGCCCTCATGTGGTTCAGGTGCCTCCGCAGTGGCTGGCAAGCGTGGGGGTCAA
TTACGTCACTTCTATTCATGTACCCCAGACTCAATTGTTGACAGCAATTTCAGCGAGAATTAAATT
CCACAATCAATTCTCGCTGAAATAATTAGGCCGTGATTTAATTCTCGCTGAAACAGAATCCTGTCT
GGGGTACAGATAACAATCAAGTAACTATTATGGACGTGCATAGGAGGTGGAGTCCATGACGCAA
AGGGAAATATTCATTTTATCCTCGCGAAGTTGGGATGTGTCAAAGCGTCGCGCTCGCTATAGTGA
TGAGAATGTCTTTAGTAAGCTTAAGCCATATAAAGACCTTCCGCCTCCATATTTTTTTTTATCCCTC
TTGACAATATTAATTCCTT
SEQ ID NO: 86-pMAL2
Nucleic acid sequence
AAGGAATTAATATTGTCAAGAGGGATAAAAAAAAATATGGAGGCGGAAGGTCTTTATATGGCTTA
AGCTTACTAAAGACATTCTCATCACTATAGCGAGCGCGACGCTTTGACACATCCCAACTTCGCGA
GGATAAAATGAATATTTCCCTTTGCGTCATGGACTCCACCTCCTATGCACGTCCATAATAGTTACT
TGATTGTTATCTGTACCCCAGACAGGATTCTGTTTCAGCGAGAATTAAATCACGGCCTAATTATTT
CAGCGAGAATTGATTGTGGAATTTAATTCTCGCTGAAATTGCTGTCAACAATTGAGTCTGGGGTA
CATGAATAGAAGTGACGTAATTGACCCCCACGCTTGCCAGCCACTGCGGAGGCACCTGAACCAC
ATGAGGGCCTGGATGGAAGCCAGAGCAATCAACGTCAAAGGGCTCTCAAGAAACCTGTGTAACC
CAAGTTTTCCTTACGACGCTACAAAACTCCGATATTATATCTAGACTGCTACGGCAGAGTCTGTA
GAACATACAAAGCTATCATGTCGCGTAATCTGCACAGTGCCTGTATCCTATTGCGATACTCCCGA
AGATATCCCTATCCGCATACACGGAACTGTAGACAATTTTGCCGTGGATTGTCAATGTTTGTCCA
ACGCGCCAGAGCTCTTCTGCGGGGACCTCCATCCAAGTGGTCCCAAGCTGTTTTGGTCTGTGTT
GAACTTTCTCTCCAAACCCCATCCCGAACCCCGGAAAACTGCCCGATAATTACTGCCCCGCAAAT
GCGGCGTGTCAGAATCTCTCAGCCTTGCGTCATAATTTAGATTTCGCATTTATTTCGCGGATTAAT
AGGCCGTGGGCGTGACTCGAACCTCATGCACAAAAACGAGGATGTGCCTGAGATAGAAGGTCA
CCAGACGACAGAGCTCACCATAGTAGTGCACAGCTTTATGGTCCTACACCAAGTCCACCTCCTCA
ACAACGCGGGGCTATAGGTGTTTGCAAATTTCTTCCATGCGTGGCTTGCAATAGTGCTTGCATGC
TGCAACAATGCATATGGGGTAATCAAAACGAGCACTCGCGAGCATTCCATCAAAGGTGAGAAATA
TTATTCTAAGCTTTTTCTGGAAGCAAACATAGGCCGCGATCACGCACCAAAATTGAGCCGGATTA
AGAATGGGCTTCCCCCACTTCATATTTGTGTGGCATTAGGACTATATATAGTTGATACATTCTCGA
CACACTAGTGTCCATCATC
SEQ ID NO: 87-pMAL11
Nucleic acid sequence
GCGCCTCAAGAAAATGATGCTGCAAGAAGAATTGAGGAAGGAACTATTCATCTTACGTTGTTTGT
ATCATCCCACGATCCAAATCATGTTACCTACGTTAGGTACGCTAGGAACTAAAAAAAGAAAAGAA
AAGTATGCGTTATCACTCTTCGAGCCAATTCTTAATTGTGTGGGGTCCGCGAAAATTTCCGGATA
AATCCTGTAAACTTTAACTTAAACCCCGTGTTTAGCGAAATTTTCAACGAAGCGCGCAATAAGGA
GAAATATTATCTAAAAGCGAGAGTTTAAGCGAGTTGCAAGAATCTCTACGGTACAGATGCAACTT
ACTATAGCCAAGGTCTATTCGTATTACTATGGCAGCGAAAGGAGCTTTAAGGTTTTAATTACCCCA
TAGCCATAGATTCTACTCGGTCTATCTATCATGTAACACTCCGTTGATGCGTACTAGAAAATGACA
ACGTACCGGGCTTGAGGGACATACAGAGACAATTACAGTAATCAAGAGTGTACCCAACTTTAACG
AACTCAGTAAAAAATAAGGAATGTCGACATCTTAATTTTTTATATAAAGCGGTTTGGTATTGATTGT
TTGAAGAATTTTCGGGTTGGTGTTTCTTTCTGATGCTACATAGAAGAACATCAAACAACTAAAAAA
ATAGTATAAT
SEQ ID NO: 88-pMAL12
Nucleic acid sequence
ATTATACTATTTTTTTAGTTGTTTGATGTTCTTCTATGTAGCATCAGAAAGAAACACCAACCCGAAA
ATTCTTCAAACAATCAATACCAAACCGCTTTATATAAAAAATTAAGATGTCGACATTCCTTATTTTTT
ACTGAGTTCGTTAAAGTTGGGTACACTCTTGATTACTGTAATTGTCTCTGTATGTCCCTCAAGCCC
GGTACGTTGTCATTTTCTAGTACGCATCAACGGAGTGTTACATGATAGATAGACCGAGTAGAATC
TATGGCTATGGGGTAATTAAAACCTTAAAGCTCCTTTCGCTGCCATAGTAATACGAATAGACCTTG
GCTATAGTAAGTTGCATCTGTACCGTAGAGATTCTTGCAACTCGCTTAAACTCTCGCTTTTAGATA
ATATTTCTCCTTATTGCGCGCTTCGTTGAAAATTTCGCTAAACACGGGGTTTAAGTTAAAGTTTAC
AGGATTTATCCGGAAATTTTCGCGGACCCCACACAATTAAGAATTGGCTCGAAGAGTGATAACGC
ATACTTTTCTTTTCTTTTTTTAGTTCCTAGCGTACCTAACGTAGGTAACATGATTTGGATCGTGGGA
TGATACAAACAACGTAAGATGAATAGTTCCTTCCTCAATTCTTCTTGCAGCATCATTTTCTTGAGG
CGCTCTGGGCAAGGTATAAAAAGTTCCATTAATACGTCTCTAAAAAATTAAATCATCCATCTCTTA
AGCAGTTTTTTTGATAATCTCAAATGTACATCAGTCAAGCGTAACTAAATTACATAA
SEQ ID NO: 89-pMAL31
Nucleic acid sequence
TTATGTATTTTAGTTACGCTTGACTGATGTACATTTGAGATTATCAAAAAAACTGCTTAAGAGATAG
ATGGTTTAATTTTTTAGAGACGTATTAATGGAACTTTTTATACCTTGCCCAGAGCGCCTCAAGAAA
ATGATGCTGAAAGAAGAATTGAGGAAGGAACTACTCATCTTACGTTGTTTGTATCATCCCACGAT
CCAAATCATGTTACCTACGTTAGGTACGCTAGGAACTGAAAAAAGAAAAGAAAAGTATGCGTTAT
CACTCTTCGAGCCAATTCTTAATTGTGTGGGGTCCGCGAAAACTTCCGGATAAATCCTGTAAACT
TAAACTTAAACCCCGTGTTTAGCGAAATTTTCAACGAAGCGCGCAATAAGGAGAAATATTATATAA
AAGCGAGAGTTTAAGCGAGGTTGCAAGAATCTCTACGGTACAGATGCAACTTACTATAGCCAAGG
TCTATTCGTATTGGTATCCAAGCAGTGAAGCTACTCAGGGGAAAACATATTTTCAGAGATCAAAGT
TATGTCAGTCTCTTTTTCATGTGTAACTTAACGTTTGTGCAGGTATCATACCGGCCTCCACATAAT
TTTTGTGGGGAAGACGTTGTTGTAGCAGTCTCCTTATACTCTCCAACAGGTGTTTAAAGACTTCTT
CAGGCCTCATAGTCTACATCTGGAGACAACATTAGATAGAAGTTTCCACAGAGGCAGCTTTCAAT
ATACTTTCGGCTGTGTACATTTCATCCTGAGTGAGCGCATATTGCATAAGTACTCAGTATATAAAG
AGACACAATATACTCCATACTTGTTGTGAGTGGTTTTAGCGTATTCAGTATAACAATAAGAATTAC
ATCCAAGACTATTAATTAACT
SEQ ID NO: 90-pMAL32
Nucleic acid sequence
AGTTAATTAATAGTCTTGGATGTAATTCTTATTGTTATACTGAATACGCTAAAACCACTCACAACAA
GTATGGAGTATATTGTGTCTCTTTATATACTGAGTACTTATGCAATATGCGCTCACTCAGGATGAA
ATGTACACAGCCGAAAGTATATTGAAAGCTGCCTCTGTGGAAACTTCTATCTAATGTTGTCTCCAG
ATGTAGACTATGAGGCCTGAAGAAGTCTTTAAACACCTGTTGGAGAGTATAAGGAGACTGCTACA
ACAACGTCTTCCCCACAAAAATTATGTGGAGGCCGGTATGATACCTGCACAAACGTTAAGTTACA
CATGAAAAAGAGACTGACATAACTTTGATCTCTGAAAATATGTTTTCCCCTGAGTAGCTTCACTGC
TTGGATACCAATACGAATAGACCTTGGCTATAGTAAGTTGCATCTGTACCGTAGAGATTCTTGCAA
CCTCGCTTAAACTCTCGCTTTTATATAATATTTCTCCTTATTGCGCGCTTCGTTGAAAATTTCGCTA
AACACGGGGTTTAAGTTTAAGTTTACAGGATTTATCCGGAAGTTTTCGCGGACCCCACACAATTA
AGAATTGGCTCGAAGAGTGATAACGCATACTTTTCTTTTCTTTTTTCAGTTCCTAGCGTACCTAAC
GTAGGTAACATGATTTGGATCGTGGGATGATACAAACAACGTAAGATGAGTAGTTCCTTCCTCAA
TTCTTCTTTCAGCATCATTTTCTTGAGGCGCTCTGGGCAAGGTATAAAAAGTTCCATTAATACGTC
TCTAAAAAATTAAACCATCTATCTCTTAAGCAGTTTTTTTGATAATCTCAAATGTACATCAGTCAAG
CGTAACTAAAATACATAA

What is claimed is:

1. A method of purifying a cannabinoid from a fermentation composition, the method comprising:
i) culturing a population of host cells that are genetically modified to express one or more enzymes of a cannabinoid biosynthetic pathway in a culture medium and under conditions suitable for the host cells to produce the cannabinoid, thereby producing a fermentation composition;
ii) contacting the fermentation composition with an enzymatic composition comprising a serine protease, and
iii) recovering one or more cannabinoids from the fermentation composition and/or the enzymatic composition.

2. A method of purifying a cannabinoid from a fermentation composition, the method comprising:
i) providing a fermentation composition that has been produced by culturing a population of host cells that are genetically modified to express one or more enzymes of a cannabinoid biosynthetic pathway in a culture medium and under conditions suitable for the host cells to produce the cannabinoid;
ii) contacting the fermentation composition with an enzymatic composition comprising a serine protease, and
iii) recovering one or more cannabinoids from the fermentation composition and/or the enzymatic composition.

3. The method of claim 1 or 2, wherein following the culturing of the population of host cells, the fermentation composition is separated into a supernatant and a pellet by solid-liquid centrifugation.

4. The method of any one of claims 1-3, wherein the fermentation composition is contacted with the enzymatic composition after the fermentation is adjusted to a pH of about 7.

5. The method of any one of claims 1-4, wherein the final concentration of the enzymatic composition is from about 0.5% (w/v) to about 3% (w/v) after contacting the fermentation composition with the enzymatic composition.

6. The method of claim 5, wherein the fermentation composition is contacted with the enzymatic composition at a final concentration of about 1% (w/v).

7. The method of any one of claims 1-6, wherein the fermentation composition is mixed with the enzymatic composition for between 0.5 hours and 2 hours.

8. The method of claim 7, wherein the fermentation composition is mixed with the enzymatic composition for about 60 minutes.

9. The method of claim 7 or 8, wherein the fermentation composition is maintained at 55° C.

10. The method of any one of claims 1-9, wherein the enzymatic composition comprises between 0.003% and 20% serine protease by weight.

11. The method of claim 10, wherein the enzymatic composition comprises between 0.01% and 10% serine protease by weight.

12. The method of claim 11, wherein the enzymatic composition comprises between 0.01% and 5% by serine protease by weight.

13. The method of any one of claims 1-12, wherein the serine protease is a subtilisin.

14. The method of claim 13, wherein the subtilisin is from Bacillus licheniformis.

15. The method of claim 14, wherein the subtilisin is subtilisin Carlsberg.

16. The method of claim 15, wherein the subtilisin has an amino acid sequence that is at least 90% identical to the amino acid sequence of SEQ ID NO: 1.

17. The method of claim 16, wherein the subtilisin has an amino acid sequence that is at least 95% identical to the amino acid sequence of SEQ ID NO: 1.

18. The method of claim 17, wherein the subtilisin has the amino acid sequence of SEQ ID NO: 1.

19. The method of any one of claims 1-18, wherein the serine protease is deactivated by exposure to 300 ppm hypochlorite at a temperature of 85° F. for less than one minute; 3.5 ppm hypochlorite at a temperature of 100° F. for 2 min; a pH below 4 for 30 min at a temperature of 140° F.; or by heating to a temperature of 175° F. for 10 min.

20. The method of any one of claims 1-18, wherein the serine protease is deactivated by liquid/liquid centrifugation at 70° C.

21. The method of any one of claims 1-20, wherein the enzymatic composition comprises an alkylaryl sulfonate salt.

22. The method of claim 21, wherein the alkylaryl sulfonate comprises a linear alkylaryl sulfonate salt.

23. The method of any one of claims 1-22, wherein the enzymatic composition comprises a phosphate salt.

24. The method of any one of claims 1-23, wherein the enzymatic composition comprises a carbonate salt.

25. The method of any one of claims 21-24, wherein the salt is a sodium salt.

26. The method of any one of claims 1-25, wherein the enzymatic composition has a pH of between 8.5 and 11 in a 1% (w/v) solution.

27. The method of claim 26, wherein the enzymatic composition has a pH of about 9.5 in a 1% (w/v) solution.

28. The method of any one of claims 1-27, wherein the fermentation composition undergoes liquid-liquid centrifugation after being contacted with the enzymatic composition.

29. The method of any one of claims 1-28, wherein the fermentation composition is passed through an evaporator after being contacted with the enzymatic composition.

30. The method of claim 29, wherein the fermentation composition is passed through an evaporator more than once.

31. The method of claim 30, wherein the fermentation composition is passed through an evaporator twice.

32. The method of any one of claims 29-31, wherein the walls of the evaporator are heated to a temperature of about 180° C.

33. The method of claim 29-32, wherein the walls of the evaporator are heated to a temperature of about 250° C.

34. The method of any one of claims 29-33, wherein the condenser of the evaporator is heated to a temperature of about 80° C.

35. The method of claim 34, wherein the walls of the evaporator are heated to a temperature of about 180° C. and the condenser of the evaporator is heated to a temperature of 80° C. the first time the fermentation composition is passed through the evaporator, and the walls of the evaporator are heated to a temperature of about 250° C. and the condenser of the evaporator is heated to a temperature of 80° C. the second time the fermentation composition is passed through the evaporator.

36. The method of any one of claims 29-35, wherein the evaporate is a short-path evaporator.

37. The method of any one of claims 29-36, wherein the fermentation composition is heated to a temperature of 180° C. or more for less than 5 minutes.

38. The method of claim 37, wherein the fermentation composition is heated to a temperature of 180° C. or more for less than 1 minute.

39. The method of any one of claims 1-38, wherein the cannabinoid is recovered using crystallization after the fermentation solution is passed through the evaporator.

40. The method of any one of claims 1-39, wherein the recovered cannabinoid has between 50% and 100% purity.

41. The method of claim 40, wherein the recovered cannabinoid has between 70% and 100% purity.

42. The method of any one of claims 1-41, wherein the molar yield of the cannabinoid is between 60% and 100%,

43. The method of claim 42, wherein the molar yield is between 90% and 100%.

44. The method of any one of claims 1-43, wherein the host cells comprise one or more heterologous nucleic acids that each, independently, encode (a) an acyl activating enzyme (AAE), and/or (b) a tetraketide synthase (TKS), and/or (c) a cannabigerolic acid synthase (CBGaS), and/or (d) a geranyl pyrophosphate (GPP) synthase.

45. The method of claim 44, wherein the host cells comprise heterologous nucleic acids that independently encode (a) an AAE, (b) a TKS, (c) a CBGaS, and (d) a GPP synthase.

46. The method of claim 44 or 45, wherein the host cell comprises a heterologous nucleic acid that encodes an AAE having an amino acid sequence that is at least 90% identical to the amino acid sequence of any one of SEQ ID NO: 2-25.

47. The method of claim 46, wherein the AAE has an amino acid sequence that is at least 95% identical to the amino acid sequence of any one of SEQ ID NO:2-25.

48. The method of claim 47, wherein the AAE has the amino acid sequence of any one of SEQ ID NO: 2-25.

49. The method of claim 44 or 45, wherein the host cell comprises a heterologous nucleic acid that encodes an AAE having an amino acid sequence that is at least 90% identical to the amino acid sequence of any one of SEQ ID NO: 2-14.

50. The method of claim 49, wherein the AAE has an amino acid sequence that is at least 95% identical to the amino acid sequence of any one of SEQ ID NO: 2-14.

51. The method of claim 50, wherein the AAE has the amino acid sequence of any one of SEQ ID NO: 2-14.

52. The method of any one of claims 44-51, wherein the host cell comprises a heterologous nucleic acid that encodes a TKS having an amino acid sequence that is at least 90% identical to the amino acid sequence of any one of SEQ ID NO: 26-60.

53. The method of claim 52, wherein the TKS has an amino acid sequence that is at least 95% identical to the amino acid sequence of any one of SEQ ID NO: 26-60.

54. The method of claim 53, wherein the TKS has the amino acid sequence of any one of SEQ ID NO: 26-60.

55. The method of any one of claims 44-51, wherein the host cell comprises a heterologous nucleic acid that encodes a TKS having an amino acid sequence that is at least 90% identical to the amino acid sequence of any one of SEQ ID NO: 26-29.

56. The method of claim 55, wherein the TKS has an amino acid sequence that is at least 95% identical to the amino acid sequence of any one of SEQ ID NO: 26-29.

57. The method of claim 56, wherein the TKS has the amino acid sequence of any one of SEQ ID NO: 26-29.

58. The method of any one of claims 44-51, wherein the host cell comprises a heterologous nucleic acid that encodes a TKS having an amino acid sequence that is at least 90% identical to the amino acid sequence of SEQ ID NO: 26.

59. The method of claim 58, wherein the TKS has an amino acid sequence that is at least 95% identical to the amino acid sequence of SEQ ID NO: 26.

60. The method of claim 59, wherein the TKS has the amino acid sequence of SEQ ID NO: 26.

61. The method of any one of claims 44-60, wherein the host cell comprises a heterologous nucleic acid that encodes a CBGaS having an amino acid sequence that is at least 90% identical to the amino acid sequence of any one of SEQ ID NO: 61-65.

62. The method of claim 61, wherein the CBGaS has an amino acid sequence that is at least 95% identical to the amino acid sequence of any one of SEQ ID NO: 61-65.

63. The method of claim 62, wherein the CBGaS has the amino acid sequence of any one of SEQ ID NO: 61-65.

64. The method of any one of claims 44-63, wherein the host cell comprises a heterologous nucleic acid that encodes a GPP synthase having an amino acid sequence that is at least 90% identical to the amino acid sequence of any one of SEQ ID NO: 66-71.

65. The method of claim 64, wherein the GPP synthase has an amino acid sequence that is at least 95% identical to the amino acid sequence of any one of SEQ ID NO: 66-71.

66. The method of claim 65, wherein the GPP synthase has the amino acid sequence of any one of SEQ ID NO: 66-71.

67. The method of any one of claims 44-66, wherein the host cell comprises a heterologous nucleic acid that encodes a GPP synthase having an amino acid sequence that is at least 90% identical to the amino acid sequence of SEQ ID NO: 66.

68. The method of claim 67, wherein the GPP synthase has an amino acid sequence that is at least 95% identical to the amino acid sequence of SEQ ID NO: 66.

69. The method of claim 68, wherein the GPP synthase has the amino acid sequence of SEQ ID NO: 66.

70. The method of any one of claims 44-69, wherein the host cell comprises heterologous nucleic acids that independently encode
(a) an AAE having the amino acid sequence of any one of SEQ ID NO: 2-25,
(b) a TKS having the amino acid sequence of any one of SEQ ID NO: 26-60,
(c) a CBGaS having the amino acid sequences of any one of SEQ ID NO: 61-65, and
(d) a GPP synthase having the amino acid sequence of any one of SEQ ID NO: 66-71.

71. The method of any one of claims 1-70, wherein the host cell further comprises one or more heterologous nucleic acids that each, independently, encode an enzyme of the mevalonate biosynthetic pathway, wherein the enzyme is selected from an acetyl-CoA thiolase, an HMG-CoA synthase, an HMG-CoA reductase, a mevalonate kinase, a phosphomevalonate kinase, a mevalonate pyrophosphate decarboxylase, and an IPP:DMAPP isomerase.

72. The method of claim 71, wherein the host cell comprises heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

73. The method of any one of claims 1-72, the host cell further comprises a heterologous nucleic acid that encodes an olivetolic acid cyclase (OAC).

74. The method of claim 73, wherein the OAC has an amino acid sequence that is at least 90% identical to the amino acid sequence of SEQ ID NO: 72.

75. The method of claim 74, wherein the OAC has an amino acid sequence that is at least 95% identical to the amino acid sequence of SEQ ID NO: 72.

76. The method of claim 75, wherein the OAC has the amino acid sequence of SEQ ID NO: 72.

77. The method of any one of claims 1-76, wherein the host cell further comprises one or more heterologous nucleic acids that each, independently, encode an acetyl-CoA synthase, and/or an aldehyde dehydrogenase, and/or a pyruvate decarboxylase.

78. The method of claim 77, wherein the acetyl-CoA synthase has an amino acid sequence that is at least 90% identical to the amino acid sequence of SEQ ID NO: 73.

79. The method of claim 78, wherein the acetyl-CoA synthase has an amino acid sequence that is at least 95% identical to the amino acid sequence of SEQ ID NO: 73.

80. The method of claim 79, wherein the acetyl-CoA synthase has the amino acid sequence of SEQ ID NO: 73.

81. The method of claim 77, wherein the acetyl-CoA synthase has an amino acid sequence that is at least 90% identical to the amino acid sequence of SEQ ID NO: 74.

82. The method of claim 81, wherein the acetyl-CoA synthase has an amino acid sequence that is at least 95% identical to the amino acid sequence of SEQ ID NO: 74.

83. The method of claim 82, wherein the acetyl-CoA synthase has the amino acid sequence of SEQ ID NO: 74.

84. The method of any one of claims 77-83, wherein the aldehyde dehydrogenase has an amino acid sequence that is at least 90% identical to the amino acid sequence of SEQ ID NO: 75.

85. The method of claim 84, wherein the aldehyde dehydrogenase has an amino acid sequence that is at least 95% identical to the amino acid sequence of SEQ ID NO: 75.

86. The method of claim 85, wherein the aldehyde dehydrogenase synthase has the amino acid sequence of SEQ ID NO: 75.

87. The method of any one of claims 77-86, wherein the pyruvate decarboxylase has an amino acid sequence that is at least 90% identical to the amino acid sequence of SEQ ID NO: 76.

88. The method of claim 87, wherein the pyruvate decarboxylase has an amino acid sequence that is at least 95% identical to the amino acid sequence of SEQ ID NO: 76.

89. The method of claim 88, wherein the pyruvate decarboxylase has the amino acid sequence of SEQ ID NO: 76.

90. The method of any one of claims 44-89, wherein expression of the one or more heterologous nucleic acids are regulated by an exogenous agent.

91. The method of claim 90, wherein the exogenous agent comprises a regulator of gene expression.

92. The method of claim 90 or 91, wherein the exogenous agent decreases production of the cannabinoid.

93. The method of claim 92, wherein the exogenous agent is maltose.

94. The method of claim 90 or 91, wherein the exogenous agent increases production of the cannabinoid.

95. The method of claim 94, wherein the exogenous agent is galactose.

96. The method of claim 95, wherein the exogenous agent is galactose and expression of one or more heterologous nucleic acids encoding the AAE, TKS, and CBGaS enzymes is under the control of a GAL promoter.

97. The method of any one of claims 44-96, wherein expression of one or more heterologous nucleic acids encoding the AAE, TKS, and CBGaS enzymes is under the control of a galactose-responsive promoter, a maltose-responsive promoter, or a combination of both.

98. The method of any one of claims 1-97, further comprising culturing the host cell with a precursor required to make the cannabinoid.

99. The method of claim 98, wherein the precursor required to make the cannabinoid is hexanoate.

100. The method of any one of claims 1-99, wherein the cannabinoid is cannabidiolic acid (CBDA), cannabidiol (CBD) or an acid form thereof, cannabigerolic acid (CBGA), cannabigerol (CBG) or an acid form thereof, tetrahydrocannabinol (THC) or an acid form thereof, or tetrahydrocannabinolic acid (THCa).

101. The method of any one of claims 1-100, wherein the host cell is a yeast cell or yeast strain.

102. The method of claim 101, wherein the yeast cell is S. cerevisiae.

103. A method of decarboxylating a cannabinoid, the method comprising contacting an enzymatic composition comprising a serine protease with a fermentation composition, wherein the fermentation composition:
(i) comprises a population of host cells that are genetically modified to express one or more enzymes of a cannabinoid biosynthetic pathway; and
(ii) has been cultured in a culture medium and under conditions suitable for the host cells to produce the cannabinoid.

104. The method of claim 103, wherein the fermentation composition is separated into a supernatant and a pellet by solid-liquid centrifugation.

105. The method of claim 103 or 104, wherein the fermentation composition is contacted with the enzymatic composition after the fermentation is adjusted to a pH of about 7.

106. The method of any one of claims 103-105, wherein the final concentration of the enzymatic composition is from about 0.5% (w/v) to about 1% (w/v) after contacting the fermentation composition with the enzymatic composition.

107. The method of claim 106, wherein the fermentation composition is contacted with the enzymatic composition at a final concentration of about 1% (w/v).

108. The method of any one of claims 103-107, wherein the fermentation composition is mixed with the enzymatic composition for between 0.5 hours and 2 hours.

109. The method of claim 108, wherein the fermentation composition is mixed with the enzymatic composition for about 60 minutes.

110. The method of claim 108 or 109, wherein the fermentation composition is maintained at a temperature of 55° C.

111. The method of any one of claims 103-110, wherein the enzymatic composition comprises between 0.003% and 20% serine protease by weight.

112. The method of claim 111, wherein the enzymatic composition comprises between 0.01% and 10% serine protease by weight.

113. The method of claim 112, wherein the enzymatic composition comprises between 0.01% and 5% by serine protease by weight.

114. The method of any one of claims 103-113, wherein the serine protease is a subtilisin.

115. The method of claim 114, wherein the subtilisin is from Bacillus licheniformis.

116. The method of claim 115, wherein the subtilisin is subtilisin Carlsberg.

117. The method of claim 116, wherein the subtilisin has an amino acid sequence that is at least 90% identical to the amino acid sequence of SEQ ID NO: 1.

118. The method of claim 117, wherein the subtilisin has an amino acid sequence that is at least 95% identical to the amino acid sequence of SEQ ID NO: 1.

119. The method of claim 118, wherein the subtilisin has the amino acid sequence of SEQ ID NO: 1.

120. The method of any one of claims 103-119, wherein the enzymatic composition comprises an alkylaryl sulfonate salt.

121. The method of claim 120, wherein the alkylaryl sulfonate comprises a linear alkylaryl sulfonate salt.

122. The method of any one of claims 103-121, wherein the enzymatic composition comprises a phosphate salt.

123. The method of any one of claims 103-122, wherein the enzymatic composition comprises a carbonate salt.

124. The method of any one of claims 103-123, wherein the enzymatic composition has a pH of between 8.5 and 11 in a 1% (w/v) solution.

125. The method of claim 124, wherein the enzymatic composition has a pH of about 9.5 in a 1% (w/v) solution.

126. The method of any one of claims 103-125, wherein the fermentation composition undergoes liquid-liquid centrifugation after being contacted with the enzymatic composition.

127. The method of any one of claims 103-126, wherein the fermentation composition is passed through an evaporator after being contacted with the enzymatic composition.

128. The method of claim 127, wherein the fermentation composition is passed through an evaporator more than once.

129. The method of claim 128, wherein the fermentation composition is passed through an evaporator twice.

130. The method of any one of claims 127-129, wherein the walls of the evaporator are heated to a temperature of about 180° C.

131. The method of claim 127-130, wherein the walls of the evaporator are heated to a temperature of about 250° C.

132. The method of any one of claims 127-131, wherein the condenser of the evaporator is heated to a temperature of 80° C.

133. The method of claim 132, wherein the walls of the evaporator are heated to a temperature of about 180° C. and the condenser of the evaporator is heated to a temperature of 80° C. the first time the fermentation composition is passed through the evaporator, and the walls of the evaporator are heated to a temperature of about 250° C. and the condenser of the evaporator is heated to a temperature of 80° C. the second time the fermentation composition is passed through the evaporator.

134. The method of any one of claims 127-133, wherein the evaporate is a short-path evaporator.

135. The method of any one of claims 127-134, wherein the fermentation composition is heated to a temperature of 180° C. or more for less than 5 minutes.

136. The method of claim 135, wherein the fermentation composition is heated to a temperature of 180° C. or more for less than 1 minute.

137. The method of any one of claims 103-136, wherein the host cells comprise one or more heterologous nucleic acids that each, independently, encode (a) an AAE, and/or (b) a TKS, and/or a (c) CBGaS, and/or (d) a GPP synthase.

138. The method of claim 137, wherein the host cells comprise heterologous nucleic acids that independently encode (a) an AAE, (b) a TKS, (c) a CBGaS, and (d) a GPP synthase.

139. The method of claim 137 or 138, wherein the AAE has an amino acid sequence that is at least 90% identical to the amino acid sequence any one of SEQ ID NO: 2-25.

140. The method of claim 139, wherein the AAE has an amino acid sequence that is at least 95% identical to the amino acid sequence of any one of SEQ ID NO: 2-25.

141. The method of claim 140, wherein the AAE has the amino acid sequence of any one of SEQ ID NO: 2-25.

142. The method of claim 137 or 138, wherein the AAE has an amino acid sequence that is at least 90% identical to the amino acid sequence any one of SEQ ID NO: 2-14.

143. The method of claim 142, wherein the AAE has an amino acid sequence that is at least 95% identical to the amino acid sequence of any one of SEQ ID NO: 2-14.

144. The method of claim 143, wherein the AAE has the amino acid sequence of any one of SEQ ID NO: 2-14.

145. The method of claim 137 or 138, wherein the AAE has an amino acid sequence that is at least 90% identical to the amino acid sequence any one of SEQ ID NO: 2-6.

146. The method of claim 145, wherein the AAE has an amino acid sequence that is at least 95% identical to the amino acid sequence of any one of SEQ ID NO: 2-6.

147. The method of claim 146, wherein the AAE has the amino acid sequence of any one of SEQ ID NO: 2-6.

148. The method of any one of claims 137-147, wherein the TKS has an amino acid sequence that is at least 90% identical to the amino acid sequence of any one of SEQ ID NO: 26-60.

149. The method of claim 148, wherein the TKS has an amino acid sequence that is at least 95% identical to the amino acid sequence of any one of SEQ ID NO: 26-60.

150. The method of claim 149, wherein the TKS has the amino acid sequence of any one of SEQ ID NO: 26-60.

151. The method of any one of claims 137-147, wherein the TKS has an amino acid sequence that is at least 90% identical to the amino acid sequence of any one of SEQ ID NO: 26-29.

152. The method of claim 151, wherein the TKS has an amino acid sequence that is at least 95% identical to the amino acid sequence of any one of SEQ ID NO: 26-29.

153. The method of claim 152, wherein the TKS has the amino acid sequence of any one of SEQ ID NO: 26-29.

154. The method of any one of claims 137-147, wherein the TKS has an amino acid sequence that is at least 90% identical to the amino acid sequence of SEQ ID NO: 26.

155. The method of claim 154, wherein the TKS has an amino acid sequence that is at least 95% identical to the amino acid sequence of SEQ ID NO: 26.

156. The method of claim 155, wherein the TKS has the amino acid sequence of SEQ ID NO: 26.

157. The method of any one of claims 137-156, wherein the CBGaS has an amino acid sequence that is at least 90% identical to the amino acid sequence of any one of SEQ ID NO: 61-65.

158. The method of claim 157, wherein the CBGaS has an amino acid sequence that is at least 95% identical to the amino acid sequence of any one of SEQ ID NO: 61-65.

159. The method of claim 158, wherein the CBGaS has the amino acid sequence of any one of SEQ ID NO: 61-65.

160. The method of any one of claims 137-159, wherein the GPP synthase has an amino acid sequence that is at least 90% identical to the amino acid sequence any one of SEQ ID NO: 66-71.

161. The method of claim 160, wherein the GPP synthase has an amino acid sequence that is at least 95% identical to the amino acid sequence of any one of SEQ ID NO: 66-71.

162. The method of claim 161, wherein the GPP synthase has the amino acid sequence of any one of SEQ ID NO: 66-71.

163. The method of any one of claims 137-162, wherein the host cell comprises heterologous nucleic acids that independently encode
(a) an AAE having the amino acid sequence of any one of SEQ ID NO: 2-25,
(b) a TKS having the amino acid sequence of any one of SEQ ID NO: 26-60,
(c) a CBGaS having the amino acid sequences of any one of SEQ ID NO: 61-65, and
(d) a GPP synthase having the amino acid sequence of any one of 66-71.

164. The method of any one of claims 103-163, wherein the host cell further comprises one or more heterologous nucleic acids that each, independently, encode an enzyme of the mevalonate biosynthetic pathway, wherein the enzyme is selected from an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

165. The method of claim 164, wherein the host cell comprises heterologous nucleic acids that independently encode an acetyl-CoA thiolase, an HMG-COA synthase, an HMG-CoA reductase, a mevalonate kinase, a phosphomevalonate kinase, a mevalonate pyrophosphate decarboxylase, and an IPP:DMAPP isomerase.

166. The method of any one of claims 103-165, the host cell further comprises a heterologous nucleic acid that encodes an olivetolic acid cyclase (OAC).

167. The method of claim 166, wherein the OAC has an amino acid sequence that is at least 90% identical to the amino acid sequence of SEQ ID NO: 72.

168. The method of claim 167, wherein the OAC has an amino acid sequence that is at least 95% identical to the amino acid sequence of SEQ ID NO: 72.

169. The method of claim 168, wherein the OAC has the amino acid sequence of SEQ ID NO: 72.

170. The method of any one of claims 103-169, wherein the host cell further comprises one or more heterologous nucleic acids that each, independently, encode an acetyl-CoA synthase, and/or an aldehyde dehydrogenase, and/or a pyruvate decarboxylase.

171. The method of claim 170, wherein the acetyl-CoA synthase has an amino acid sequence that is at least 90% identical to the amino acid sequence of SEQ ID NO: 73.

172. The method of claim 171, wherein the acetyl-CoA synthase has an amino acid sequence that is at least 95% identical to the amino acid sequence of SEQ ID NO: 73.

173. The method of claim 172, wherein the acetyl-CoA synthase has the amino acid sequence of SEQ ID NO: 73.

174. The method of claim 170, wherein the acetyl-CoA synthase has an amino acid sequence that is at least 90% identical to the amino acid sequence of SEQ ID NO: 74.

175. The method of claim 174, wherein the acetyl-CoA synthase has an amino acid sequence that is at least 95% identical to the amino acid sequence of SEQ ID NO: 74.

176. The method of claim 175, wherein the acetyl-CoA synthase has the amino acid sequence of SEQ ID NO: 74.

177. The method of any one of claims 170-176, wherein the aldehyde dehydrogenase has an amino acid sequence that is at least 90% identical to the amino acid sequence of SEQ ID NO: 75.

178. The method of claim 177, wherein the aldehyde dehydrogenase has an amino acid sequence that is at least 95% identical to the amino acid sequence of SEQ ID NO: 75.

179. The method of claim 178, wherein the aldehyde dehydrogenase synthase has the amino acid sequence of SEQ ID NO: 75.

180. The method of any one of claims 170-179, wherein the pyruvate decarboxylase has an amino acid sequence that is at least 90% identical to the amino acid sequence of SEQ ID NO: 76.

181. The method of claim 180, wherein the pyruvate decarboxylase has an amino acid sequence that is at least 95% identical to the amino acid sequence of SEQ ID NO: 76.

182. The method of claim 181, wherein the pyruvate decarboxylase has the amino acid sequence of SEQ ID NO: 76.

183. The method of any one of claims 137-182, wherein expression of the one or more heterologous nucleic acids are regulated by an exogenous agent.

184. The method of claim 183, wherein the exogenous agent comprises a regulator of gene expression.

185. The method of claim 183 or 184, wherein the exogenous agent decreases production of the cannabinoid.

186. The method of claim 185, wherein the exogenous agent is maltose.

187. The method of claim 183 or 184, wherein the exogenous agent increases production of the cannabinoid.

188. The method of claim 187, wherein the exogenous agent is galactose.

189. The method of claim 188, wherein the exogenous agent is galactose and expression of one or more heterologous nucleic acids encoding the AAE, TKS, and CBGaS enzymes is under the control of a GAL promoter.

190. The method of any one of claims 137-189, wherein expression of one or more heterologous nucleic acids encoding the AAE, TKS, and CBGaS enzymes is under the control of a galactose-responsive promoter, a maltose-responsive promoter, or a combination of both.

191. The method of any one of claims 103-190, wherein the culture medium comprises a precursor required to make the cannabinoid.

192. The method of claim 191, wherein the precursor required to make the cannabinoid is hexanoate.

193. The method of any one of claims 103-192, wherein the cannabinoid is CBDA, CBD or an acid form thereof, CBGA, CBG or an acid form thereof, THC or an acid form thereof, or THCa.

194. The method of any one of claims 103-193, wherein the host cell is a yeast cell or yeast strain.

195. The method of claim 194, wherein the yeast cell is S. cerevisiae.

196. A mixture comprising:
(i) a fermentation composition produced by culturing a population of host cells that are genetically modified to express one or more enzymes of a cannabinoid biosynthetic pathway in a culture medium and under conditions suitable for the host cells to produce the cannabinoid; and
(ii) an enzymatic composition comprising a serine protease.

197. The mixture of claim 196, wherein the serine protease is a subtilisin from Bacillus licheniformis.

198. The mixture of claim 196 or 197, wherein the enzymatic composition comprises sodium linear alkylaryl sulfonates, phosphates, and carbonates.

199. The mixture of any one of claims 196-198, wherein the host cells comprise one or more heterologous nucleic acids that each, independently, encode (a) an AAE, and/or (b) a TKS, and/or (c) a CBGaS, and/or (d) a GPP synthase.

200. The mixture of claim 199 wherein the host cell further comprises one or more heterologous nucleic acids that each, independently, encode an enzyme of the mevalonate biosynthetic pathway, wherein the enzyme is selected from an acetyl-CoA thiolase, an HMG-COA synthase, an HMG-COA reductase, a mevalonate kinase, a phosphomevalonate kinase, a mevalonate pyrophosphate decarboxylase, and an IPP:DMAPP isomerase.


SEQUENCE LISTING (90 sequences)

SEQ ID NO: 1 - protein, 379 aa, Bacillus licheniformis
MMRKKSFWLGMLTAFMLVFTMAFSDSASAAQPAKNVEKDYIVGFKSGVKTASVKKDIIKESGGKVDKQFRIINAAKAKLDKEALKEVKNDPDVAYVEEDHVAHALAQTVPYGIPLIKADKVQAQGFKGANVKVAVLDTGIQASHPDLNVVGGASFVAGEAYNTDGNGHGTHVAGTVAALDNTTGVLGVAPSVSLYAVKVLNSSGSGSYSGIVSGIEWATTNGMDVINMSLGGASGSTAMKQAVDNAYAKGVVVVAAAGNSGSSGNTNTIGYPAKYDSVIAVGAVDSNSNRASFSSVGAELEVMAPGAGVYSTYPTNTYATLNGTSMASPHVAGAAALILSKHPNLSASQVRNRLSSTATYLGSSFYYGKGLINVEAAAQ

SEQ ID NO: 2 - protein, 541 aa, Pseudonocardia sp. N23
MTAAQAPDPAGVPLVERTVPRMLARSAALDPDRPFVVTRERTWSHTDAHRIVATLAAAFTDRGIGQGSRVAVMMPTSPRHVWLLLALAHLRAVPVALNPDASGEVLRYFVADSECVLGVVDQERAAAFATAAGPDGPPAIVLPPGADDLGELGSAGPGPLDPGAASFSDTFVVLYTSGSTGMPKATAVTHAQVITCGAVFTDRLGLGPADRLYTCLPLFHINATAYSLSGALVSGASLALGPHFSATTFWDDVADLGATEVNAMGSMVRILQSRPPRPAERAHRVRTMFVAPLPPDAVELSERFGLDFATCYAQTEWLPSSMTRPGEGYGRPGATGPVLPWTEVRIVGDDDRPLPAGQTGEIILRPRDPYTTFQGYLGKPQETVDAWRNLWFHTGDLGDIGPDGWLHYRGRRKDVIRRRGENIPATVVEDLLAGHPDIAEVAAVSVPAHISEEEIFAFVVPGAGAALTTADVEAHAHAVLPRYMVPSYLALVPDLPRTATNKIAKVELTERARAAVEGTGDPADAPTRTSAADRVVVPAAE

SEQ ID NO: 3 - protein, 548 aa, Pseudomonas putida
MMVPTLEHELAPNEANHVPLSPLSFLKRAAQVYPQRDAVIYGARRYSYRQLHERSRALASALERVGVQPGERVAILAPNIPEMLEAHYGVPGAGAVLVCINIRLEGRSIAFILRHCAAKVLICDREFGAVANQALAMLDAPPLLVGIDDDQAERADLAHDLDYEAFLAQGDPARPLSAPQNEWQSIAINYTSGTTGDPKGVVLHHRGAYLNACAGALIFQLGPRSVYLWTLPMFHCNGWSHTWAVTLSGGTHVCLRKVQPDAINAAIAEHAVTHLSAAPVVMSMLIHAEHASAPPVPVSVITGGAAPPSAVIAAMEARGFNITHAYGMTESYGPSTLCLWQPGVDELPLEARAQFMSRQGVAHPLLEEATVLDTDTGRPVPADGLTLGELVVRGNTVMKGYLHNPEATRAALANGWLHTGDLAVLHLDGYVEIKDRAKDIIISGGENISSLEIEEVLYQHPEVVEAAVVARPDSRWGETPHAFVTLRADALASGDDLVRWCRERLAHFKAPRHVSLVDLPKTATGKIQKFVLREWARQQEAQIADAEH

SEQ ID NO: 4 - protein, 549 aa, Streptomyces sp.ADI96-02
MLSTMQDVPLTVTRILQHGMTIHGKSQVTTWTGEPEPHRRTFAEIGARATRLAHALRDELGIDGDQRVATLMWNNAEHVEAYLAVPSMGAVLHTLNLRLPAEQLIWIVNHADDKVVIVNGSLLPLLVPLLPHLPTVEHVVVSGPGDRSALAGVAPRVHEYEELIADRPTTYDWPELDERQAAAMCYTSGTTGDPKGVVYSHRSVYLHSMQVNMTESMGLTDKDTTLVVVPQFHVNAWGLPHATFMAGVNMLMPDRFLQPAPLADMIERERPTHAAAVPTIWQGLLAEVTAHPRDLTSMASVTIGGAACPPSLMEAYDKLGVRLCHAWGMTETSPLGTMANPPAGLSAEEEWPYRVTQGRFPAGVEARLVGPAGDHLPWDGRSAGELEVRGAWIAGAYYGGADGEHLRPEDKFSADGWLKTGDVGVISADGFLTLTDRAKDVIKSGGEWISSVELENALMAHPDVAEAAVVAVPDEKWGERPLATVVLKEGAEVGYEALKVFLADSGIAKWQLPERWTVIPAVPKTSVGKFDKKVIRKQYADGELDITQL

SEQ ID NO: 5 - protein, 560 aa, Erythrobacter citreus LAMA 915
MSRAECRDRLTAPGERFEIETIDIRGVPTRVWKHAPTNMRQVAMAARTHGDRLFAIYEDERVTYEAWFRAVARMAAELRERGVAKGDRVALAMRNLPEWPVAFFAATTIGAICVPLNAWWTGPELAFGLANSGAKLLVCDAERWERIAPHRGELPDLEHALVSRSDAPLEGAEQLEDLLGTPKDYAALPSAALPQVDIDPEDEATIFYTSGTTGQPKGALGTHRNLCTNIMSSAYNGAIAFLRRGEEPPAPVQKVGLTVIPLFHVTACSAGLMGYVVAGHTMVFMHKWDPVKAFQLIEREKVNLTGGVPTIAWQLLEHPERANYDLSSLEAVAYGGAPAAPELVRKIHEEFGALPANGWGMTETMATVTGHSSEDYLNRPDSCGPPVAVADLKIVGDDGVTELPVGEVGELWARGPMVVKGYWNRPEATAETFVDGWVRTGDLARLDEEGWCYIVDRAKDMIIRGGENIYSSEVENVLYDHPAVTDAALVAIAHPTLGEEPAAVVHLAPGMSATEDELREWVAARLAKFKVPVRIAFVQDTLPRNANGKILKKDLGAFFA

SEQ ID NO: 6 - protein, 700 aa, Saccharomyces cerevisiae
MVAQYTVPVGKAANEHETAPRRNYQCREKPLVRPPNTKCSTVYEFVLECFQKNKNSNAMGWRDVKEIHEESKSVMKKVDGKETSVEKKWMYYELSHYHYNSFDQLTDIMHEIGRGLVKIGLKPNDDDKLHLYAATSHKWMKMFLGAQSQGIPVVTAYDTLGEKGLIHSLVQTGSKAIFTDNSLLPSLIKPVQAAQDVKYIIHFDSISSEDRRQSGKIYQSAHDAINRIKEVRPDIKTFSFDDILKLGKESCNEIDVHPPGKDDLCCIMYTSGSTGEPKGVVLKHSNVVAGVGGASLNVLKFVGNTDRVICFLPLAHIFELVFELLSFYWGACIGYATVKTLTSSSVRNCQGDLQEFKPTIMVGVAAVWETVRKGILNQIDNLPFLTKKIFWTAYNTKLNMQRLHIPGGGALGNLVFKKIRTATGGQLRYLLNGGSPISRDAQEFITNLICPMLIGYGLTETCASTTILDPANFELGVAGDLTGCVTVKLVDVEELGYFAKNNQGEVWITGANVTPEYYKNEEETSQALTSDGWFKTGDIGEWEANGHLKIIDRKKNLVKTMNGEYIALEKLESVYRSNEYVANICVYADQSKTKPVGIIVPNHAPLTKLAKKLGIMEQKDSSINIENYLEDAKLIKAVYSDLLKTGKDQGLVGIELLAGIVFFDGEWTPQNGFVTSAQKLKRKDILNAVKDKVDAVYSSS

SEQ ID NO: 7 - protein, 536 aa, Citreicella sp. SE45
MSLSTEETARRRTLAEGAGYDALREGFRWPGAARVNMAEQVCDSWAAREPGRPAILDMRAGGAPEVVSYGALQALSRRVEAWFRGQGVARGDRVGVLLSQSPLCAAAHIAAWRMGAISVPLFKLFKHDALESRLGDSGARVVVSDDEGAAMLAPFGLSVVTEAGLPQDGATEPAADTGPEDPAIIIYTSGTTGKPKGALHGHRVLTGHLPGVEMSHDLLGQPGDVLWTPADWAWIGGLFDVLMPGLYLGVPVVAARMPRFEISECLRICQQASVRNVFFPPTAFRMLKSEGAELPGLRSVASGGEPLGAEMLAWGRKAFGVEINEFYGQTECNMVASSCGALFRARPGCIGKPAPGFHIAVIDEDGNETDGEGDVAIRRGAGSMLLEYWQKPQETADKFRGDWLVTGDRGTWEDGYLRFVGREDDVITSAGYRIGPTEIEDCLMTHPAVATVGVVGKPCPLRTELVKAYVVLRPGVEVRASELQAWVKERLATYSYPREIAFLDALPMTVTGKVIRKELKAIAAAERTAEAAGEVS

SEQ ID NO: 8 - protein, 513 aa, Bacillus subtilis (strain 168)
MNLVSKLEETASEKPDSIACRFKDHMMTYQELNEYIQRFADGLQEAGMEKGDHLALLLGNSPDFIIAFFGALKAGIVVVPINPLYTPTEIGYMLTNGDVKAIVGVSQLLPLYESMHESLPKVELVILCQTGEAEPEAADPEVRMKMTTFAKILRPTSAAKQNQEPVPDDTAVILYTSGTTGKPKGAMLTHQNLYSNANDVAGYLGMDERDNVVCALPMFHVFCLTVCMNAPLMSGATVLIEPQFSPASVFKLVKQQQATIFAGVPTMYNYLFQHENGKKDDFSSIRLCISGGASMPVALLTAFEEKFGVTILEGYGLSEASPVTCFNPFDRGRKPGSIGTSILHVENKVVDPLGRELPAHQVGELIVKGPNVMKGYYKMPMETEHALKDGWLYTGDLARRDEDGYFYIVDRKKDMIIVGGYNVYPREVEEVLYSHPDVKEAVVIGVPDPQSGEAVKGYVVPKRSGVTEEDIMQHCEKHLAKYKRPAAITFLDDIPKNATGKMLRRALRDILPQ

SEQ ID NO: 9 - protein, 694 aa, Saccharomyces cerevisiae
MTEQYSVAVGEAANEHETAPRRNIRVKDQPLIRPINSSASTLYEFALECFTKGGKRDGMAWRDIIDIHETKKTIVKRVDGKDKPIEKTWLYYELTPYITMTYEEMICVMHDIGRGLIKIGVKPNGENKFHIFASTSHKWMKTFLGCMSQGIPVVTAYDTLGESGLIHSMVETDSVAIFTDNQLLSKLAVPLKTAKNVKFVIHNEPIDPSDKRQNGKLYKAAKDAVDKIKEVRPDIKIYSFDEIIEIGKKAKDEVELHFPKPEDPACIMYTSGSTGTPKGVVLTHYNIVAGIGGVGHNVIGWIGPTDRIIAFLPLAHIFELTFEFEAFYWNGILGYANVKTLTPTSTRNCQGDLMEFKPTVMVGVAAVWETVRKGILAKINELPGWSQTLFWTVYALKERNIPCSGLLSGLIFKRIREATGGNLRFILNGGSAISIDAQKFLSNLLCPMLIGYGLTEGVANACVLEPEHFDYGIAGDLVGTITAKLVDVEDLGYFAKNNQGELLFKGAPICSEYYKNPEETAAAFTDDGWFRTGDIAEWTPKGQVKIIDRKKNLVKTLNGEYIALEKLESIYRSNPYVQNICVYADENKVKPVGIVVPNLGHLSKLAIELGIMVPGEDVESYIHEKKLQDAVCKDMLSTAKSQGLNGIELLCGIVFFEEEWTPENGLVTSAQKLKRRDILAAVKPDVERVYKENT

SEQ ID NO: 10 - protein, 504 aa, Bhargavaea cecembensis DSE10
MYTDHGWIMKRADITPDGTALIDVHTGQRWTYRELAGRTAAYMEQFRSAGLRKGERVAVLSHNRIDLFAVLFACAGRGLIYVPMNWRLSESELRYIVSDSGPSLLLHDHEHAGRAAGLGIPAALLDSVPATSVNLRTEQAAGRLDDPWMMIYTGGTTGRPKGVVLTFESVNWNAINTIISWNLSARDCTLNYMPLFHTGGLNALSLPILMAGGTVVIGRKFDPEEAIRALNDYRTTISLFVPTMHQAMLDTDLFWESDFPTVDVFLSGGAPCPQTVYDAYRKKGVRFREGYGMTEAGPNNFIIDPDTAMRKRGAVGKSMQFNEVRILDAKGRPCRAGEVGELHLRGRHLFSHYWNNEEATQEALKEGWFSTGDLASRDEDGDYFIVGRKKEMIISGGENIYPQEVEQCLIGHDGVREIAVIGIADRKWGERVVAFIVAQPGNIPKTEELLKHCAQTLGSYKVPKDFFFVQELPITDIGKIDKKQLAIMAEELKKEEMQHPGQSG

SEQ ID NO: 11 - protein, 602 aa, Deltaproteobacteria bacterium ADurb.Bin022
MHKFTLDKPDNLVDWWGESVTRFADRPLFGTKNKEGVYKWATYKEIGNRIDNLRAGLTQLGIGKDDVVGIIANNRPEWAVIGFATWGCLARYVPMYEAELVQVWKYIINDSGAKVLFVSNPAIYEKIKDFPKDIPTLKHIFIIESDGDNSMASLEKKGAAKPVAPKSPKAEDVAELIYTSGTTGNPKGVLLMHMNFTSNSHAGLKMYPELYENEVVSLTILPWAHVFGQTAELFAIIRLGGRMGLIESTKTIINDIVQIKPTFIIAVPTVFNRIYDGLWNKMNKDGGLARALFVMGVEAAKKKRILAEKGQSDLMTNFKVAVADKIVFKKIRERMGGRMLGSMTGSAAMNVEISKFFFDIGIPIYDCYGLTETSPGITMNGSQAYRIGSVGRPIDKVKVVIDSSVVEEGATDGEIIAYGPNVMKGYHNRPEDTKAALTPDGGFRTGDRGRLDKDGYLFITGRIKEQYKLENGKFCFPVSLEENICLASFVQQAVVYGLNRPYNVCIVVPDFDVLLDYAKEKGLPTDIKTLVEREDIIHMISEAVTGQLKGKFGGYEIPKKFIILPEAFSLDNGMLTQTMKLKRKVILDKLNDRIEALYKEDK

SEQ ID NO: 12 - protein, 534 aa, Alcaligenes xylosoxydans
MYSRIHEPHACTLTDALREWAASRPAAPWLEDSQGIAFTVGQAFTSSQRFASFLHHQLGVQPEERVGVFMSNSCAMVATTFGIGYLRATAVMLNTELRSSFLRHQLNDCQLATIVVDSALVEHVASLADELPHLRTLVVVGDAPAAVPERWRQVAWMDSSACAPWEGPAPRPEDIFCIMYTSGTTGPSKGVLMPHCHCALLGLGAIRSLEITEADKYYICLPLFHANGLFMQLGATVLAGIPAFLKQRFSASTWLADIRRSGATLTNHLGTTAMFVINQPPTEQDRDHRLRASLSAPNPAQHEAVFRERFGVKDVLSGFGMTEVGIPIWGRIGHAAPNAAGWAHEDRFEICIADPETDVPVLAGQVGEILVRPKVPFGFMAGYLNVPAKTVEAWRNLWFHTGDAGTRDEQGLITFVDRIKDCIRRRGENISATEVEVVVGQLPGVHEVAAYAVPAQGAGGEDEVMLALVPSEGAALDMADIVRQASAQLPRFAKPRYLRQMDSLPKTATGKIQRAVLRQQGSAGAYDAEAAPAR

SEQ ID NO: 13 - protein, 518 aa, Novosphingobium sp. MD-1
MQFTQGLERAVQHHPDVTATICRARSQTFAELYERVTGLAGCLASRSLAKGARIAVLALNSDHYLEVYLATAWAGGVIVPVNFRWSPAEIAYSLNDAGCVALMVDQHHAALVPTLREQCPGLQHIFLMGGTEESDDLPGLDALIAAAEPLQNAGAGGDDLLGIFYTGGTTGRPKGVMLSHANLCSSGLSMLAEGVFNEGAVGLHVAPMFHLADMLLTTCLVLRGCTHVMLPAFSPDAVLDHVARFGVTDTLVVPAMLQAIVDHPAIGNFDTSSLCNILYGASPASETLLRRTMAAFPDVRLTQGYGMTESAAFICALPWHQHVVDNDGPNRLRAAGRSTFDVHLQIVDPDDRELPRGEIGEIIVKGPNVMQGYYNMPEATAETLRGGWLHTGDMAWMDEEGYVFIVDRAKDMIISGGENIYSAEVENAVASHPAVAANAVIGIPHEQMGEAVHVALVLRPGSELSLEALQAHCRALIAGYKVPRSMEVRPSLPLSGAGKILKTELREPFWKGRDRAVG

SEQ ID NO: 14 - protein, 720 aa, Arabidopsis thaliana
MEDSGVNPMDSPSKGSDFGVYGIIGGGIVALLVPVLLSVVLNGTKKGKKRGVPIKVGGEEGYTMRHARAPELVDVPWEGAATMPALFEQSCKKYSKDRLLGTREFIDKEFITASDGRKFEKLHLGEYKWQSYGEVFERVCNFASGLVNVGHNVDDRVAIFSDTRAEWFIAFQGCFRQSITVVTIYASLGEEALIYSLNETRVSTLICDSKQLKKLSAIQSSLKTVKNIIYIEEDGVDVASSDVNSMGDITVSSISEVEKLGQKNAVQPILPSKNGVAVIMFTSGSTGLPKGVMITHGNLVATAAGVMKVVPKLDKNDTYIAYLPLAHVFELEAEIVVFTSGSAIGYGSAMTLTDTSNKVKKGTKGDVSALKPTIMTAVPAILDRVREGVLKKVEEKGGMAKTLFDFAYKRRLAAVDGSWFGAWGLEKMLWDALVFKKIRAVLGGHIRFMLVGGAPLSPDSQRFINICMGSPIGQGYGLTETCAGATFSEWDDPAVGRVGPPLPCGYVKLVSWEEGGYRISDKPMPRGEIVVGGNSVTAGYFNNQEKTDEVYKVDEKGTRWFYTGDIGRFHPDGCLEVIDRKKDIVKLQHGEYVSLGKVEAALGSSNYVDNIMVHADPINSYCVALVVPSRGALEKWAEEAGVKHSEFAELCEKGEAVKEVQQSLTKAGKAAKLEKFELPAKIKLLSEPWTPESGLVTAALKIKREQIKSKFKDELSKLYA

SEQ ID NO: 15 - protein, 534 aa, Bradyrhizobium sp. CI-41S
MDWSQHAIPPMRLEPRFGDRVVPAFVDRPASLWAMIADAVAQNGGGEALVCGDIRISWHEVARRAAKVAAGFAKLGLNSGDRVAILLGNRIEFVLTMFAAAHAGLVTVLLSTRQQKPEIAYVLNDCGARALVHEATLAERIPDAADIPGLAHRIAVSDDAASQFAVLLDHPPAPAPAAVSEEDTAMILYTSGTTGRPKGAMLAHCNIIHSSMVFASTLRLTQADRSIAAVPLAHVTGAVANITTMVRCAGTLIIMPEFKAAEYLKVAARERVSYTVMVPAMYNLCLLQPDFDSYDLSSWRIGGFGGAPMPVATIERLDAKIPGLKLANCYGATETTSPSTLMPGELTAAHIDSVGLPCPGAEIIVMGPDGRELPRGEIGELWIRSASVIKGYWNNPKATAESFTDGFWHSGDLGSVDAENFVRVFDRQKDMINRGGLKIYSAEVESVLAGHPAVIESAIIAKPCPVLGERVHAVIVTRTEVDAESLRAWCAERLSDYKVPETMTLTTTPLPRNANGKVVKRQLRETLAAGQAPA

SEQ ID NO: 16 - protein, 612 aa, Bradyrhizobium sp. CI-41S
MAGPAVLTVADTIARSFLLAVQTRGDRPAIREKKFGIWQPTSWREWLQISKDIAHGLHASGFRPGDVASIIANAVPEWVYADMGILCAGGVSSGIYPTDSTAQVEYLVNDSRTKIVFVEDEEQLDKVLACRARCPTLEKIVVFDMEGLSGFSDPMVLSFAEFAALGRNHAHGNAALWDEMTGSRTASDLAILVYTSGTTGPPKGAMHSNRSVTHQMRHANDLFPSTDSEERLVFLPLCHVAERVGGYYISIALGSVMNFAESPETVPDNLREVQPTAFLAVPRVWEKFYSGITIALKDATPFQNWMYGRALAIGNRMTECRLEGETPPLSLRLANRAAYWLVFRNIRRMLGLDRCRIALTGAAPISPDLIRWYLALGLDMREVYGQTENCGVATIMPTERIKLGSVGKAAPWGEVMICPKGEILIKGDFLFMGYLNQPERTAETIDAKGWLHTGDVGTIDNEGYVRITDRMKDIIITSGGKNVTPSEIENQLKFSPYVSDAVVIGDKRPYLTCLIMIDQENVEKFAQDHDIPFTNYASLCRAREIQDLIQREVEAVNTKFARVETIKKFYLIERQLTPEDEELTPTMKLKRSFVNKRYAAEIDAMYGARAVA

SEQ ID NO: 17 - protein, 541 aa, Thermus thermophilus
MEGERMNAFPSTMMDEELNLWDFLERAAALFGRKEVVSRLHTGEVHRTTYAEVYQRARRLMGGLRALGVGVGDRVATLGFNHFRHLEAYFAVPGMGAVLHTANPRLSPKEIAYILNHAEDKVLLFDPNLLPLVEAIRGELKTVQHFVVMDEKAPEGYLAYEEALGEEADPVRVPERAACGMAYTTGTTGLPKGVVYSHRALVLHSLAASLVDGTALSEKDVVLPVVPMFHVNAWCLPYAATLVGAKQVLPGPRLDPASLVELFDGEGVTFTAGVPTVWLALADYLESTGHRLKTLRRLVVGGSAAPRSLIARFERMGVEVRQGYGLTETSPVVVQNFVKSHLESLSEEEKLTLKAKTGLPIPLVRLRVADEEGRPVPKDGKALGEVQLKGPWITGGYYGNEEATRSALTPDGFFRTGDIAVWDEEGYVEIKDRLKDLIKSGGEWISSVDLENALMGHPKVKEAAVVAIPHPKWQERPLAVVVPRGEKPTPEELNEHLLKAGFAKWQLPDAYVFAEEIPRTSAGKFLKRALREQYKNYYGGA

SEQ ID NO: 18 - protein, 524 aa, Microbacterium oxydans
MVRSTYPDVEIPEVSIHDFLFGDLSEAELDTVALVDGMSGATTTYRQLVGQIDLFAGALAARGVGVGTTVGVLCPNVPAFATVFHGILRAGATATTINSLYTADEIANQLTDAGATWLVTVSPLLPGAQAAAEKLGFDADHVIVLDGAEGHPSLPALLGEGRQAPDVSFDPSTHLAVLPYSSGTTGRPKGVMLTHRNLVANVSQCQPVLGVDASDRVLAVLPFFHIYGMTVLLNFALRQRAGLATMPRFDLPEFLRIIAEHRTSWVFVAPPIAVALAKHPIVDQYDLSAVKVIFSGAAPLDGTLASAVANRLGCIVTQGYGMTETSPAVNLISEARTEIDRSTIGPLVPNTEARLVDPDSGEDVVVPAEGASEPGELWVRGPQVMVGYLNRPDATAEMLDADGWLHTGDVATVTHDGIYRIVDRLKELIKYKGYQVAPAVLEAVLLEHPAIADAAVIGAFDDDGQEVPKAFVVRQPDADLDADAVMAHVTSHVAPHEKVRQVEFIDVIPKSSSGKILRKDLRAR

SEQ ID NO: 19 - protein, 665 aa, Arabidopsis thaliana
MSLAADNVLLVEEGRPATAEHPSAGPVYRCKYAKDGLLDLPTDIDSPWQFFSEAVKKYPNEQMLGQRVTTDSKVGPYTWITYKEAHDAAIRIGSAIRSRGVDPGHCCGIYGANCPEWIIAMEACMSQGITYVPLYDSLGVNAVEFIINHAEVSLVFVQEKTVSSILSCQKGCSSNLKTIVSFGEVSSTQKEEAKNQCVSLFSWNEFSLMGNLDEANLPRKRKTDICTIMYTSGTTGEPKGVILNNAAISVQVLSIDKMLEVTDRSCDTSDVFFSYLPLAHCYDQVMEIYFLSRGSSVGYWRGDIRYLMDDVQALKPTVFCGVPRVYDKLYAGIMQKISASGLIRKKLFDFAYNYKLGNMRKGFSQEEASPRLDRLMFDKIKEALGGRAHMLLSGAAPLPRHVEEFLRIIPASNLSQGYGLTESCGGSFTTLAGVFSMVGTVGVPMPTVEARLVSVPEMGYDAFSADVPRGEICLRGNSMFSGYHKRQDLTDQVLIDGWFHTGDIGEWQEDGSMKIIDRKKNIFKLSQGEYVAVENLENTYSRCPLIAQIWVYGNSFESFLVGVVVPDRKAIEDWAKLNYQSPNDFESLCQNLKAQKYFLDELNSTAKQYQLKGFEMLKAIHLEPNPFDIERDLITPTFKLKRPQLLQHYKGIVDQLYSEAKRSMA

SEQ ID NO: 20 - protein, 559 aa, Brevibacterium yomogidense
MSWFDERPWLRTLGLTETEAVPLEPSTPLRDLADTVAAHPTTAAWTHYGQSATYAEFDRQTTAFAAYLAESGIRPGDAVAVYAQNSPHFPIATYGIWKAGAVVVPLNPMYRDELTHAFADADVKAIVVQKALYLMRVKEYAADLPLVVLAGDLDWAQDGPDAVFGAYADLPDVPLPDLRTVVDERLDTDFEPLTVRPEDPALIGYTSGTSGKAKGALHPHSSISSNSRMAARNAGLPQGAGVVSLAPLFHITGFICQMIASTANGSTLVLNHRFDPASFLDLLRQEKPAFMAGPATVYTAMMASPSFGADAFDSFHSIMSGGAPLPEGLVKRFEEKTGHYIGQGYGLTETAAQAVTVPHSLRAPVDPESGNLSTGLPQRDAMVRILDDDGNPVGPREVGEVAISGPMVATEYLGNPQATADSLPGGELRTGDVGFMDPDGWVFIVDRKKDMINASGFKVWPREVEDILYMHPAVREGAVVGVPDEYRGETVVAFVSLQPDSQATAEDIIAHCKEHLASYKAPVEVTIVDELPKTSSGKILRRTVRDEATQARQAQPDAH

SEQ ID NO: 21 - protein, 555 aa, Nocardioides simplex
MSFRYYRDLHPTFADRTEWALPTVLRHHAAERPDAVWLDCPEEGRTWTFAETLTAAERVGRSLLAAGAEPGDRVVLVAQNSSAFVRTWLGTAVAGLVEVPVNTAYEHDFLAHQVSTVEATLAVVDDVYAARFVAIAEAAKSIRKFWVIDTGSRDQALATLRDAGWEAAPFEELDEAATAPEVVDATLALPDVRPQDLASVLFTSGTTGPSKGVAMPHAQMYFFADECVSLVRLTPDDAWMSVTPLFHGNAQFMAAYPTLVAGARFVTRSRFSASRWVDQLRESRVTVTNFIGVMMDFIWKQDRRDDDADNPLRVVFAAPTAATLVGPMSERYGIEAFVEVFGLTETSAPIISPYGVDRPAGAAGLAADEWFDVRLVDPETDEEVGVGEIGELVVRPKVPFICSMGYFNMPDKTVEAWRNLWFHTGDALRRDEDGWFYFVDRFKDALRRRGENISSYEIETSILAHPAVVECAVIAVPASSEAGEDEVMAYVITGGDAPVPTPAELWAHCDGRIPSFAVPRYLRFVDEMPKTPSQRVQKAKLRALGVTPDTHDREA

SEQ ID NO: 22 - protein, 568 aa, Brevibacterium linens
MTVTEEFRAARDKLIELRSDYDAAREQFEWPRFDHFNFALDWFDKIAENNDKPALWIVEQDGSEGKWSFAELSARSNQVANHFRRAGIKRGDHVMVMLNNQVELWETMLAGIKLGAVLMPATTQLGPIDLTDRAERGHAEFVVAGAEDAAKFDDVDVEVVRIVVGGEPTRQQDYSYSDADDESTEFDPQGSSRADDLMLLYFTSGTTSKAKMVAHTHVSYPVGHLSTMYWMGLTPGDVHLNVASPGWAKHAWSNIFTPWIAEACVFLYNYSRFDANALMETMDRVGVTSFCAPPTVWRMLIQADLKHLKTPPTKALGAGEPLNPEIIDRVHSDWGVLIRDGFGQTESTLQIGNSPDQELKYGSMGKALPGFDVVLIDPATGEEGDEGEICLRLDPRPIGLTTGYWSNPEKTAEAFEGGVYHTGDVASRDEDGFITYVGRADDVFKASDYRLSPFELESVLIEHEAVAEAAVVPSPDPVRLAVPKAYVVVSSKFDADAETARSILAYCREHLAPYKRIRRLEFAELPKTISGKIRRVELRAREDQLHPFSGEPVVEGNEYADTDFDLKS

SEQ ID NO: 23 - protein, 515 aa, Pseudomonas putida
MNLGKIITRSARYWPDHTAVADSQTRLTYAQLERRSNRLASGLGALGVATGEHVAILAANRVELVEAEVALYKAAMVKVPINARLSLDEVVRVLEDSCSVALITDATFAQALAERRAALPMLRQVIALEGEGGDLGYAALLERGSEAPCSLDPADDALAVLHYTSGSSGVLKAAMLSFGNRKALVRKSIASPTRRSGPDDVMAHVGPITHASGMQIMPLLAVGACNLLLDRYDDRLLLEAIERERVTRLFLVPAMINRLVNYPDVERFDLSSLKLVMYGAAPMAPALVKKAIELFGPILVQGYGAGETCSLVTVLTEQDHLIEDGNYQRLASCGRCYFETDLRVVNEAFEDVAPGEIGEIVVKGPDIMQGYWRAPALTAEVMRDGYYLTGDLATVDAQGYVFIVDRKKEMIISGGFNVYPSEVEQVIYGFPEVFEAAVVGVPDEQWGEAVRAVVVLKPGAQLDAAELIERCGRALAGFKKPRGVDFVTELPKNPNGKVVRRLVREAYWQHSDRRI

SEQ ID NO: 24 - protein, 681 aa, Drosophila melanogaster
MNDLKPATSYRSTSLHDAVKLRLDEPSSFSQTVPPQTIPEFFKESCEKYSDLPALVWETPGSGNDGWTTLTFGEYQERVEQAALMLLSVGVEERSSVGILAFNCPEWFFAEFGALRAGAVVAGVYPSNSAEAVHHVLATGESSVCVVDDAQQMAKLRAIKERLPRLKAVIQLHGPFEAFVDHEPGYFSWQKLQEQTFSSELKEELLARESRIRANECAMLIFTSGTVGMPKAVMLSHDNLVFDTKSAAAHMQDIQVGKESFVSYLPLSHVAAQIFDVFLGLSHAGCVTFADKDALKGTLIKTFRKARPTKMFGVPRVFEKLQERLVAAEAKARPYSRLLLARARAAVAEHQTTLMAGKSPSIYGNAKYWLACRVVKPIREMIGVDNCRVFFTGGAPTSEELKQFFLGLDIALGECYGMSETSGAITLNVDISNLYSAGQACEGVTLKIHEPDCNGQGEILMRGRLVFMGYLGLPDKTEETVKEDGWLHSGDLGYIDPKGNLIISGRLKELIITAGGENIPPVHIEELIKKELPCVSNVLLIGDHRKYLTVLLSLKTKCDAKTGIPLDALREETIEWLRDLDIHETRLSELLNIPADLQLPNDTAALAATLEITAKPKLLEAIEEGIKRANKYAISNAQKVQKFALIAHEFSVATGELGPTLKIRRNIVHAKYAKVIERLYK

SEQ ID NO: 25 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 26 - protein, 390 aa, Dendrobium catenatum
MPSLESIRKAPRANGFASILAIGRANPENFIEQSTYPDFFFRITNSEHLVDLKKKFQRICDKTAIRKRHFVWNEEFITTNPCLHTFMDKSLDVRQEVAIREIPKLGAKAAAKAIQEWGQPKSRITHLIFCTTSGMDLPGADYQLTQILGLNPNVERVMLYQQGCFAGGTTLRLAKCLAESRKGARVLVVCAETTTVLFRGPSEEHQEDLVTQALFADGASALIVGADPDEAAHERASFVIVSTSQVLLPDSAGAIGGHVSEGGLLATLHRDVPKIVSKNVEKCLEEAFTPFGITDWNSIFWVPHPGGRAILDLVEERVGLKPEKLLVSRHVLAEYGNMSSVCVHFALDEMRKRSAIEGKATTGEGLEWGVVFGFGPGLTVETVVLRSVPL

SEQ ID NO: 27 - protein, 497 aa, Dictyostelium
MNNSNVKSSPSIVKEEIVTLDKDQQPLLLKEHQHIIISPDIRINKPKRESLIRTPILNKFNQITESIITPSTPSLSQSDVLKTPPIKSLNNTKNSSLINTPPIQSVQQHQKQQQKVQVIQQQQQPLSRLSYKSNNNSFVLGIGISVPGEPISQQSLKDSISNDFSDKAETNEKVKRIFEQSQIKTRHLVRDYTKPENSIKFRHLETITDVNNQFKKVVPDLAQQACLRALKDWGGDKGDITHIVSVTSTGIIIPDVNFKLIDLLGLNKDVERVSLNLMGCLAGLSSLRTAASLAKASPRNRILVVCTEVCSLHFSNTDGGDQMVASSIFADGSAAYIIGCNPRIEETPLYEVMCSINRSFPNTENAMVWDLEKEGWNLGLDASIPIVIGSGIEAFVDTLLDKAKLQTSTAISAKDCEFLIHTGGKSILMNIENSLGIDPKQTKNTWDVYHAYGNMSSASVIFVMDHARKSKSLPTYSISLAFGPGLAFEGCFLKNVV

SEQ ID NO: 28 - protein, 497 aa, Arachis hypogaea; identical to SEQ ID NO: 27

SEQ ID NO: 29 - protein, 394 aa, Spinacia oleracea
MASVDISEIHNVERAKGQANVLAIGTANPPNVMYQADYPDFYFRLTNSEHMTDLKAKFKRICEKTTIKKRYMHISEDILKEKPDLCDYNASSLDIRQVILAKEVPKVGKDAAMKAIEEWGQAMSKITHLIFCTTSGVDIPGADYQLTMLLGLNPSVKRYMLCQQGCHAGGTVLRLAKDLAENNYGSRVLVVCSENTTVCFRGPTETHPDSMVAQALFADGAGAVIVGAYPDESLNERPIFQIVSTAQTILPNSQGAIEGHLRQIGLAIQLLPNVPDLISNNIDKCLVEAFNPIGINDWNSIFWIAHPGGPAILGQVESKLGLQESKLTTTWHVLREFGNMSSACVFFIMDETRKRSLKEGKTTTGDGFDWGVLFGFGPGLTVETVVLRSFPLNQ

SEQ ID NO: 30 - protein, 389 aa, Elaeis guineensis
MSGLSRDMNPSLERSVGRAAVLGIGTANPPHVVEQSTFPDYYFKITNSEHMAHLKEKFTRICEKSKIAKRYTVLTDEFLVANPTLTSFNAPSLDTRQQLLDVEVPRLGAEAATRAIKDWGRPMSDLTHLIFCNSYGASIPGADYELVKLLGLPLSTRRVMLYQQCCYAGGTVIRLAKDLAENNRDARVLVVCCELNTVGIRGPCQSHLEDLVSQALFGDGAGALIIGADPRAGVERSIFEIVRTSQNIIAGSEGALVAKLREVGLVGRLKPEIPMHLSCSIEKLASEALNPVGIADWNEAFWVMHPGGRAILDELEKKLGLGEEKLAATREVLRDYGNMSSTSVLFVMEVMRRRSEERGLATAGEGLEWGVLLGFGPGLTMETVVLRCP

SEQ ID NO: 31 - protein, 392 aa, Vitis pseudoreticulata
MALVEEIRNAQRAKGPATVLAIGTATPDNCLYQSDFADYYFRVTKSEHMTELKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLYHQGCYAGGTVLRTAKDLAENNAGARVLVVCSEITVVTFRGPSENALDSLVGQALFGDGSAAVIVGSDPDISIERPLFQLVSAAQTFIPNSAGAIAGNLREVGLTFQLWPNVPTLISENIEKCLTKAFDPIGISDWNSLFWIAHPGGPAILDAVEAKLNLDKQKLKATRHILSEYGNMSSACVLFILDEMRKKSLKEGKTTTGEGLDWGVLFGFGPGLTIETVVLHSVQMDSN

SEQ ID NO: 32 - protein, 392 aa, Cannabis sativa
MASISVDQIRKAQRANGPATVLAIGTANPPTSFYQADYPDFYFRVTKNQHMTELKDKFKRICEKTTIKKRHLYLTEDRLNQHPNLLEYMAPSLNTRQDMLVVEIPKLGKEAAMKAIKEWGQPKSRITHLIFCSTNGVDMPGADYECAKLLGLSSSVKRVMLYQQGCHAGGSVLRIAKDLAENNKGARILTINSEITIGIFHSPDETYFDGMVGQALFGDGASATIVGADPDKEIGERPVFEMVSAAQEFIPNSDGAVDGHLTEAGLVYHIHKDVPGLISKNIEKSLVEALNPIGISDWNSLFWIVHPGGPAILNAVEAKLHLKKEKMADTRHVLSEYGNMSSVSIFFIMDKLRKRSLEEGKSTTGDGFEWGVLFGFGPGLTVETIVLHSLAN

SEQ ID NO: 33 - protein, 391 aa, Chenopodium quinoa
MASVQEIRNAQRADGPATILAIGTANPPNEMYQAEYPDFYFRVTESEHMTDLKKKFKRMCERSMIKKRYMHVTEELLKENPHMCDYNASSLNTRQDILATEVPKLGKEAAIKAIKEWGQPRSKITHVIFCTTSGVDMPGADYQLTKLLGLRPSVKRFMLYQQGCYAGGTVLRLAKDIAENNRGARVLVVCAEITVICFRGPTETHLDSMIGQALFGDGAGAVIVGADVDESIERPIFQLVWAAQTILPDSEGAIDGHLREVGLAFHLLKDVPGLISKNIEKALVEAFKPIGIDDWNSIFWVAHPGGPAILDQVESKLELKQDKLRDTRHVLSEFGNMSSACVLFILDEMRNRSLKEGKTTTGEGLDWGVLFGFGPGLTVETVMLHSVPITN

SEQ ID NO: 34 - protein, 389 aa, Ziziphus jujuba
MVTVDEIREAQRAKGPATIMAIGTATPPNAIDQSTFTDYYFRITNSDHKTDLKKKFKTICDKSMIKKRYLYLTEEHLKQNPNMSEYMAPSLDVRQEIVIAEVPKLGKEAANKAIKEWGQPKSKITHLVFSTISGVDAPGADYQLTKLLGLNPSVKRIMVYQQGCFAGGTSLRLAKDLAENNKGARVLVVCTEISAINFRGPSETYFDSNVGQILFGDGASAVVVGSDPLVGVEKPLFELVSASQTIIPDSEGNIEGHICEVGLTIRLSKKVPSLISNNIEKSLVEAFNPLGISDWNSIFWIAHPGGPAILDQIELKLGLKPEKLRASRHVLSEYGNMSSATVLFILDEMRKKSIEDGLKTPGEGLEWGVLFGFGPGLTVETVVLHSVTA

SEQ ID NO: 35 - protein, 319 aa, Marchantia polymorpha
MSRSRLIAQAVGPATVLAMGKAVPANVFEQATYPDFFFNITNSNDKPALKAKFQRICDKSGIKKRHFYLDQKILESNPAMCTYMETSLNCRQEIAVAQVPKLAKEASMNAIKEWGRPKSEITHIVMATTSGVNMPGAELATAKLLGLRPNVRRVMMYQQGCFAGATVLRVAKDLAENNAGARVLAICSEVTAVTFRAPSETHIDGLVGSALFGDGAAAVIVGSDPRPGIERPIYEMHWAGEMVLPESDGAIDGHLTEAGLVFHLLKDVPGLITKNIGGFLKDTKNLVGASSWNELFWAVHPGGPAILDQVEAKLELEKG

SEQ ID NO: 36 - protein, 393 aa, Caragana korshinskii
MAYLEEIREVQRARGPATILAIGTANPSNCIYQADFTDYYFRVTNSDHMTKLKAKLKRICENSMIKKRHVHLTEEILKENPNICTYKESSLDARQDMLIVEVPKLGEKAASKAIEEWGRPKSEITHLIFCSTSGVDMPGADYQLINLLGLKPSTKRFMLYHQGCFAGGTVLRLAKDLAENNAGARVLVVCSEITVVTFRGPSETHLDCLVGQALFGDGASSVIVGSDPDTSIERPLFHLVSASETILPNSEGAIEGHLREAGLMFQLKENVPQLIGENIEKSLEEMFHPLGISDWNSLFWISHPGGPAILKRIEETAGLNPEKLKATKHVLSEYGNMSSACVLFILDEMRKRSMEEGKSTTGEGLNWGVLFGFGPGLTMETIALHSANIDTGY

SEQ ID NO: 37 - protein, 389 aa, Glycine max
MVSVAEIRQAQRAEGPATILAIGTANPPNCVAQSTYPDYYFRITNSEHMTELKEKFQRMCDKSMIKRRYMYLNEEILKENPNMCAYMAPSLDARQDMVVVEVPKLGKEAAVKAIKEWGQPKSKITHLIFCTTSGVDMPGADYQLTKQLGLRPYVKRYMMYQQGCFAGGTVLRLAKDLAENNKGARVLVVCSEITAVTFRGPSDTHLDSLVGQALFGDGAAAVIVGSDPIPQVEKPLYELVWTAQTIAPDSEGAIDGHLREVGLTFHLLKDVPGIVSKNIDKALFEAFNPLNISDYNSIFWIAHPGGPAILDQVEQKLGLKPEKMKATRDVLSEYGNMSSACVLFILDEMRRKSAENGLKTTGEGLEWGVLFGFGPGLTIETVVLRSVAI

SEQ ID NO: 38 - protein, 394 aa, Humulus lupulus
MASVTVEQIRKAQRAEGPATILAIGTAVPANCFNQADFPDYYFRVTKSEHMTDLKKKFQRMCEKSTIKKRYLHLTEEHLKQNPHLCEYNAPSLNTRQDMLVVEVPKLGKEAAINAIKEWGQPKSKITHLIFCTGSSIDMPGADYQCAKLLGLRPSVKRVMLYQLGCYAGGKVLRIAKDIAENNKGARVLIVCSEITACIFRGPSEKHLDCLVGQSLFGDGASSVIVGADPDESVGERPIFELVSAAQTILPNSDGAIAGHVTEAGLTFHLLRDVPGLISQNIEKSLIEAFTPIGINDWNNIFWIAHPGGPAILDEIEAKLELKKEKMKASREMLSEYGNMSCASVFFIVDEMRKQSSKEGKSTTGDGLEWGALFGFGPGLTVETLVLHSVPTNV

SEQ ID NO: 39 - protein, 389 aa, Humulus lupulus
MVTVEEVRKAQRAEGPATILAIGTATPANCILQSEYPDYYFRITNSEHKTELKEKFKRMCDKSMIRKRYMHLTEEILKENPNLCAYEAPSLDARQDMVVVEVPKLGKEAATKAIKEWGQPKSKITHVVFCTTSGVDMPGADYQLTKLLGLRPSVKRLMMYQQGCFAGGTVLRVAKDLAENNKGARVLVVCSEITAVTFRGPNDTHLDSLVGQALFGDGSAALIIGADPTPEIEKPIFELVSAAQTILPDSDGAIDGHLREVGLTFHLLKDVPGLISKNIEKSLVEAFKPLGISDWNSLFWIAHPGGPAILDQVESKLALKPEKLRATRHVLGEYGNMSSACVLFILDEMRRKCAEDGLKTTGEGLEWGVLFGFGPGLTVETVVLHSVGI

SEQ ID NO: 40 - protein, 396 aa, Trema orientale
MASVTVDEIRKAQRAEGPATVLAIGTATPHNCVSQADYPDYYFRITNSEHMTELKEKFKRMCEKSMIKKRYMHLTEEILKENPKMCEYMAPSLDARQDMVVVEVPKLGKEAATKAIKEWGLPKSKITHLVFCTTSGVDMPGADYQLTKLLGLRPSVKRLMMYQQGCFAGGTVLRLAKDLAENNRGARVLVVCSEITAVTFRGPSDTHLDSMVGQALFGDGAAAVIVGADPDPSAGERPLFEMVSAAQTILPDSEGAIDGHLREAGLTFHLLKDVPGLISKNIEKSLTEAFSPLGISDWNSLFWIAHPGGPAILDQVEAKLKLKEEKLRATRHVLSEYGNMSSACVLFILDEMRKKSAEDGKPTTGEGLDWGVLFGFGPGLTVETVVLHSVAATATN

SEQ ID NO: 41 - protein, 396 aa, Plumbago indica
MAPAVQSQSHGGAYRSNGERSKGPATVLAIATAVPPNVYYQDEYADFFFRVTNSEHKTAIKEKFNRVCGTSMIKKRHMYFTEKMLNQNKNMCTWDDKSLNARQDMVIPAVPELGKEAALKAIEEWGKPLSNITHLIFCTTAGNDAPGADFRLTQLLGLNPSVNRYMIYQQGCFAGATALRIAKDLAENNKGARVLIVCCEIFAFAFRGPHEDHMDSLICQLLFGDGAAAVIVGGDPDETENALFELEWANSTIIPQSEEAITLRMREEGLMIGLSKEIPRLLGEQIEDILVEAFTPLGITDWSSLFWIAHPGGKAILEALEKKIGVEGKLWASWHVLKEYGNLTSACVLFAMDEMRKRSIKEGKATTGDGHEYGVLFGVGPGLTVETVVLKSVPLN

SEQ ID NO: 42 - protein, 398 aa, Artemisia annua
MASLTDIAAIREAQRAQGPATILAIGTANPANCVYQADYPDYYFRITKSEHMVDIKEKFKRMCDKSMIRKRYMHLTEEYLKENPSLCEYMAPSLDARQDVVVVEVPKLGKEAATKAIKEWGQPKSKITHLIFCTTSGVDMPGADYQLTKLLGLRPSVKRFMMYQQGCFAGGTVLRLAKDLAENNKDARVLVVCSEITAVTFRGPNDTHLDSLVGQALFGDGAAAVIVGSDPDLTKERPLFEMISAAQTILPDSEGAIDGHLREVGLTFHLLKDVPGLISKNIEKALTQAFSPLGISDWNSIFWIAHPGGPAILDQVELKLGLKEEKMRATRHVLSEYGNMSSACVLFIIDEMRKKSAEEGAATTGEGLDWGVLFGFGPGLTVETVVLHSLPTTISVVN

SEQ ID NO: 43 - protein, 397 aa, Actinidia chinensis var. chinensis
MAPSLEEILRAQRSQGPAEILGIGTATPPNCYDQADFPDFYFRVTNSEHMTHLKDKFKQICEKSTVKKRYMYLTEEILKDNPSLCSYMGRSLDVRQNMVMTEVPKLGKEAAAKAIKEWGQPKSKITHLVFCTTSGVDMPGADYHLTKLLGLQPSVKRIMMYQSSCYGGGTGLRLAKDLAENNAGARVLLVCSEISAINFRGPPDTPARLDKLVAQALFGDGAAAVIVGADPDTSIERSLFQLISASQTIVPGSNGGIMGTFGEAGLMCHLIKDVPRLISSNIEKCLMDAFTPIGINDWNSIFWIAHPGGPAILDMVEEKIGLEEEKLRATRHILSEYGNMSSVCVLFILDEMRKKSAEEGKLTTGEGLEWGVLFGFGAGITVETVVLRSMSISNTTH

SEQ ID NO: 44 - protein, 389 aa, Rhododendron dauricum
MVTVEDVRKAQRAEGPATVMAIGTATPPNCVDQSTYPDFYFRITNSEHKAELKEKFQRMCDKSMIKKRYMYLTEEILKENPSVCEYMAPSLDARQDMVVVEVPKLGKEAATKAIKEWGQPKSKITHLVFCTTSGVDMPGADYQLTKLLGLRPSVKRLMMYQQGCFAGGTVLRLAKDLAENNKGARVLVVCSEITAVTFRGPSDTHLDSLVGQALFGDGAAAIIVGADPVPEVEKPLFELVSAAQTILPDSDGAIDGHLREVGLTFHLLKDVPGLISKNIEKALTEAFQPLGISDWNSIFWIAHPGGPAILDQVELKLSLKPEKLRATRHVLSEYGNMSSACVLFILDEMRRKSAEEGLKTTGEGLEWGVLFGFGPGLTVETVVLHSLCT

SEQ ID NO: 45 - protein, 380 aa, Chenopodium quinoa
MASASMNPATILAIGTANPPNVMCQSDYPDYHFRTTNSDHLTDLKAKFKRICDKSMIRKRHFYMNEEILKENPHLGDNNASSIGTRQALCANEIPKLGKEAAEKAIKEWGKPKSMITHLIFGTNSDFDLPGADFRLAKLLGLQPTVKRFILPLGACHAGGTALRIAKDIAENNRGARVLVICSESTAISFHAPSETHLVSLAIFGDGAGAMIVGTDPDEPSERPLFQLVSAGQITLPDSEDGIQARLSEIGMTIHLSPDVPKIIAKNIQTLLSESFDHIGISNWNSIFWVAHPGGPAILDKVEAKLELETSKLSTSRHILSEYGNMWGASVIFVMDEMSKRSLKEGKSTTGEGCEWGVLVAFGPGITVETIVLRSMPINY

SEQ ID NO: 46 - protein, 388 aa, Cajanus cajan
MVSVEDIRKAQRAEGPATVMAIGTATPPNCVDQSTYPDYYFRITNSEHKTELKEKFKRMCDKSMIKKRYMYLNEEILKENPSVCEYMAPSLDARQDMVVVEVPKLGKEAATKAIKEWGQPKSKITHLIFCTTSGVDMPGADYQLTKLLGLRPSVKRYMMYQQGCFAGGTVLRLAKDLAENNKGARVLVVCSEITAVTFRGPSDTHLDSLVGQALFGDGAAAVIVGSDPLPVEKPFFELVWTAQTILPDSEGAIDGHLREVGLTFHLLKDVPGLISKNIEKALVEAFQPLGISDYNSIFWIAHPGGPAILDQVEAKLGLKPEKMEATRHVLSEYGNMSSACVLFILDQMRKKSIENGLGTTGEGLEWGVLFGFGPGLTVETVVLRSVTV

SEQ ID NO: 47 - protein, 393 aa, Lonicera japonica
MGSVTVEEIRKAQRAQGPATVLAIGTATPANCVYQADYPDFYFRITKSEHKAELKEKFKRMCEKSMIRKRYMHLNEEILKENPGICEYMAPSLDARQDMVVVEVPKLGKEAATKAIKEWGQPKSKITHLVFCTTSGVDMPGADYQLTKLLGLRPSVKRLMMYQQGCFAGGTVLRLAKDLAENNAGARVLVVCSEITAVTFRGPSDTHLDSLVGQALFGDGAAAVIIGADPDKSVERPLFELVSAAQTILPDSDGAIDGHLREVGLTFHLLKDVPGLISKNIEKSLKEAFAPIGITDWNSLFWIAHPGGPAILDQVEIKLGLKEEKLRPTRHVLSEYGNMSSACVLFILDELRKKSIEEGKATTGDGLEWGVLFGFGPGLTVETVVLHSVPASI

SEQ ID NO: 48 - protein, 391 aa, Ruta graveolens
MESLKEMRKAQMSEGPAAILAIGTATPNNVYMQADYPDYYFRMTKSEHMTELKDKFRTLCEKSMIRKRHMCFSEEFLKANPEVSKHMGKSLNARQDIAVVETPRLGNEAAVKAIKEWGQPKSSITHLIFCSSAGVDMPGADYQLTRILGLNPSVKRMMVYQQGCYAGGTVLRLAKDLAENNKGSRVLVVCSELTAPTFRGPSPDAVDSLVGQALFADGAAALVVGADPDSSIERALYYLVSASQMLLPDSDGAIEGHIREEGLTVHLKKDVPALFSANIDTPLVEAFKPLGISDWNSIFWIAHPGGPAILDQIEEKLGLKEDKLRASKHVMSEYGNMSSSCVLFVLDEMRSRSLQDGKSTTGEGLDWGVLFGFGPGLTVETVVLRSVPIEA

SEQ ID NO: 49 - protein, 397 aa, Physcomitrella patens subsp. Patens
MASAGDVTRAALPRAQPRAEGPACVLGIGTAVPPAEFLQSEYPDFFFNITNCGEKEALKAKFKRICDKSGIRKRHMFLTEEVLKANPGICTYMEPSLNVRHDIVVVQVPKLAAEAAQKAIKEWGGRKSDITHIVFATTSGVNMPGADHALAKLLGLKPTVKRVMMYQTGCFGGASVLRVAKDLAENNKGARVLAVASEVTAVTYRAPSENHLDGLVGSALFGDGAGVYVVGSDPKPEVEKPLFEVHWAGETILPESDGAIDGHLTEAGLIFHLMKDVPGLISKNIEKFLNEARKPVGSPAWNEMFWAVHPGGPAILDQVEAKLKLTKDKMQGSRDILSEFGNMSSASVLFVLDQIRHRSVKMGASTLGEGSEFGFFIGFGPGLTLEVLVLRAAPNSA

SEQ ID NO: 50 - protein, 387 aa, Rubus idaeus
MGSVAKEAKYPATILAIATANPANCYHQKDYPDFLFRVTKSEDKTELKDKFKRICEKSMVKKRYLGITEESLNANPNICTYKAPSLDSRQDLLVHEVPKLGKEAALKAIEEWGQPISSITHLIFCTASCVDMPGADFQLVKLLGLDPTIKRFMIYQQGCFAGGTVLRIAKDVAENNAGARLLIVCCEITTMFFQQPSENHLDVLVGQALFSDGAAALIVGTNPDPKSERQLFDIMSVRETIIPNSEHGVVAHLREMGFEYYLSSEVPKLVGGKIEEYLNKGFEGIGVDGDWNSLFYSIHPGGPAILNKVEEELGLKEGKLRATRHVLSEFGNMGAPSVLFILDEIRKRSMEEGKATTGEGFEWGVLIGIGPGLTVETVVLRSVSTAN

SEQ ID NO: 51 - protein, 419 aa, Marchantia polymorpha subsp. Ruderalis
MATRVLSSQENFEKLMADLARPNGHVYSQSQSQSGSGQNGAGTSIVAKNTASILAIGKALPPNRICQSTYTDFYFRVTHCSHKTELKNRMQRICDKSGINTRYLLLDEEALKEHSEFYTPGQASIEQRHDLLEEAVPKLAAQAAASALEEWGRPACDVTHLIVVTLSGVAIPGADVRLVKLLGLREDVSRVMLYMLGCYAGVTALRLAKDLAENNPGSRVLIACSEMTATTFRAPSEKSMYDIVGASLFGDGAVGVIVGAKPRPGIERSIFEIHWAGVSLAPDTEHVVQGKLKPDGLYFFLDKSLPGLVGKHIAPFCRSLLDHAPENLNLGFNEVFWAVHPGGPAILNTVEEQLLLNSEKLRASRDVLANYGNVSASSVLYVLDELRHRPGQEEWGAALAFGPGITFEGVLLRRNVNHR

SEQ ID NO: 52 - protein, 388 aa, Oryza sativa
MGKQGGQQLVAAILGIGTAVPPYVLPQSSFPDYYFDISNSNHLLDLKAKFADICEKTMIDKRHVHMSDEFLRSNPSVAAYNSPSINVRQNLTDVTVPQLGAAAARLAIADWGRPACEITHLVMCTTVSGCMPGADFEVVKLLGLPLTTKRCMMYHIGCHGGGTALRLAKDLAENNPGGRVLVVCSEVVSMVFRGPCESHMGNLVGQALFGDAAGAVVVGADPVEANGERTLFEMVSAWQDIIPETEEMVVAKLREEGLVYNLHRDVAARVAASMESLVKKAMVEKDWNEEVFWLVHPGGRDILDRVVLTLGLRDDKVAVCREVMRQHGNTLSSCVIVAMEEMRRRSADRGLSTAGEGLEWGLLFGFGPGLTVETILLRAPPCNQAQAV

SEQ ID NO: 53 - protein, 384 aa, Punica granatum
MGYSQQAKGPATIMAIGTAIPSYVVYQADFPDYYFRLSGCDHMTELKEKFIRICEKSTIRKRHMHLTEEILKQNPAILTYDGPSLNVRQQLVASEVPKLAMEAASKAIEEWGQPVWKITHLVFSSVVGAATPGADYKLIKLLGLEPSVKRVPLYQQGCYVGGTALRIAKDLAENNASARVLVVCVDNTISSFRGPSKHITNLVGQALFSDGASAAIVGADPIPSVERPIFQIAHTSMHLVPDSDSEVTLDFLDAGLIVHVSEKVPSLIADNLEKSLVEALGPTGINDWNSLFWAAHPGGPKILDMIEAKLGLRKEKLRATRTVLREYGNMIGACLLFILDEIRQNSLEAGMATTGEGFDWGILLGFGPGLTVEAVVLRSFPIAK

SEQ ID NO: 54 - protein, 393 aa, Citrus x microcarpa
MAKVKNFLNAKRSKGPASILAIGTANPPTCFNQSDYPDFYFRVTDCEHKTELKDKFKRICDRSAVKKRYLHVTEEVLKENPSMRSYNAPSLDARQALLIEQVPKLGKEAAAKAIKEWGQPLSKITHLVFSAMAGVDIPGADLRLMNLLGLEPSVKRLMIYSQGCFIGGAAIRCAKDFAENNAGARVLVVFSDIMNMYFHEPQEAHLDILVGQAVFGDGAAAVIVGADPEVSIERPLFHVVSSTQMSVPDTNKFIRAHVKEMGMELYLSKDVPATVGKNIEKLLVDAVSPFGISDWNSLFYSVHPGGRAILDQVELNLGLGKEKLRASRHVLSEYGNMGGSSVYFILDEIRKKSMQEAKPTTGDGLEWGVLFAIGPGLTVETVILLSVPIDSAC

SEQ ID NO: 55 - protein, 412 aa, Rhododendron dauricum
MALVNHRENVKGRAQILAIGTANPKNCFRQVDYPDYYFRVTKSDHLIDLKAKFKRMCEKSMIEKRYMHVNEEILEQNPSMNHGGEKMVSSLDVRLDMEIMEIPKLAAEAATKAMDEWGQPKSRITHLVFHSTLGTVMPGVDYELIKLLGLNPSVKRFMLYHLGCYGGGTVLRLAKDLAENNPGSRVLVLCCEMMPSGFHGPPSLQHAHLDILTGHAIFGDGAGAVIVGCVDPSGGTNGVVERGVRRYEQPLFEIHSAYQTVLPDSKDAVGGRLREAGLIYYLSKRLSNDVSGKIDECCLAEAFSAAIKDNFEDWNSLFWIVHPAGRPILDKLDAKLGLNKEKLRASRNVLRDYGNMWSSSVLFVLDEMRKGSIAQRKTTTGEGFEWGVLLGFGPGVTVETVVLRSVPTAKLK

SEQ ID NO: 56 - protein, 389 aa, Curcuma zedoaria
MEANGYRITHSADGPATILAIGTANPTNVVDQNAYPDFYFRVTNSEHLQELKAKFRRICEKAAIRKRHLYLTEEILRENPSLLAPMAPSFDARQAIVVEAVPKLAKEAAEKAIKEWGRPKSDITHLVFCSASGIDMPGSDLQLLKLLGLPPSVNRVMLYNVGCHAGGTALRVAKDLAENNRGARVLAVCSEVTVLSYRGPHPAHIESLFVQALFGDGAAALVVGSDPVDGVERPIFEIASASQVMLPESEEAVGGHLREIGLTFHLKSQLPSIIASNIEQSLTTACSPLGLSDWNQLFWAVHPGGRAILDQVEARLGLEKDRLAATRHVLSEYGNMQSATVLFILDEMRNRSAAEGHATTGEGLDWGVLLGFGPGLSIETVVLHSCRLN

SEQ ID NO: 57 - protein, 391 aa, Garcinia mangostana
MAPAMDSAQNGHQSRGSANVLAIGTANPPNVILQEDYPDFYFKVTNSEHLTDLKEKFKRICVKSKTRKRHFYLTEQILKENPGIATYGAGSLDSRQKILETEIPKLGKEAAMVAIQEWGQPVSKITHVVFATTSGFMMPGADYSITRLLGLNPNVRRVMIYNQGCFAGGTALRVAKDLAENNKGARVLVVCAENTAMTFHGPNENHLDVLVGQAMFSDGAAALIIGANPNLPEERPVYEMVAAHQTIVPESDGAIVAHFYEMGMSYFLKENVIPLFGNNIEACMEAAFKEYGISDWNSLFYSVHPGGRAIVDGIAEKLGLDEENLKATRHVLSEYGNMGSACVIFILDELRKKSKEEKKLTTGDGKEWGCLIGLGPGLTVETVVLRSVPIA

SEQ ID NO: 58 - protein, 391 aa, Arachis hypogaea
MGSLGATQEGNGAKGVATILAIGTANPPNIIRQDDYPDFYFRATKSNHMLHLKEKFQRLCKNSMIEKRHFLYNEDLLMENPNIVTYGASSLNTRQNILIKEVPKLGKEAALKAINEWGQPLSEITHLIFYTTSCFGNMPGPDYHLAKLLGLKPTVNRHMIFNNGCHGGGAVLRVAKDIVENNAGSRVLVVWVETMVASFHGPNPNHMDVLVGQALFGDGAGALIIGTNPKPCIECPLFELVLASQTTIPNTESSINGNIQEMGLVYYLGKEIPIAISENIDKCLINAFRESSVDWNSLFYAIHPGGPSILNRIEEKLGLKKEKLRASRKVLSQYGNMWSPGVIFVLDELRNWSKIEGKSTCGEGKEWGVLVGFGPGLSLELLVLRSFCFDG

SEQ ID NO: 59 - protein, 397 aa, Aquilaria sinensis
MAAQPVEWVRKADRAAGPAAVLAMATANPSNFYLQSDFPDFYFRVTRSDHMSDLKEKFKRICKKTTVRKRHMILTEEILNKNPAIADYWSPSLAARHDLALANIPQLGKEAADKAIKEWGQPKSKITHLVFCTSAGVLMPGADYQLTMLLGLNPSISRLMLHNLGCYAGGTALRVAKDLAENNGGARVLVVCSEANLLNFRGPSETHIDALITQSLFADGAAALIVGSDPDLQTESPLYELISASQRILPESEDAIVGRLTEAGLVPYLPKDIPKLVSTNIRSILEDALAPTGVQDWNSIFWIIHPGMPAILDQTEKLLQLDKEKLKATRHVLSEFGNMFSATVLFILDQLRKGAVAEGKSTTGEGCEWGVLFSFGPGFTVETVLLRSVATATLTDA

SEQ ID NO: 60 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 61 - protein, 333 aa, Stachybotrys bisbyi
MPATRTPIHPEAAAYKNPRYQSGPLSVIPKSFVPYCELMRLELPHGNFLGYFPHLVGLLYGSSASPARLPANEVAFQAVLYIGWTFFMRGAGCAWNDVVDQDFDRKTTRCRVRPVARGAVSTTSANIFGFAMVALAFACISPLPAECQRLGLMTTVLSIIYPFCKRVTNFAQVILGMTLAINFILAAYGAGLPAIEAPYTVPTICVTTAITLLVVFYDVVYARQDTADDLKSGVKGMAVLFRNYVEILLTSITLVIAGLIATTGVLVDNGPYFFVFSVAGLLAALLAMIGGIRYRIFHTWNSYSGWFYALAIFNLLGGYLIEYLDQVPMLNKA

SEQ ID NO: 62 - protein, 329 aa, Stachybotrys chartarum
MSAKVSPMAYTNPRYETGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 63 - protein, 329 aa, Stachybotrys chartarum
MSAKVSPMAYTNPRYERGPLSLIPKPIVPYFELMRFELPHGYYLGYFPHLVGIMYGASAGPERLPARDLVFQALLYVGWTFAMRGAGCAWNDNIDQDFDRKTERCRTRPIARGAVSTTAGHVFAVAGVALAFLCLSPLPTECHQLGVLVTVLSVIYPFCKRFTNFAQVILGMTLAANFILAAYGAGLPALEQPYTRPTMSATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 64 - protein, 329 aa, Stachybotrys chlorohalonata
MSPKVSSMPYTNPRYESGPLSLIPKSIVPYFELMRFELPHGYYLGYFPHLVGIMYGASAGPERLPARDLVFQALLYVGWTFAMRGAGCAWNDNIDQDFDRKTERCRTRPIARGAVSTTAGHIFAVAGVALAFLCLSPLPTECHQLGVLVTVLSVIYPFCKRFTNFAQVILGMTLAANFILAAYGAGLPALEQPYTRPTMFATLAITLLVVFYDVVYARQDTADDLKSGVKGMAVLFRNHIEVLLAVLTCTIGGLLAATGVSVGNGPYYFLFSVAGLTVALLAMIGGIRYRIFHTWNGYSGWFYVLAIINLMSGYFIEYLDNAPILARGS

SEQ ID NO: 65 - protein, 322 aa, Cannabis sativa
MAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 66 - protein, 357 aa, Streptomyces actuosus
MTTEVTSFTGAGPHPAASVRRITDDLLQRVEDKLASFLTAERDRYAAMDERALAAVDALTDLVTSGGKRVRPTFCITGYLAAGGDAGDPGIVAAAAGLEMLHVSALIHDDILDNSAQRRGKPTIHTLYGDLHDSHGWRGESRRFGEGIGILIGNLALVYSQELVCQAPPAVLAEWHRLCSEVNIGQCLDVCAAAEFSADPELSRLVALIKSGRYTIHRPLVMGANAASRPDLAAAYVEYGEAVGEAFQLRDDLLDAFGDSTETGKPTGLDFTQHKMTLLLGWAMQRDTHIRTLMTEPGHTPEEVRRRLEDTEVPKDVERHIADLVEQGRAAIADAPIDPQWRQELADMAVRAAYRTN

SEQ ID NO: 67 - protein, 417 aa, Artificial Sequence
  Synthetic Construct
MAFKLAQRLPKSVSSLGSQLSKNAPNQLAAATTSQLINTPGIRHKSRSSAVPSSLSKSMYDHNEEMKAAMKYMDEIYPEVMGQIEKVPQYEEIKPILVRLREAIDYTVPYGKRFKGVHIVSHFKLLADPKFITPENVKLSGVLGWCAEIIQAYFCMLDDIMDDSDTRRGKPTWYKLPGIGLNAVTDVCLMEMFTFELLKRYFPKHPSYADIHEILRNLLFLTHMGQGYDFTFIDPVTRKINFNDFTEENYTKLCRYKIIFSTFHNTLELTSAMANVYDPKKIKQLDPVLMRIGMMHQSQNDFKDLYRDQGEVLKQAEKSVLGTDIKTGQLTWFAQKALSICNDRQRKIIMDNYGKEDNKNSEAVREVYEELDLKGKFMEFEEESFEWLKKEIPKINNGIPHKVFQDYTYGVFKRRPE

SEQ ID NO: 68 - protein, 296 aa, Artificial Sequence
  Synthetic Construct
MAFDFKRYMVEKADSVNKALEAVVQMKEPLKIHESMRYSLLAGGKRVRPMLCIAACELVGGEESTAMPAACAVEMIHTMSLMHDDLPCMDNDDLRRGKPTNHKVFGEDVAVLAGDALLSLAFEHVAVATRGSAPERILRALGQLAKSIGAEGLVAGQVVDICSEGMAEVGLDHLEFIHLHKTAALLQGSVVMGAILGGAKEEEVERLRKFAKCIGLMFQVVDDILDVTKSSHELGKTAGKDLVADKTTYPKLLGVQKSKEFADDLNREAQEQLLHFDSHKAAPLIAIANYIAYRNN

SEQ ID NO: 69 - protein, 271 aa, Artificial Sequence
  Synthetic Construct
MAQNHSYWAAIEADIDTYLKKSIAIRSPETVFEPMHHLTFAAPRTAASAICVAACELVGGERSQAIATASAIHIMHAAAYAHEHLPLTDRPRPNSKPAIQHKYGPNIELLTGDGMASFGFELLAGSIRSDHPNPERILRVIIEISRASGSEGIIDGFYREKEIVDQHSRFDFIEYLCRKKYGEMHACAAASGAILAGGAEEEIQKLRNFGHYAGTLIGLLHKKIDTPQIQNVIGKLKDLALKELEGFHGKNVELLCSLVADASLCEAELEV

SEQ ID NO: 70 - protein, 296 aa, Artificial Sequence
  Synthetic Construct
MAFDFKAYMIGKANSVNKALEDAVLVREPLKIHESMRYSLLAGGKRVRPMLCIAACELFGGTESVAMPSACAVEMIHTMSLMHDDLPCMDNDDLRRGKPTNHKVFGEDVAVLAGDALLAFAFEHIATATKGVSSERIVRVVGELAKCIGSEGLVAGQVVDVCSEGIADVGLEHLEFIHIHKTAALLEGSVVLGAIVGGANDEQISKLRKFARCIGLLFQVVDDILDVTKSSQELGKTAGKDLVADKVTYPKLLGIDKSREFAEKLNREAQEQLAEFDPEKAAPLIALANYIAYRDN

SEQ ID NO: 71 - protein, 263 aa, Artificial Sequence
  Synthetic Construct
MAMKSNSWANIESDIQTHLKKSIPIRAPEDVFEPMHYLTFAAPRTTAPALCIAACEVVGGDGDQAMAAAAAIHLVHAAAYAHENLPLTDRRRPKPPIQHKFNSNIELLTGDGIVPYGFELLAKSMDSNNSDRILRVIIEITQAAGSKGIIDGQFRELDVIDSEINMGLIEYVCKKKEGELNACGAACGAILGGGSEEEIGKLRKFGLYAGMIQGLVHGVGKNREEIQELVRKLRYLAMEELKSLKNRKIDTISSLLETDLCSV

SEQ ID NO: 72 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 73 - protein, 713 aa, Saccharomyces cerevisiae
MSPSAVQSSKLEEQSSEIDKLKAKMSQSASTAQQKKEHEYEHLTSVKIVPQRPISDRLQPAIATHYSPHLDGLQDYQRLHKESIEDPAKFFGSKATQFLNWSKPFDKVFIPDSKTGRPSFQNNAWFLNGQLNACYNCVDRHALKTPNKKAIIFEGDEPGQGYSITYKELLEEVCQVAQVLTYSMGVRKGDTVAVYMPMVPEAIITLLAISRIGAIHSVVFAGFSSNSLRDRINDGDSKVVITTDESNRGGKVIETKRIVDDALRETPGVRHVLVYRKTNNPSVAFHAPRDLDWATEKKKYKTYYPCTPVDSEDPLFLLYTSGSTGAPKGVQHSTAGYLLGALLTMRYTFDTHQEDVFFTAGDIGWITGHTYVVYGPLLYGCATLVFEGTPAYPNYSRYWDIIDEHKVTQFYVAPTALRLLKRAGDSYIENHSLKSLRCLGSVGEPIAAEVWEWYSEKIGKNEIPIVDTYWQTESGSHLVTPLAGGVTPMKPGSASFPFFGIDAVVLDPNTGEELNTSHAEGVLAVKAAWPSFARTIWKNHDRYLDTYLNPYPGYYFTGDGAAKDKDGYIWILGRVDDVVNVSGHRLSTAEIEAAIIEDPIVAECAVVGFNDDLTGQAVAAFVVLKNKSNWSTATDDELQDIKKHLVFTVRKDIGPFAAPKLIILVDDLPKTRSGKIMRRILRKILAGESDQLGDVSTLSNPGIVRHLIDSVKL

SEQ ID NO: 74 - protein, 683 aa, Saccharomyces cerevisiae
MTIKEHKVVYEAHNVKALKAPQHFYNSQPGKGYVTDMQHYQEMYQQSINEPEKFFDKMAKEYLHWDAPYTKVQSGSLNNGDVAWFLNGKLNASYNCVDRHAFANPDKPALIYEADDESDNKIITFGELLRKVSQIAGVLKSWGVKKGDTVAIYLPMIPEAVIAMLAVARIGAIHSVVFAGFSAGSLKDRVVDANSKVVITCDEGKRGGKTINTKKIVDEGLNGVDLVSRILVFQRTGTEGIPMKAGRDYWWHEEAAKQRTYLPPVSCDAEDPLFLLYTSGSTGSPKGVVHTTGGYLLGAALTTRYVFDIHPEDVLFTAGDVGWITGHTYALYGPLTLGTASIIFESTPAYPDYGRYWRIIQRHKATHFYVAPTALRLIKRVGEAEIAKYDTSSLRVLGSVGEPISPDLWEWYHEKVGNKNCVICDTMWQTESGSHLIAPLAGAVPTKPGSATVPFFGINACIIDPVTGVELEGNDVEGVLAVKSPWPSMARSVWNHHDRYMDTYLKPYPGHYFTGDGAGRDHDGYYWIRGRVDDVVNVSGHRLSTSEIEASISNHENVSEAAVVGIPDELTGQTVVAYVSLKDGYLQNNATEGDAEHITPDNLRRELILQVRGEIGPFASPKTIILVRDLPRTRSGKIMRRVLRKVASNEAEQLGDLTTLANPEVVPAIISAVENQFFSQKKK

SEQ ID NO: 75 - protein, 500 aa, Saccharomyces cerevisiae
MTKLHFDTAEPVKITLPNGLTYEQPTGLFINNKFMKAQDGKTYPVEDPSTENTVCEVSSATTEDVEYAIECADRAFHDTEWATQDPRERGRLLSKLADELESQIDLVSSIEALDNGKTLALARGDVTIAINCLRDAAAYADKVNGRTINTGDGYMNFTTLEPIGVCGQIIPWNFPIMMLAWKIAPALAMGNVCILKPAAVTPLNALYFASLCKKVGIPAGVVNIVPGPGRTVGAALTNDPRIRKLAFTGSTEVGKSVAVDSSESNLKKITLELGGKSAHLVFDDANIKKTLPNLVNGIFKNAGQICSSGSRIYVQEGIYDELLAAFKAYLETEIKVGNPFDKANFQGAITNRQQFDTIMNYIDIGKKEGAKILTGGEKVGDKGYFIRPTVFYDVNEDMRIVKEEIFGPVVTVAKFKTLEEGVEMANSSEFGLGSGIETESLSTGLKVAKMLKAGTVWINTYNDFDSRVPFGGVKQSGYGREMGEEVYHAYTEVKAVRIKL

SEQ ID NO: 76 - protein, 568 aa, Zymomonas mobilis
MSYTVGTYLAERLVQIGLKHHFAVAGDYNLVLLDNLLLNKNMEQVYCCNELNCGFSAEGYARAKGAAAAVVTYSVGALSAFDAIGGAYAENLPVILISGAPNNNDHAAGHVLHHALGKTDYHYQLEMAKNITAAAEAIYTPEEAPAKIDHVIKTALREKKPVYLEIACNIASMPCAAPGPASALFNDEASDEASLNAAVEETLKFIANRDKVAVLVGSKLRAAGAEEAAVKFADALGGAVATMAAAKSFFPEENPHYIGTSWGEVSYPGVEKTMKEADAVIALAPVFNDYSTTGWTDIPDPKKLVLAEPRSVVVNGIRFPSVHLKDYLTRLAQKVSKKTGALDFFKSLNAGELKKAAPADPSAPLVNAEIARQVEALLTPNTTVIAETGDSWFNAQRMKLPNGARVEYEMQWGHIGWSVPAAFGYAVGAPERRNILMVGDGSFQLTAQEVAQMVRLKLPVIIFLINNYGYTIEVMIHDGPYNNIKNWDYAGLMEVFNGNGGYDSGAGKGLKAKTGGELAEAIKVALANTDGPTLIECFIGREDCTEELVKWGKRVAAANSRKPVNKLL

SEQ ID NO: 77 - protein, 329 aa, Streptomyces sp. CL190
MTDVRFRIIGTGAYVPERIVSNDEVGAPAGVDDDWITRKTGIRQRRWAADDQATSDLATAAGRAALKAAGITPEQLTVIAVATSTPDRPQPPTAAYVQHHLGATGTAAFDVNAVCSGTVFALSSVAGTLVYRGGYALVIGADLYSRILNPADRKTVVLFGDGAGAMVLGPTSTGTGPIVRRVALHTFGGLTDLIRVPAGGSRQPLDTDGLDAGLQYFAMDGREVRRFVTEHLPQLIKGFLHEAGVDAADISHFVPHQANGVMLDEVFGELHLPRATMHRTVETYGNTGAASIPITMDAAVRAGSFRPGELVLLAGFGGGMAASFALIEW

SEQ ID NO: 78 - protein, 2233 aa, Artificial Sequence
  Synthetic Construct
MSEESLFESSPQKMEYEITNYSERHTELPGHFIGLNTVDKLEESPLRDFVKSHGGHTVISKILIANNGIAAVKEIRSVRKWAYETFGDDRTVQFVAMATPEDLEANAEYIRMADQYIEVPGGTNNNNYANVDLIVDIAERADVDAVWAGWGHASENPLLPEKLSQSKRKVIFIGPPGNAMRSLGDKISSTIVAQSAKVPCIPWSGTGVDTVHVDEKTGLVSVDDDIYQKGCCTSPEDGLQKAKRIGFPVMIKASEGGGGKGIRQVEREEDFIALYHQAANEIPGSPIFIMKLAGRARHLEVQLLADQYGTNISLFGRDCSVQRRHQKIIEEAPVTIAKAETFHEMEKAAVRLGKLVGYVSAGTVEYLYSHDDGKFYFLELNPRLQVEHPTTEMVSGVNLPAAQLQIAMGIPMHRISDIRTLYGMNPHSASEIDFEFKTQDATKKQRRPIPKGHCTACRITSEDPNDGFKPSGGTLHELNFRSSSNVWGYFSVGNNGNIHSFSDSQFGHIFAFGENRQASRKHMVVALKELSIRGDFRTTVEYLIKLLETEDFEDNTITTGWLDDLITHKMTAEKPDPTLAVICGAATKAFLASEEARHKYIESLQKGQVLSKDLLQTMFPVDFIHEGKRYKFTVAKSGNDRYTLFINGSKCDIILRQLSDGGLLIAIGGKSHTIYWKEEVAATRLSVDSMTTLLEVENDPTQLRTPSPGKLVKFLVENGEHIIKGQPYAEIEVMKMQMPLVSQENGIVQLLKQPGSTIVAGDIMAIMTLDDPSKVKHALPFEGMLPDFGSPVIEGTKPAYKFKSLVSTLENILKGYDNQVIMNASLQQLIEVLRNPKLPYSEWKLHISALHSRLPAKLDEQMEELVARSLRRGAVFPARQLSKLIDMAVKNPEYNPDKLLGAVVEPLADIAHKYSNGLEAHEHSIFVHFLEEYYEVEKLFNGPNVREENIILKLRDENPKDLDKVALTVLSHSKVSAKNNLILAILKHYQPLCKLSSKVSAIFSTPLQHIVELESKATAKVALQAREILIQGALPSVKERTEQIEHILKSSVVKVAYGSSNPKRSEPDLNILKDLIDSNYVVFDVLLQFLTHQDPVVTAAAAQVYIRRAYRAYTIGDIRVHEGVTVPIVEWKFQLPSAAFSTFPTVKSKMGMNRAVSVSDLSYVANSQSSPLREGILMAVDHLDDVDEILSQSLEVIPRHQSSSNGPAPDRSGSSASLSNVANVCVASTEGFESEEEILVRLREILDLNKQELINASIRRITFMFGFKDGSYPKYYTFNGPNYNENETIRHIEPALAFQLELGRLSNFNIKPIFTDNRNIHVYEAVSKTSPLDKRFFTRGIIRTGHIRDDISIQEYLTSEANRLMSDILDNLEVTDTSNSDLNHIFINFIAVFDISPEDVEAAFGGFLERFGKRLLRLRVSSAEIRIIIKDPQTGAPVPLRALINNVSGYVIKTEMYTEVKNAKGEWVFKSLGKPGSMHLRPIATPYPVKEWLQPKRYKAHLMGTTYVYDFPELFRQASSSQWKNFSADVKLTDDFFISNELIEDENGELTEVEREPGANAIGMVAFKITVKTPEYPRGRQFVVVANDITFKIGSFGPQEDEFFNKVTEYARKRGIPRIYLAANSGARIGMAEEIVPLFQVAWNDAANPDKGFQYLYLTSEGMETLKKFDKENSVLTERTVINGEERFVIKTIIGSEDGLGVECLRGSGLIAGATSRAYHDIFTITLVTCRSVGIGAYLVRLGQRAIQVEGQPIILTGAPAINKMLGREVYTSNLQLGGTQIMYNNGVSHLTAVDDLAGVEKIVEWMSYVPAKRNMPVPILETKDTWDRPVDFTPTNDETYDVRWMIEGRETESGFEYGLFDKGSFFETLSGWAKGVVVGRARLGGIPLGVIGVETRTVENLIPADPANPNSAETLIQEPGQVWHPNSAFKTAQAINDFNNGEQLPMMILANWRGFSGGQRDMFNEVLKYGSFIVDALVDYKQPIIIYIPPTGELRGGSWVVVDPTINADQMEMYADVNARAGVLEPQGMVGIKFRREKLLDTMNRLDDKYRELRSQLSNKSLAPEVHQQISKQLADRERELLPIYGQISLQFADLHDRSSRMVAKGVISKELEWTEARRFFFWRLRRRLNEEYLIKRLSHQVGEASRLEKIARIRSWYPASVDHEDDRQVATWIEENYKTLDDKLKGLKLESFAQDLAKKIRSDHDNAIDGLSEVIKMLSTDDKEKLLKTLK

SEQ ID NO: 79 - DNA, 500 bp, Artificial Sequence
  Synthetic Construct
tggaactttcagtaatacgcttaactgctcattgctatattgaagtacggattagaagccgccgagcgggcgacagccctccgacggaagactctcctccgtgcgtcctggtcttcaccggtcgcgttcctgaaacgcagatgtgcctcgcgccgcactgctccgaacaataaagattctacaatactagcttttatggttatgaagaggaaaaattggcagtaacctggccccacaaaccttcaaatcaacgaatcaaattaacaaccataggataataatgcgattagttttttagccttatttctggggtaattaatcagcgaagcgatgatttttgatctattaacagatatataaatgcaaaagctgcataaccactttaactaatactttcaacattttcggtttgtattacttcttattcaaatgtcataaaagtatcaacaaaaaattgttaatatacctctatactttaacgtcaaggagaaaaaactata

SEQ ID NO: 80 - DNA, 501 bp, Artificial Sequence
  Synthetic Construct
catcgcttcgctgattaattaccccagaaataaggctaaaaaactaatcgcattattatcctatggttgttaatttgattcgttgatttgaaggtttgtggggccaggttactgccaatttttcctcttcataaccataaaagctagtattgtagaatctttattgttcggagcagtgcggcgcgaggcacatctgcgtttcaggaacgcgaccggtgaagaccaggacgcacggaggagagtcttccgtcggagggctgtcgcccgctcggcggcttctaatccgtacttcaatatagcaatgagcagttaagcgtattactgaaagttccaaagagaaggtttttttaggctaagataatggggctctttacatttccacaacatataagtaagattagatatggatatgtatatggtggtattgccatgtaatatgattattaaacttctttgcgtccatccaaaaaaaaagtaagaatttttgaaaattcaatataa

SEQ ID NO: 81 - DNA, 509 bp, Artificial Sequence
  Synthetic Construct
ggcttaagtaggttgcaatttctttttctattagtagctaaaaatgggtcacgtgatctatattcgaaaggggcggttgcctcaggaaggcaccggcggtctttcgtccgtgcggagatatctgcgccgttcaggggtccatgtgccttggacgatattaaggcagaaggcagtatcggggcggatcactccgaaccgagattagttaagcccttcccatctcaagatggggagcaaatggcattatactcctgctagaaagttaactgtgcacatattcttaaattatacaatgttctggagagctattgtttaaaaaacaaacatttcgcaggctaaaatgtggagataggattagttttgtagacatatataaacaatcagtaattggattgaaaatttggtgttgtgaattgctcttcattatgcaccttattcaattatcatcaagaatagcaatagttaagtaaacacaagattaacataataaaaaaaataattctttcata

SEQ ID NO: 82 - DNA, 498 bp, Artificial Sequence
  Synthetic Construct
ttttactattatcttctacgctgacagtaatatcaaacagtgacacatattaaacacagtggtttctttgcataaacaccatcagcctcaagtcgtcaagtaaagatttcgtgttcatgcagatagataacaatctatatgttgataattagcgttgcctcatcaatgcgagatccgtttaaccggaccctagtgcacttaccccacgttcggtccactgtgtgccgaacatgctccttcactattttaacatgtggaattcttgaaagaatgaaatcgccatgccaagccatcacacggtcttttatgcaattgattgaccgcctgcaacacataggcagtaaaatttttactgaaacgtatataatcatcataagcgacaagtgaggcaacacctttgttaccacattgacaaccccaggtattcatacttcctattagcggaatcaggagtgcaaaaagagaaaataaaagtaaaaaggtagggcaacacatagt

SEQ ID NO: 83 - DNA, 501 bp, Artificial Sequence
  Synthetic Construct
ggacggtagcaacaagaatatagcacgagccgcgaagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctcattagatatattttctgtcattttccttaacccaaaaataagggaaagggtccaaaaagcgctcggacaactgttgaccgtgatccgaaggactggctatacagtgttcacaaaatagccaagctgaaaataatgtgtagctatgttcagttagtttggctagcaaagatataaaagcaggtcggaaatatttatgggcattattatgcagagcatcaacatgataaaaaaaaacagttgaatattccctcaaaa

SEQ ID NO: 84 - DNA, 489 bp, Artificial Sequence
  Synthetic Construct
gcgacacagagatgacagacggtggcgcaggatccggtttaaacgaggatcccttaagtttaaacaacaacagcaagcaggtgtgcaagacactagagactcctaacatgatgtatgccaataaaacacaagagataaacaacattgcatggaggccccagaggggcgattggtttgggtgcgtgagcggcaagaagtttcaaaacgtccgcgtcctttgagacagcattcgcccagtattttttttattctacaaaccttctataatttcaaagtatttacataattctgtatcagtttaatcaccataatatcgttttctttgtttagtgcaattaatttttcctattgttacttcgggcctttttctgttttatgagctattttttccgtcatccttccccagattttcagcttcatctccagattgtgtctacgtaatgcacgccatcattttaagagaggacagagaagcaagcctcctgaaag

SEQ ID NO: 85 - DNA, 1253 bp, Artificial Sequence
  Synthetic Construct
gatgatggacactagtgtgtcgagaatgtatcaactatatatagtcctaatgccacacaaatatgaagtgggggaagcccattcttaatccggctcaattttggtgcgtgatcgcggcctatgtttgcttccagaaaaagcttagaataatatttctcacctttgatggaatgctcgcgagtgctcgttttgattaccccatatgcattgttgcagcatgcaagcactattgcaagccacgcatggaagaaatttgcaaacacctatagccccgcgttgttgaggaggtggacttggtgtaggaccataaagctgtgcactactatggtgagctctgtcgtctggtgaccttctatctcaggcacatcctcgtttttgtgcatgaggttcgagtcacgcccacggcctattaatccgcgaaataaatgcgaaatctaaattatgacgcaaggctgagagattctgacacgccgcatttgcggggcagtaattatcgggcagttttccggggttcgggatggggtttggagagaaagttcaacacagaccaaaacagcttgggaccacttggatggaggtccccgcagaagagctctggcgcgttggacaaacattgacaatccacggcaaaattgtctacagttccgtgtatgcggatagggatatcttcgggagtatcgcaataggatacaggcactgtgcagattacgcgacatgatagctttgtatgttctacagactctgccgtagcagtctagatataatatcggagttttgtagcgtcgtaaggaaaacttgggttacacaggtttcttgagagccctttgacgttgattgctctggcttccatccaggccctcatgtggttcaggtgcctccgcagtggctggcaagcgtgggggtcaattacgtcacttctattcatgtaccccagactcaattgttgacagcaatttcagcgagaattaaattccacaatcaattctcgctgaaataattaggccgtgatttaattctcgctgaaacagaatcctgtctggggtacagataacaatcaagtaactattatggacgtgcataggaggtggagtccatgacgcaaagggaaatattcattttatcctcgcgaagttgggatgtgtcaaagcgtcgcgctcgctatagtgatgagaatgtctttagtaagcttaagccatataaagaccttccgcctccatatttttttttatccctcttgacaatattaattcctt

SEQ ID NO: 86 - DNA, 1253 bp, Artificial Sequence
  Synthetic Construct
aaggaattaatattgtcaagagggataaaaaaaaatatggaggcggaaggtctttatatggcttaagcttactaaagacattctcatcactatagcgagcgcgacgctttgacacatcccaacttcgcgaggataaaatgaatatttccctttgcgtcatggactccacctcctatgcacgtccataatagttacttgattgttatctgtaccccagacaggattctgtttcagcgagaattaaatcacggcctaattatttcagcgagaattgattgtggaatttaattctcgctgaaattgctgtcaacaattgagtctggggtacatgaatagaagtgacgtaattgacccccacgcttgccagccactgcggaggcacctgaaccacatgagggcctggatggaagccagagcaatcaacgtcaaagggctctcaagaaacctgtgtaacccaagttttccttacgacgctacaaaactccgatattatatctagactgctacggcagagtctgtagaacatacaaagctatcatgtcgcgtaatctgcacagtgcctgtatcctattgcgatactcccgaagatatccctatccgcatacacggaactgtagacaattttgccgtggattgtcaatgtttgtccaacgcgccagagctcttctgcggggacctccatccaagtggtcccaagctgttttggtctgtgttgaactttctctccaaaccccatcccgaaccccggaaaactgcccgataattactgccccgcaaatgcggcgtgtcagaatctctcagccttgcgtcataatttagatttcgcatttatttcgcggattaataggccgtgggcgtgactcgaacctcatgcacaaaaacgaggatgtgcctgagatagaaggtcaccagacgacagagctcaccatagtagtgcacagctttatggtcctacaccaagtccacctcctcaacaacgcggggctataggtgtttgcaaatttcttccatgcgtggcttgcaatagtgcttgcatgctgcaacaatgcatatggggtaatcaaaacgagcactcgcgagcattccatcaaaggtgagaaatattattctaagctttttctggaagcaaacataggccgcgatcacgcaccaaaattgagccggattaagaatgggcttcccccacttcatatttgtgtggcattaggactatatatagttgatacattctcgacacactagtgtccatcatc

SEQ ID NO: 87 - DNA, 665 bp, Artificial Sequence
  Synthetic Construct
gcgcctcaagaaaatgatgctgcaagaagaattgaggaaggaactattcatcttacgttgtttgtatcatcccacgatccaaatcatgttacctacgttaggtacgctaggaactaaaaaaagaaaagaaaagtatgcgttatcactcttcgagccaattcttaattgtgtggggtccgcgaaaatttccggataaatcctgtaaactttaacttaaaccccgtgtttagcgaaattttcaacgaagcgcgcaataaggagaaatattatctaaaagcgagagtttaagcgagttgcaagaatctctacggtacagatgcaacttactatagccaaggtctattcgtattactatggcagcgaaaggagctttaaggttttaattaccccatagccatagattctactcggtctatctatcatgtaacactccgttgatgcgtactagaaaatgacaacgtaccgggcttgagggacatacagagacaattacagtaatcaagagtgtacccaactttaacgaactcagtaaaaaataaggaatgtcgacatcttaattttttatataaagcggtttggtattgattgtttgaagaattttcgggttggtgtttctttctgatgctacatagaagaacatcaaacaactaaaaaaatagtataat

SEQ ID NO: 88 - DNA, 785 bp, Artificial Sequence
  Synthetic Construct
attatactatttttttagttgtttgatgttcttctatgtagcatcagaaagaaacaccaacccgaaaattcttcaaacaatcaataccaaaccgctttatataaaaaattaagatgtcgacattccttattttttactgagttcgttaaagttgggtacactcttgattactgtaattgtctctgtatgtccctcaagcccggtacgttgtcattttctagtacgcatcaacggagtgttacatgatagatagaccgagtagaatctatggctatggggtaattaaaaccttaaagctcctttcgctgccatagtaatacgaatagaccttggctatagtaagttgcatctgtaccgtagagattcttgcaactcgcttaaactctcgcttttagataatatttctccttattgcgcgcttcgttgaaaatttcgctaaacacggggtttaagttaaagtttacaggatttatccggaaattttcgcggaccccacacaattaagaattggctcgaagagtgataacgcatacttttcttttctttttttagttcctagcgtacctaacgtaggtaacatgatttggatcgtgggatgatacaaacaacgtaagatgaatagttccttcctcaattcttcttgcagcatcattttcttgaggcgctctgggcaaggtataaaaagttccattaatacgtctctaaaaaattaaatcatccatctcttaagcagtttttttgataatctcaaatgtacatcagtcaagcgtaactaaattacataa

SEQ ID NO: 89 - DNA, 875 bp, Artificial Sequence
  Synthetic Construct
ttatgtattttagttacgcttgactgatgtacatttgagattatcaaaaaaactgcttaagagatagatggtttaattttttagagacgtattaatggaactttttataccttgcccagagcgcctcaagaaaatgatgctgaaagaagaattgaggaaggaactactcatcttacgttgtttgtatcatcccacgatccaaatcatgttacctacgttaggtacgctaggaactgaaaaaagaaaagaaaagtatgcgttatcactcttcgagccaattcttaattgtgtggggtccgcgaaaacttccggataaatcctgtaaacttaaacttaaaccccgtgtttagcgaaattttcaacgaagcgcgcaataaggagaaatattatataaaagcgagagtttaagcgaggttgcaagaatctctacggtacagatgcaacttactatagccaaggtctattcgtattggtatccaagcagtgaagctactcaggggaaaacatattttcagagatcaaagttatgtcagtctctttttcatgtgtaacttaacgtttgtgcaggtatcataccggcctccacataatttttgtggggaagacgttgttgtagcagtctccttatactctccaacaggtgtttaaagacttcttcaggcctcatagtctacatctggagacaacattagatagaagtttccacagaggcagctttcaatatactttcggctgtgtacatttcatcctgagtgagcgcatattgcataagtactcagtatataaagagacacaatatactccatacttgttgtgagtggttttagcgtattcagtataacaataagaattacatccaagactattaattaact

SEQ ID NO: 90 - DNA, 875 bp, Artificial Sequence
  Synthetic Construct
agttaattaatagtcttggatgtaattcttattgttatactgaatacgctaaaaccactcacaacaagtatggagtatattgtgtctctttatatactgagtacttatgcaatatgcgctcactcaggatgaaatgtacacagccgaaagtatattgaaagctgcctctgtggaaacttctatctaatgttgtctccagatgtagactatgaggcctgaagaagtctttaaacacctgttggagagtataaggagactgctacaacaacgtcttccccacaaaaattatgtggaggccggtatgatacctgcacaaacgttaagttacacatgaaaaagagactgacataactttgatctctgaaaatatgttttcccctgagtagcttcactgcttggataccaatacgaatagaccttggctatagtaagttgcatctgtaccgtagagattcttgcaacctcgcttaaactctcgcttttatataatatttctccttattgcgcgcttcgttgaaaatttcgctaaacacggggtttaagtttaagtttacaggatttatccggaagttttcgcggaccccacacaattaagaattggctcgaagagtgataacgcatacttttcttttcttttttcagttcctagcgtacctaacgtaggtaacatgatttggatcgtgggatgatacaaacaacgtaagatgagtagttccttcctcaattcttctttcagcatcattttcttgaggcgctctgggcaaggtataaaaagttccattaatacgtctctaaaaaattaaaccatctatctcttaagcagtttttttgataatctcaaatgtacatcagtcaagcgtaactaaaatacataa